(19) United States
(12) Patent Application Publication
(10) Pub. No.: US 2026/0021202 A1
(43) Pub. Date: Jan. 22, 2026

(54) COMPOSITIONS AND METHODS FOR TREATING NON-AGE-ASSOCIATED HEARING IMPAIRMENT IN A HUMAN SUBJECT

(71) Applicant: Akouos, Inc., Boston, MA (US)

(72) Inventors: Robert NG, Boston, MA (US); Jennifer WELLMAN, Boston, MA (US); Emmanuel John SIMONS, Boston, MA (US); William SEWELL, Boston, MA (US); Michelle VALERO, Boston, MA (US); Ann HICKOX, Boston, MA (US)

(21) Appl. No.: 18/866,969

(22) PCT Filed: May 19, 2023
(86) PCT No.: PCT/US2023/067220

§ 371 Date: Nov. 18, 2024

Related U.S. Application Data
(60) Provisional application No. 63/343,991, filed on May 19, 2022.

(51) Int. Cl. A61K 48/00; A61K 9/00; A61K 38/17; A61K 47/02; A61K 47/10; A61P 27/16; C07K 14/005; C12N 15/86

(52) U.S. Cl. CPC A61K 48/005; A61K 9/0046; A61K 38/1709; A61K 47/02; A61K 47/10; A61K 48/0075; A61K 48/0083; A61P 27/16; C07K 14/005; C12N 15/86; C12N 2750/14122; C12N 2750/14143; C12N 2800/40; C12N 2830/50

(57) ABSTRACT

Provided herein are compositions that comprise a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs, wherein the composition comprises about 4.1E10-4.1E12 total vg or about 8.1E10-8.1E12 total vg, and the use of these compositions to treat hearing loss in a subject.

### CROSS-REFERENCE TO RELATED APPLICATIONS

This application claims the benefit of U.S. Provisional Application No. 63/343,991, filed May 19, 2022, which is hereby incorporated by reference in its entirety.

### SEQUENCE LISTING

The content of the electronically submitted sequence listing in XML format (Name: 4833.017PC02_Seqlisting_ST26.xml; Size: 816,150 bytes; and Date of Creation: May 17, 2023) filed with the application is incorporated herein by reference in its entirety.

### TECHNICAL FIELD

The present disclosure relates generally to the use of nucleic acids to treat hearing loss in a human subject.

### BACKGROUND OF THE DISCLOSURE

The ear is a complex organ, classically described as including the outer ear, the middle ear, the inner ear, the hearing (acoustic) nerve and the auditory system (which processes sound as it travels from the ear to the brain). In addition to detecting sound, the ear also helps to maintain balance. Thus, disorders of the inner ear can cause hearing loss, tinnitus, vertigo and imbalance.

Hearing loss is one of the most common human sensory deficits, and can occur for many reasons. Some people may be born with hearing loss while others may lose their hearing slowly over time. Presbycusis (also spelled presbyacusis) is age-related hearing loss. Approximately 36 million American adults report some degree of hearing loss, and one in three people older than 60 and half of those older than 85 experience hearing loss.

Hearing loss can be the result of environmental factors or a combination of genetic and environmental factors. About half of all people who have tinnitus—phantom noises in their auditory system (ringing, buzzing, chirping, humming, or beating)—also have an over-sensitivity to/reduced tolerance for certain sound frequency and volume ranges, known as hyperacusis (also spelled hyperacousis). Environmental causes of hearing loss include certain medications, specific infections before or after birth, and exposure to loud noise over an extended period. Hearing loss can result from noise, ototoxic agents, presbyacusis, disease, infection or cancers that affect specific parts of the ear.

Approximately 1.5 in 1,000 children are born with profound hearing loss, and another two to three per 1,000 children are born with partial hearing loss (Smith et al., 2005, Lancet 365:879-890). More than half of these cases are attributed to a genetic basis (Di Domenico, et al., 2011, J. Cell. Physiol. 226:2494-2499).

Nonsyndromic deafness is hearing loss that is not associated with other signs and symptoms. In contrast, syndromic deafness involves hearing loss that occurs with abnormalities in other parts of the body. Most cases of genetic deafness (70 percent to 80 percent) are nonsyndromic; the remaining cases are caused by specific genetic syndromes.

Hearing loss can be conductive (arising from the ear canal or middle ear), sensorineural (arising from the inner ear or auditory nerve), or mixed. Most forms of nonsyndromic deafness are associated with permanent hearing loss caused by damage to structures in the inner ear (sensorineural deafness). Some human sensorineural hearing loss is caused by abnormalities in the hair cells of the organ of Corti in the cochlea. There are also sensorineural hearing impairments that involve the eighth cranial nerve (the vestibulocochlear nerve) or the auditory portions of the brain. Most sensorineural hearing loss is due to poor hair cell function. The hair cells may be abnormal at birth, or damaged during the lifetime of an individual. There are both external causes of damage, like noise trauma and infection, and intrinsic abnormalities, like congenital mutations to genes that play an important role in cochlear anatomy or physiology.

Hearing loss that results from changes in the middle ear is called conductive hearing loss. Some forms of nonsyndromic deafness involve changes in both the inner ear and the middle ear, called mixed hearing loss. Hearing loss that is present before a child learns to speak is classified as prelingual or congenital. Hearing loss that occurs after the development of speech is classified as postlingual. Most autosomal recessive loci cause prelingual severe-to-profound hearing loss.

Nonsyndromic deafness can have different patterns of inheritance, and can occur at any age. Types of nonsyndromic deafness are named according to their inheritance patterns. Autosomal dominant forms are designated DFNA, autosomal recessive forms are DFNB, and X-linked forms are DFN. Each type is also numbered in the order in which it was described. For example, DFNA1 was the first described autosomal dominant type of nonsyndromic deafness.

Auditory neuropathy spectrum disorder (ANSD), a hearing disorder characterized by normal outer hair cells function and abnormal or absent auditory brain stem response, is one of the most common diseases leading to hearing and speech communication barriers in infants and young children. Approximately 10 percent of children with permanent hearing loss may have ANSD. The OTOF gene is the first gene identified for autosomal recessive non-syndromic ANSD, and mutations in OTOF have been found to account for approximately 5% of all cases of autosomal recessive nonsydromic hearing loss in some populations (Rodriguez-Ballesteros et al. 2008 Human Mut 29 (6): 823-831).

The causes of nonsyndromic deafness are complex. Researchers have identified more than 30 genes that, when altered, are associated with nonsyndromic deafness; however, some of these genes have not been fully characterized. Different mutations in the same gene can be associated with different types of hearing loss, and some genes are associated with both syndromic and nonsyndromic deafness.

For example, genes associated with nonsyndromic deafness include, but are not limited to, ATP2B2, ACTG1, CDH23, CLDN14, COCH, COL11A2, DFNA5, DFNB31, DFNB59, ESPN, EYA4, GJB3, KCNQ4, LHFPL5, MYO1A, MYO15A, MYO6,

MYO7A, OTOF, PCDH15, SLC26A4, STRC, TECTA, TMC1, TMIE, TMPRSS3, TRIOBP, USH1C, and WFS1.

OTOF-related deafness (DFNB9 nonsyndromic hearing loss) is characterized by two phenotypes: prelingual nonsyndromic hearing loss and, less frequently, temperature-sensitive nonsyndromic auditory neuropathy (TS-NSAN). Another form of progressive hearing impairment is associated with a mutation in the otoferlin gene (e.g., a I1573T mutation or a P1987R mutation, and/or a E1700Q mutation), or is not temperature sensitive.

Treatments for hearing loss currently consist of hearing amplification for mild to severe losses and cochlear implantation for severe to profound losses (Kral and O'Donoghue, 2010, N. Engl. J. Med. 363:1438-1450). To date, a majority of the research in this arena has focused on cochlear hair cell regeneration, applicable to the most common forms of hearing loss, including presbycusis, noise damage, infection, and ototoxicity.

A long-felt need remains for agents and methods for preventing or reversing deafness.

### BRIEF DESCRIPTION OF THE DRAWING

FIG. 1 is a schematic of the AAVAnc80-hOTOF construct comprising two recombinant vectors (AAVAnc80-5′hOTOF and AAVAnc80-3′hOTOF). The upstream DNA genome (AAVAnc80-5′hOTOF) includes a eukaryotic expression cassette encoding the following promoter and regulatory sequences: the cytomegalovirus (CMV) early enhancer element; the chicken beta actin (CBA) gene sequence located between 5′ flanking region and the proximal region of the second exon; and 3′ splice sequence derived from the rabbit beta globin (RBG) gene; the human OTOF (hOTOF) coding sequence, exons 1 through 21 (inclusive); a synthetic splice donor (SD); and a 77-base pair (bp) AK recombinogenic sequence. The downstream DNA genome (AAVAnc80-3′hOTOF) includes a eukaryotic expression cassette encoding the following: the same 77-bp AK recombinogenic sequence; a synthetic splice acceptor (SA); the human OTOF (hOTOF) coding sequence, exons 22 through 45 (inclusive) and exon 47, excluding non-coding exon 46; and the bovine growth hormone (bGH) polyadenylation (pA) signal. Each expression cassette is flanked by AAV2 inverted terminal repeats (ITRs). Abbreviations: AK=recombinogenic sequence; bGH=bovine growth hormone; CAG=the cytomegalovirus (CMV) early enhancer element, the promoter and the first exon of the chicken beta actin (CBA) gene, and a chimeric intron composed of the splice donor (SD) and the first intron of CBA and the splice acceptor (SA) sequence derived from the rabbit beta globin gene which together are commonly referred to as the CAG promoter; ITR=inverted terminal repeat; OTOF=otoferlin gene; pA=polyadenylation signal; SA=splice acceptor; SD=splice donor.

FIG. 2 illustrates a perspective of a device for delivering fluid to an inner ear, according to aspects of the present disclosure.

FIG. 3 illustrates a sideview of a bent needle sub-assembly, according to aspects of the present disclosure.

FIG. 4 illustrates a perspective view of a device for delivering fluid to an inner ear, according to aspects of the present disclosure.

FIG. 5 illustrates a perspective view of a bent needle sub-assembly coupled to the distal end of a device, according to aspects of the present disclosure.

FIGS. 6A-6C are immunohistochemical images of outer hair cells (OHCs) and inner hair cells (IHCs) in wildtype mice administered vehicle (FIG. 6A) or Otof^{-/-} mice administered vehicle (FIG. 6B) or AAVAnc80.AKhOTOF5 and AAVAnc80.AKhOTOF3 (FIG. 6C). Myo7a (green) is a marker for inner hair cells. Phalloidin (blue) is a marker for inner and outer hair cells. Otoferlin expression is shown in green. Co-localization of otoferlin with Myo7a in inner hair cells is shown in orange/yellow.

FIGS. 7A-7B are immunohistochemical images of outer hair cells (OHCs) and inner hair cells (IHCs) in wildtype non-human primates administered either vehicle (FIG. 7A) or AAVAnc80.AKhOTOF5 and AAVAnc80.AKhOTOF3-Flag (FIG. 7B). Flag is shown in yellow. Wildtype otoferlin expression is shown in pink. Phalloidin (blue) is a marker for inner and outer hair cells.

FIGS. 8A-8B are graphs showing auditory brainstem response (ABRs) in Otof^{-/-} mice 15, 30, 45, and 60 days (FIG. 8A) or 1, 2, 3, 4.5, or 6 months (FIG. 8B) post-administration of either vehicle or AAVAnc80.AKhOTOF5 and AAVAnc80.AKhOTOF3.

FIGS. 9A-9E are graphs showing the biodistribution of Otoferlin-Flag in non-human primates adminstered either vehicle, AAVAnc80.AKhOTOF5 and AAVAnc80.AKhOTOF3-Flag, or a spike-in control. Otoferlin-Flag mRNA levels in the mandibular lymph node (FIG. 9A), liver (FIG. 9B), and spleen (FIG. 9C) were measured by RT-qPCR. Otoferlin-Flag protein levels in the liver (FIG. 9D) and spleen (FIG. 9E) were measured by western blotting.

FIGS. 10A-10F are graphs showing the percent survival of inner and outer hair cells in non-human primates (FIGS. 10A-10B) or Otof−/− mice (FIGS. 10D-10E) adminstered either vehicle or AAVAnc80.AKhOTOF5 and AAVAnc80.AKhOTOF3. The cochlear regions evaluated are shown in FIGS. 10C and 10F.

FIGS. 11A-11B are graphs showing the auditory brainstem response (ABR) (FIG. 11A) or distortion product otoacoustic emission (DPOAE) (FIG. 11B) of non-human primates pre- and 6 months post-intracochlear administration of either vehicle or the AAVAnc80.AKhOTOF5 and AAVAnc80.AKhOTOF3-Flag.

### SUMMARY

Certain aspects of the disclosure are directed to a composition comprising: a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition comprises about 4.1E10-4.1E12 total vg or about 8.1E10-8.1E12 total vg.

In some aspects, the composition comprises about 4.1E10-4.1E12 total vg/cochlea total vg/cochlea. In some aspects the composition comprises about 4.1E11 total vg/cochlea. In other aspects, the composition comprises about 8.1E10-8.1E12 total vg/cochlea. In some aspects, the composition comprises about 8.1E11 total vg/cochlea.

In some aspects, the concentration of the composition comprises about 4.5E11-4.5E13 total vg/mL or about 9E11-9E13 total vg/mL. In some aspects, the concentration of the composition comprises about 4.5E11-4.5E13 total vg/mL. In some aspects, the concentration of the composition comprises about 4.5E12 total vg/mL In other aspects, the concentration of the composition comprises about 9E11-9E13 total vg/mL. In some aspects, the composition comprises about 9E12 total vg/mL In some aspects, the composition comprises the first rAAV vector genome and the second rAAV vector genome in about a 1:1 ratio.

In some aspects, the composition comprises about 4.1E11 total vg. In some aspects, the composition comprises about 8.1E11 total vg.

Certain aspects of the disclosure are directed to a composition comprising: a) a first rAAV vector genome comprising a first expression cassette comprising a promoter, a first coding sequence that encodes an N-terminal portion of an otoferlin protein positioned 3′ of the promoter, and a splicing donor signal sequence positioned at the 3′ end of the first coding sequence; and b) a second rAAV vector genome comprising a second expression cassette comprising a splicing acceptor signal sequence, a second coding sequence that encodes a C-terminal portion of an otoferlin protein positioned at the 3′ end of the splicing acceptor signal sequence, and a polyadenylation sequence at the 3′ end of the second coding sequence, wherein the composition is formulated for intra-cochlear administration.

In some aspects, the composition comprises one or more pharmaceutically acceptable carriers, diluents, or excipients. In some aspects, the composition is formulated to comprise a synthetic perilymph solution.

In some aspects, the composition comprises one or more buffering agents and one or more surfactants. In some aspects, the buffering agents are selected from monopotassium phosphate, sodium phosphate dibasic, potassium chloride, sodium chloride, Tris HCl, Tris base, histidine, boric acid, citric acid, glycine, HEPES, and MOPS. In some aspects, the surfactant is selected from poloxamer 188, labrasol, tween, ethanol, pluronic F68, and polyethyelene glycol. In some aspects, the composition comprises monopotassium phosphate, sodium phosphate dibasic, potassium chloride, sodium chloride, and Poloxamer 188.

In some aspects, the formulation comprises: a) about 1.35-1.65 mM monopotassium phosphate; b) about 7.29-8.91 mM sodium phosphate dibasic; c) about 2.43-2.97 mM potassium chloride; d) about 154.8-189.2 mM sodium chloride; and e) about 0.0001%-0.01% Poloxamer 188. In some aspects, the formulation comprises: a) about 1.5 mM monopotassium phosphate; b) about 8.1 mM sodium phosphate dibasic; c) about 2.7 mM potassium chloride; d) about 172 mM sodium chloride; and e) about 0.001% Poloxamer 188.

In some aspects, the composition comprises about 4.1E10-8.1E12 total vg/cochlea. In some aspects, the composition comprises about 4.1E10-4.1E12 total vg/cochlea or about 8.1E10-8.1E12 total vg/cochlea. In some aspects, the composition comprises about 4.1E11 total vg/cochlea. In other aspects, the composition comprises about 8.1E11 total vg/cochlea.

In some aspects, the concentration of the composition comprises about 4.5E11-4.5E13 total vg/mL. In some aspects, the concentration of the composition comprises about 4.5E12 total vg/mL. In other aspects, the concentration of the composition comprises about 9E11-9E13 total vg/mL. In some aspects, the concentration of the composition comprises about 9E12 total vg/mL.

In some aspects, the composition comprises the first rAAV vector genome and the second rAAV vector genome in about a 1:1 ratio.

In some aspects, the nucleic acid sequence comprising 5′ portion of the otoferlin gene has at least 80%, at least 85%, at least 90%, at least 91%, at least 92%, at least 93%, at least 94%, at least 95%, at least 96%, at least 97%, at least 98%, at least 99%, or 100% identity to SEQ ID NO: 101. In some aspects, the nucleic acid sequence comprising 3′ portion of the otoferlin gene has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 107.

In some aspects, the promoter is selected from a constitutive promoter, an inducible promoter, or a tissue-specific promoter. In some aspects, the promoter is a constitutive promoter. In some aspects, the constitutive promoter is selected from a CAG, CBA, or CMV promoter. In some aspects, the constitutive promoter is a CAG promoter.

In some aspects, the first rAAV vector comprises a splicing donor site and a recombinogenic sequence. In some aspects, the second rAAV vector comprises a splicing acceptor site, a recombinogenic sequence, and a polyadenylation sequence.

In some aspects, the splicing donor site has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 102. In some aspects, the splicing acceptor site has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 106.

In some aspects, the recombinogenic sequence has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 103.

In some aspects, the polyadenylation sequence is selected from a bovine growth hormone, human growth hormone, mouse-β-globin, mouse-α-globin, polyomavirus, SV40, or synthetic polyadenylation sequence. In some aspects, the polyadenylation is a bovine growth hormone polyadenylation sequence. In some aspects, the polyadenylation sequence has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 108.

In some aspects, the ITRs are selected from any one of AAV1, AAV2, AAV3, AAV4, AAV5, AAV6, AAV7, AAV8, AAV9, AAVrh8, AAVrh10, AAVrh39, AAVrh43, or Anc80 ITRs. In some aspects, the ITRs are AAV2 ITRs.

In some aspects, the first expression cassette has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 96.

In some aspects, the second expression cassette has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 105.

In some aspects, the first and second rAAV vectors are each encapsulated by an AAV capsid. In some aspects, the AAV capsid encapsulating the first rAAV vector is a serotype selected from any one of AAV1, AAV2, AAV3, AAV4, AAV5, AAV6, AAV7, AAV8, AAV9, AAVrh8, AAVrh10, AAVrh39, AAVrh43, or Anc80. In some aspects, the AAV capsid encapsulating the second rAAV vector is a serotype selected from any one of AAV1, AAV2, AAV3, AAV4, AAV5, AAV6, AAV7, AAV8, AAV9, AAVrh8, AAVrh10, AAVrh39, AAVrh43, or Anc80. In some aspects, the first rAAV vector is encapsulated by an Anc80 capsid and the second rAAV vector is encapsulated by an Anc80 capsid. In some aspects, the Anc80 capsids comprise the polypeptide sequence of SEQ ID NO: 109.

In some aspects, the composition is formulated for intra-cochlear administration. In some aspects, the composition comprises one or more pharmaceutically acceptable carriers, diluents, or excipients. In some aspects, the composition is formulated to comprise a synthetic perilymph solution.

In some aspects, the composition comprises one or more buffering agents and one or more surfactants. In some aspects, the buffering agents are selected from monopotassium phosphate, sodium phosphate dibasic, potassium chloride, sodium chloride, Tris HCl, Tris base, histidine, boric acid, citric acid, glycine, HEPES, and MOPS. In some aspects, the surfactant is selected from poloxamer 188, labrasol, tween, ethanol, pluronic F68, and polyethyelene glycol. In some aspects, the composition comprises monopotassium phosphate, sodium phosphate dibasic, potassium chloride, sodium chloride, and Poloxamer 188.

In some aspects, the formulation comprises: a) about 1.35-1.65 mM monopotassium phosphate; b) about 7.29-8.91 mM sodium phosphate dibasic; c) about 2.43-2.97 mM potassium chloride; d) about 154.8-189.2 mM sodium chloride; and e) about 0.0001%-0.01% Poloxamer 188. In some aspects, the formulation comprises: a) about 1.5 mM monopotassium phosphate; b) about 8.1 mM sodium phosphate dibasic; c) about 2.7 mM potassium chloride; d) about 172 mM sodium chloride; and e) about 0.001% Poloxamer 188.

Certain aspects of the disclosure are directed to a method of treating hearing loss in a subject having a defective otoferlin gene, comprising administering a composition comprising: a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition comprises about 4.1E10-4.1E12 total vg or about 8.1E10-8.1E12 total vg, into the cochlea of the subject, wherein the first and second rAAV vectors are capable of constituting a polypeptide messenger RNA encoding a full length human otoferlin protein in the subject.

In some aspects, the method further comprises, prior to the administering step, determining that the subject has a defective otoferlin gene. In some aspects, the defective otoferlin gene comprises a mutation that results in a decrease in the expression and/or activity of an otoferlin protein encoded by the gene.

In some aspects, the method serves to mitigate or prevent secondary degeneration of one or more cochlear structures.

Certain aspects of the disclosure are directed to a method of expressing a recombinant full-length otoferlin protein in a mammalian cell, comprising administering a composition comprising: a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition comprises about 4.1E10-4.1E12 total vg or about 8.1E10-8.1E12 total vg, to the mammalian cell, wherein the first and second rAAV vectors are capable of constituting a polypeptide messenger RNA encoding a full length human otoferlin protein in the mammalian cell.

In some aspects, the mammalian cell is a cochlear cell. In some aspects, the mammalian cell is an inner ear hair cell. In some aspects, the subject is a mammal. In some aspects, the subject is a human.

In some aspects, the composition is administered in a single dose. In some aspects, the composition is administered in a plurality of doses. In some aspects, the composition is administered in 2, 3, 4, 5, 6, 7, 8, 9, or 10 doses.

In some aspects, a single dose comprises about 0.01 mL-0.2 mL. In some aspects, a single dose comprises about 0.09 mL.

In some aspects, the composition is administered as an injection to the round window membrane. In some aspects, the composition is administered in a single injection. In some aspects, the composition is administered in a plurality of injections. In some aspects, the composition is administered in 2, 3, 4, 5, 6, 7, 8, 9, or 10 injections.

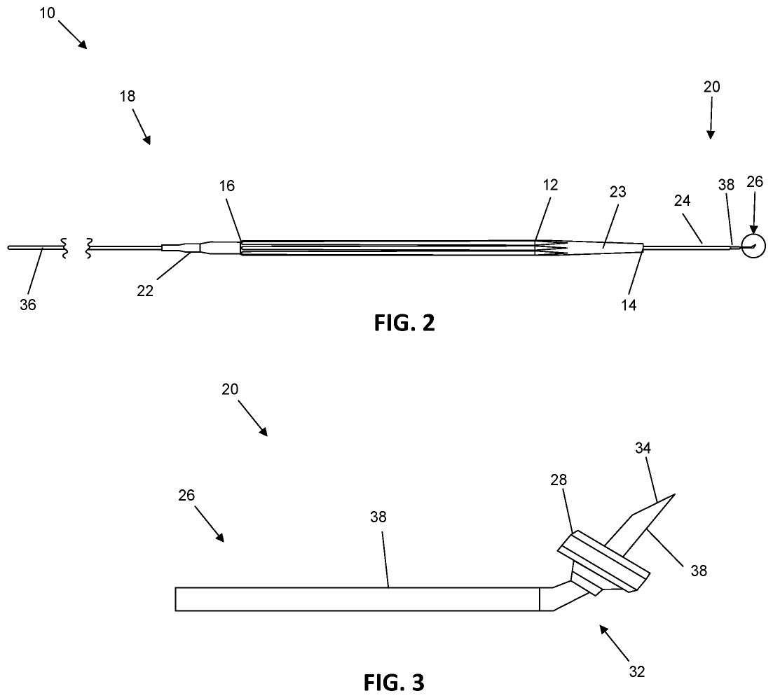

In some aspects, the composition is administered through the use of a medical device. In some aspects, the device is a device as shown in FIGS. 2-5. In some aspects, the device is a microcatheter.

In some aspects, the composition is delivered at a controlled flow rate.

In some aspects, the subject is between 2 and 17 years of age.

In some aspects, the administration of the composition improves the subject's auditory brainstem response (ABR) threshold response, age-appropriate behavioral audiometry, tympanometry, and/or word/sentence recognition testing.

Certain aspects of the disclosure are directed to a kit comprising a composition comprising: a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition is formulated for intra-cochlear administration. Other aspects of the disclosure are directed to a kit comprising a composition comprising: a) a first rAAV vector genome comprising a first expression cassette comprising a promoter, a first coding sequence that encodes an N-terminal portion of an otoferlin protein positioned 3′ of the promoter, and a splicing donor signal sequence positioned at the 3′ end of the first coding sequence; and b) a second rAAV vector genome comprising a second expression cassette comprising a splicing acceptor signal sequence, a second coding sequence that encodes a C-terminal portion of an otoferlin protein positioned at the 3′ end of the splicing acceptor signal sequence, and a polyadenylation sequence at the 3′ end of the second coding sequence, wherein the composition is formulated for intra-cochlear administration.

In some aspects, the kit further comprises a pre-loaded syringe comprising the composition. In other aspects, the kit further comprises a vial comprising the composition.

Certain aspects of the disclosure are directed to a composition comprising: a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition comprises about 4.1E10-8.1E12 total vg.

In some aspects, the composition comprises about 4.1E10-4.1E12 total vg or about 8.1E10-8.1E12 total vg. In some aspects, the composition comprises about 4.1E10-4.1E12 total vg. In some aspects, the composition comprises about 4.1E11 total vg. In other aspects, the composition comprises about 8.1E10-8.1E12 total vg. In some aspects, the composition comprises about 8.1E11 total vg.

In some aspects, the composition comprises about 4.1E10-4.1E12 total vg/cochlea or about 8.1E10-8.1E12 total vg/cochlea. In some aspects, the composition comprises about 4.1E10-4.1E12 total vg/cochlea. In some aspects, the composition comprises about 4.1E11 total vg/cochlea. In other aspects, the composition comprises about 8.1E10-8.1E12 total vg/cochlea. In some aspects, the composition comprises about 8.1E11 total vg/cochlea.

In some aspects, the concentration of the composition comprises about 4.5E11-4.5E13 total vg/mL. In some aspects, the concentration of the composition comprises about 4.5E12 total vg/mL In other aspects, the concentration of the composition comprises about 9E11-9E13 total vg/mL. In some aspects, the composition comprises about 9E12 total vg/mL.

In some aspects, the composition comprises the first rAAV vector genome and the second rAAV vector genome in about a 1:1 ratio.

Certain aspects of the disclosure are directed to a composition comprising: a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition is formulated for intra-cochlear administration.

In some aspects, the composition comprises one or more pharmaceutically acceptable carriers, diluents, or excipients. In some aspects, the composition is formulated to comprise a synthetic perilymph solution.

In some aspects, the composition comprises one or more buffering agents and one or more surfactants. In some aspects, the buffering agents are selected from monopotassium phosphate, sodium phosphate dibasic, potassium chloride, sodium chloride, Tris HCl, Tris base, histidine, boric acid, citric acid, glycine, HEPES, and MOPS. In some aspects, the surfactant is selected from poloxamer 188, labrasol, tween, ethanol, pluronic F68, and polyethyelene glycol. In some aspects, the composition comprises monopotassium phosphate, sodium phosphate dibasic, potassium chloride, sodium chloride, and Poloxamer 188.

In some aspects, the formulation comprises: a) about 1.35-1.65 mM monopotassium phosphate; b) about 7.29-8.91 mM sodium phosphate dibasic; c) about 2.43-2.97 mM potassium chloride; d) about 154.8-189.2 mM sodium chloride; and e) about 0.0001%-0.01% Poloxamer 188. In some aspects, the formulation comprises: a) about 1.5 mM monopotassium phosphate; b) about 8.1 mM sodium phosphate dibasic; c) about 2.7 mM potassium chloride; d) about 172 mM sodium chloride; and e) about 0.001% Poloxamer 188.

In some aspects, the composition comprises about 4.1E10-8.1E12 total vg/cochlea. In some aspects, the composition comprises about 4.1E10-4.1E12 total vg/cochlea or about 8.1E10-8.1E12 total vg/cochlea. In some aspects, the composition comprises about 4.1E11 total vg/cochlea. In other aspects, the composition comprises about 8.1E11 total vg/cochlea.

In some aspects, the concentration of the composition comprises about 4.5E11-4.5E13 total vg/mL. In some aspects, the concentration of the composition comprises about 4.5E12 total vg/mL In other aspects, the concentration of the composition comprises about 9E11-9E13 total vg/mL. In some aspects, the concentration of the composition comprises about 9E12 total vg/mL.

In some aspects, the composition comprises the first rAAV vector genome and the second rAAV vector genome in about a 1:1 ratio.

In some aspects, the nucleic acid sequence comprising 5′ portion of the otoferlin gene has at least 80%, at least 85%, at least 90%, at least 91%, at least 92%, at least 93%, at least 94%, at least 95%, at least 96%, at least 97%, at least 98%, at least 99%, or 100% identity to SEQ ID NO: 101. In some aspects, the nucleic acid sequence comprising 3′ portion of the otoferlin gene has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 107.

In some aspects, the promoter is selected from a constitutive promoter, an inducible promoter, or a tissue-specific promoter. In some aspects, the promoter is a constitutive promoter. In some aspects, the constitutive promoter is selected from a CAG, CBA, or CMV promoter. In some aspects, the constitutive promoter is a CAG promoter.

In some aspects, the first rAAV vector comprises a splicing donor site and a recombinogenic sequence. In some aspects, the second rAAV vector comprises a splicing acceptor site, a recombinogenic sequence, and a polyadenylation sequence.

In some aspects, the splicing donor site has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 102. In some aspects, the splicing acceptor site has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 106.

In some aspects, the recombinogenic sequence has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 103.

In some aspects, the polyadenylation sequence is selected from a bovine growth hormone, human growth hormone, mouse-β-globin, mouse-α-globin, polyomavirus, SV40, or synthetic polyadenylation sequence. In some aspects, the polyadenylation is a bovine growth hormone polyadenylation sequence. In some aspects, the polyadenylation sequence has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 108.

In some aspects, the ITRs are selected from any one of AAV1, AAV2, AAV3, AAV4, AAV5, AAV6, AAV7, AAV8, AAV9, AAVrh8, AAVrh10, AAVrh39, AAVrh43, or Anc80 ITRs. In some aspects, the ITRs are AAV2 ITRs.

In some aspects, the first expression cassette has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 96.

In some aspects, the second expression cassette has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 105.

In some aspects, the first and second rAAV vectors are each encapsulated by an AAV capsid. In some aspects, the AAV capsid encapsulating the first rAAV vector is a serotype selected from any one of AAV1, AAV2, AAV3, AAV4, AAV5, AAV6, AAV7, AAV8, AAV9, AAVrh8, AAVrh10, AAVrh39, AAVrh43, or Anc80. In some aspects, the AAV capsid encapsulating the second rAAV vector is a serotype selected from any one of AAV1, AAV2, AAV3, AAV4, AAV5, AAV6, AAV7, AAV8, AAV9, AAVrh8, AAVrh10, AAVrh39, AAVrh43, or Anc80. In some aspects, the first rAAV vector is encapsulated by an Anc80 capsid and the second rAAV vector is encapsulated by an Anc80 capsid. In some aspects, the Anc80 capsids comprise the polypeptide sequence of SEQ ID NO: 109.

In some aspects, the composition is formulated for intra-cochlear administration. In some aspects, the composition comprises one or more pharmaceutically acceptable carriers, diluents, or excipients. In some aspects, the composition is formulated to comprise a synthetic perilymph solution.

In some aspects, the composition comprises one or more buffering agents and one or more surfactants. In some aspects, the buffering agents are selected from monopotassium phosphate, sodium phosphate dibasic, potassium chloride, sodium chloride, Tris HCl, Tris base, histidine, boric acid, citric acid, glycine, HEPES, and MOPS. In some aspects, the surfactant is selected from poloxamer 188, labrasol, tween, ethanol, pluronic F68, and polyethyelene glycol. In some aspects, the composition comprises monopotassium phosphate, sodium phosphate dibasic, potassium chloride, sodium chloride, and Poloxamer 188.

In some aspects, the formulation comprises: a) about 1.35-1.65 mM monopotassium phosphate; b) about 7.29-8.91 mM sodium phosphate dibasic; c) about 2.43-2.97 mM potassium chloride; d) about 154.8-189.2 mM sodium chloride; and e) about 0.0001%-0.01% Poloxamer 188. In some aspects, the formulation comprises: a) about 1.5 mM monopotassium phosphate; b) about 8.1 mM sodium phosphate dibasic; c) about 2.7 mM potassium chloride; d) about 172 mM sodium chloride; and e) about 0.001% Poloxamer 188.

In some aspects, the formulation is a sterile suspension. In some aspects, the formulation comprises sterile water.

In some aspects, the volume of the formulation is about 0.01 mL to 0.2 mL. In some aspects, the volume of the formulation is about 0.09 mL.

Certain aspects of the disclosure are directed to a method of treating hearing loss in a subject having a defective otoferlin gene, comprising administering a composition comprising: a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition comprises about 4.1E10-8.1E12 total vg, into the cochlea of the subject, wherein the first and second rAAV vectors are capable of constituting a polypeptide messenger RNA encoding a full length human otoferlin protein in the subject. Other aspects of the disclosure are directed to a method of treating hearing loss in a subject having a defective otoferlin gene, comprising administering a composition comprising: a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition is formulated for intra-cochlear administration, into the cochlea of the subject, wherein the first and second rAAV vectors are capable of constituting a polypeptide messenger RNA encoding a full length human otoferlin protein in the subject.

In some aspects, the defective otoferlin gene comprises a mutation that results in a decrease in the expression and/or activity of an otoferlin protein encoded by the gene. In some aspects, the method further comprises, prior to the administering step, determining that the subject has a defective otoferlin gene.

Certain aspects of the disclosure are directed to a method of treating hearing loss in a subject having biallelic otoferlin gene mutations, comprising administering a composition comprising: a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition comprises about 4.1E10-8.1E12 total vg, into the cochlea of the subject, wherein the first and second rAAV vectors are capable of constituting a polypeptide messenger RNA encoding a full length human otoferlin protein in the subject. Other aspects of the disclosure are directed to a method of treating hearing loss in a subject having biallelic otoferlin gene mutations, comprising administering a composition comprising: a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition is formulated for intra-cochlear administration, into the cochlea of the subject, wherein the first and second rAAV vectors are capable of constituting a polypeptide messenger RNA encoding a full length human otoferlin protein in the subject.

In some aspects, the method further comprises, prior to the administering step, determining that the subject has biallelic otoferlin gene mutations.

In some aspects, the the subject has a clinical presentation of bilateral profound sensorineural hearing loss. In some aspects, the subject has the clinical presentation of the bilateral profound sensorineural hearing loss when afebrile.

In some aspects, the method serves to mitigate or prevent secondary degeneration of one or more cochlear structures. In some aspects, the subject has preserved distortion product otoacoustic emissions (DPOAEs).

Certain aspects of the disclosure are directed to a method of expressing a recombinant full-length otoferlin protein in a mammalian cell, comprising administering a composition comprising: a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition comprises about 4.1E10-8.1E12 total vg, to the mammalian cell, wherein the first and second rAAV vectors are capable of constituting a polypeptide messenger RNA encoding a full length human otoferlin protein in the mammalian cell. Other aspects of the disclosure are directed to a method of expressing a recombinant full-length otoferlin protein in a mammalian cell, comprising administering a composition comprising: a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition is formulated for intra-cochlear administration, to the mammalian cell, wherein the first and second rAAV vectors are capable of constituting a polypeptide messenger RNA encoding a full length human otoferlin protein in the mammalian cell.

In some aspects, the mammalian cell is a cochlear cell. In some aspects, the mammalian cell is an inner ear hair cell. In some aspects, the subject is a mammal. In some aspects, the subject is a human.

In some aspects, the composition is administered in a single dose. In some aspects, the composition is administered in a plurality of doses. In some aspects, the composition is administered in 2, 3, 4, 5, 6, 7, 8, 9, or 10 doses.

In some aspects, a single dose comprises about 0.01 mL-0.2 mL. In some aspects, a single dose comprises about 0.09 mL.

In some aspects, the composition is administered as an injection to the round window membrane. In some aspects, the composition is administered in a single injection. In some aspects, the composition is administered in a plurality of injections. In some aspects, the composition is administered in 2, 3, 4, 5, 6, 7, 8, 9, or 10 injections.

In some aspects, the composition is administered through the use of a medical device. In some aspects, the composition is pre-loaded in the device. In some aspects, the device is a device as shown in FIGS. 2-5. In some aspects, the device is a microcatheter. In some aspects, the microcatheter is shaped such that it can enter the middle ear cavity via the external auditory canal and contact the end of the microcatheter with the round window membrane (RWM). In some aspects, a distal end of the microcatheter comprises at least one microneedle with diameter of between 10 and 1,000 microns. In some aspects, the at least one microneedle comprises a bent portion and an angled tip.

In some aspects, the composition is delivered at a controlled flow rate.

In some aspects, the subject is between 2 and 17 years of age.

In some aspects, the administration of the composition improves the subject's auditory brainstem response (ABR) threshold response, age-appropriate behavioral audiometry, tympanometry, and/or word/sentence recognition testing.

Certain aspects of the disclosure are directed to a kit comprising a composition comprising: a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition comprises about 4.1E10-8.1E12 total vg. Other aspects of the disclosure are directed to a kit comprising a composition comprising: a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition is formulated for intra-cochlear administration.

In some aspects, the composition is pre-loaded into a device. In some aspects, the device is a microcatheter. In some aspects, the microcatheter is shaped such that it can enter the middle ear cavity via the external auditory canal and contact the end of the microcatheter with the round window membrane (RWM). In some aspects, a distal end of the microcatheter is comprised of at least one microneedle with diameter of between 10 and 1,000 microns.

In some aspects, the kit further comprises a device. In some aspects, the device is a device described in any one of FIGS. 2-5. In some aspects, the device comprises a needle comprising a bent portion and an angled tip.

In some aspects, the kit further comprises a vial comprising the composition. In some aspects, the vial is a single-use vial. In some aspects, the kit further comprises a second vial comprising a diluent.

### DETAILED DESCRIPTION

Unless defined otherwise, all technical and scientific terms used herein have the same meaning as commonly understood by one of ordinary skill in the art to which the disclosure pertains.

The terms “a” and “an” refer to one or to more than one (i.e., at least one) of the grammatical object of the article. By way of example, “an element” encompasses one element and more than one element.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower), unless indicated otherwise.

The term “recombinant polypeptide” or “recombinant protein” refers to a polypeptide or protein which is generated using recombinant DNA technology, such as, for example, a polypeptide or protein expressed by a viral vector expression system. The term should also be construed to mean a polypeptide or protein which has been generated by the synthesis of a DNA molecule encoding the polypeptide or protein and which DNA molecule expresses a protein, or an amino acid sequence specifying the polypeptide, wherein the DNA or amino acid sequence has been obtained using recombinant DNA or amino acid sequence technology which is available and well known in the art.

The term “mutation in an otoferlin gene” refers to a modification in a wildtype otoferlin gene that results in the production of an otoferlin protein having one or more of: a deletion of one or more amino acids, one or more amino acid substitutions, and one or more amino acid insertions, as compared to the wildtype otoferlin protein, and/or results in a decrease in the expressed level of the encoded otoferlin protein in a mammalian cell as compared to the expressed level of the encoded otoferlin protein in a mammalian cell not having the mutation. In some aspects, a mutation can result in the production of an otoferlin protein having a deletion of one or more amino acids (e.g., 2, 3, 4, 5, 6, 7, 8, 9, 10, 11, 12, 13, 14, 15 16, 17, 18, 19, or 20 amino acids). In some aspects, the mutation can result in a frameshift in the otoferlin gene. The term “frameshift” is known in the art to encompass any mutation in a coding sequence that results in a shift in the reading frame of the coding sequence. In some aspects, a frameshift can result in a nonfunctional protein. In some aspects, a point mutation can be a nonsense mutation (i.e., result in a premature stop codon in an exon of the gene). A nonsense mutation can result in the production of a truncated protein (as compared to a corresponding wildtype protein) that may or may not be functional. In some aspects, the mutation can result in the loss (or a decrease in the level) of expression of otoferlin mRNA or otoferlin protein or both the mRNA and protein. In some aspects, the mutation can result in the production of an altered otoferlin protein having a loss or decrease in one or more biological activities (functions) as compared to a wildtype otoferlin protein.

In some aspects, the mutation is an insertion of one or more nucleotides into an otoferlin gene. In some aspects, the mutation is in a regulatory sequence of the otoferlin gene, i.e., a portion of the gene that is not coding sequence. In some aspects, a mutation in a regulatory sequence may be in a promoter or enhancer region and prevent or reduce the proper transcription of the otoferlin gene.

Modifications can be introduced into a nucleotide sequence by standard techniques known in the art, such as site-directed mutagenesis and PCR-mediated mutagenesis.

The term “conservative sequence modifications” refers to amino acid modifications that do not significantly affect or alter the binding characteristics of the antibody or antibody fragment containing the amino acid sequence. Such conservative modifications include amino acid substitutions, additions, and deletions. Modifications can be introduced into an antibody or antibody fragment of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, cysteine, and tryptophan), nonpolar side chains (e.g., alanine, valine, leucine, isoleucine, proline, phenylalanine, and methionine), beta-branched side chains (e.g., threonine, valine, and isoleucine), and aromatic side chains (e.g., tyrosine, phenylalanine, tryptophan, and histidine).

The term “encoding” refers to the inherent property of specific sequences of nucleotides in a polynucleotide, such as a gene, a cDNA, or an mRNA, to serve as templates for synthesis of a defined sequence of amino acids, in accordance with the genetic code. Thus, a gene, cDNA, or RNA encodes a protein if transcription and translation of mRNA corresponding to that gene, cDNA or RNA produces the protein. Both the coding strand, the nucleotide sequence of which is identical to the mRNA sequence and is usually provided in sequence listings, and the non-coding strand, used as the template for transcription, can be referred to as encoding the protein product.

The term “sequence identity” is used herein to mean a relationship between two or more amino acid (polypeptide or protein) sequences or two or more nucleic acid (polynucleotide) sequences, as determined by comparing the sequences. In certain aspects, sequence identity is calculated based on the full length of two given SEQ ID NO or on part thereof. Part thereof can mean at least 10%, 20%, 30%, 40%, 50%, 60%, 70%, 80%, 90%, or 100% of both SEQ ID NO, or any other specified percentage. The term “identity” can also mean the degree of sequence relatedness between amino acid or nucleic acid sequences, as the case may be, as determined by the match between strings of such sequences.

Unless otherwise specified, a “nucleotide sequence encoding an amino acid sequence” includes all nucleotide sequences that are degenerate versions of each other and thus encode the same amino acid sequence. A nucleotide sequence that encodes a protein may also include introns.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “expression” refers to the transcription and/or translation of a particular nucleotide sequence driven by a promoter.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “subject” is intended to include living organisms in which an immune response can be elicited (e.g., mammals, human). In some aspects, the subject is a rodent (e.g., a rat or mouse), a rabbit, a sheep, a dog, a cat, a horse, a non-human primate, or a human. In some aspects, the subject has or is at risk of developing non-syndromic deafness. In some aspects, the subject has been previously identified as having a mutation in an otoferlin gene. In some aspects, the subject has been identified as having a mutation in an otoferlin gene and has been diagnosed with non-syndromic sensorineural hearing loss. In some aspects, the subject has been identified as having non-syndromic sensorineural hearing loss.

The term “therapeutic” as used herein means a treatment. A therapeutic effect is obtained by reduction, suppression, remission, or eradication of a disease state.

The term “prophylaxis” as used herein means the prevention of, or protective treatment for, a disease or disease state. “Prevention” in this context includes reducing the likelihood the subject will experience the disease.

The term “effective amount” or “therapeutically effective amount” are used interchangeably herein, and refer to an amount of a compound, formulation, material, or composition as described herein effective to achieve a particular biological result. In some aspects, a therapeutically effective amount of a composition can result in an increase in the expression level of an active otoferlin protein (e.g., a wildtype, full-length otoferlin protein or of a variant of an otoferlin protein that has the desired activity) (e.g., as compared to the expression level prior to treatment with the composition). In some aspects, a therapeutically effective amount of a composition can result in an increase in the expression level of an active otoferlin protein (e.g., a wildtype, full-length otoferlin protein or active variant) in a target cell (e.g., a cochlear inner hair cell). In some aspects, a therapeutically effective amount of a composition can result in a different cellular localization of an active otoferlin protein (e.g., a wildtype, full-length otoferlin protein or an active variant) in a target cell (e.g., a cochlear inner hair cell). In some aspects, a therapeutically effective amount of a composition can result in an increase in the expression level of an active otoferlin protein (e.g., a wildtype, full-length otoferlin protein or active variant), and/or an increase in one or more activities of an otoferlin protein in a target cell (e.g., as compared to a reference level, such as the level(s) in a subject prior to treatment, the level(s) in a subject having a mutation in an otoferlin gene, or the level(s) in a subject or a population of subjects having non-syndromic sensorineural hearing loss).

The term “parenteral” administration of a composition includes, e.g., subcutaneous (s.c.), intravenous (i.v.), intramuscular (i.m.), or intrasternal injection, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

In some aspects of any of the nucleic acids described herein, the nucleic acid is DNA. In some aspects of any of the nucleic acids described herein, the nucleic acid is RNA.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

As used herein, “in vitro transcribed RNA” refers to RNA, preferably mRNA, that has been synthesized in vitro. Generally, the in vitro transcribed RNA is generated from an in vitro transcription vector. The in vitro transcription vector includes a template that is used to generate the in vitro transcribed RNA.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein including two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “active otoferlin protein” means a protein encoded by DNA that, if substituted for both wildtype alleles encoding full-length otoferlin protein in auditory hair cells (e.g., auditory inner hair cells) of what is otherwise a wildtype mammal, and if expressed in the auditory hair cells of that mammal, results in that mammal's having a level of hearing approximating the normal level of hearing of a similar mammal that is entirely wildtype. Non-limiting examples of active otoferlin proteins are full-length otoferlin proteins (e.g., any of the full-length otoferlin proteins described herein).

The term “vector” includes any genetic element, such as a plasmid, phage, transposon, cosmid, chromosome, artificial chromosome, virus, virion, etc., that is capable of replication when associated with the proper control elements and that can transfer gene sequences between cells. Thus, the term includes cloning and expression vehicles, as well as viral vectors. In some aspects, useful vectors are contemplated to be those vectors in which the nucleic acid segment to be transcribed is positioned under the transcriptional control of a promoter.

Vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide. Skilled practitioners will be capable of selecting suitable vectors and mammalian cells for making any of the nucleic acids described herein. A vector can, e.g., include sufficient cis-acting elements for expression; other elements for expression can be supplied by the host mammalian cell or in an in vitro expression system.

A “promoter” refers to a DNA sequence recognized by the synthetic machinery of the cell, or introduced synthetic machinery, required to initiate the specific transcription of a polynucleotide sequence (e.g., a gene).

The term “expression vector,” “construct,” or “expression cassette” means any type of genetic construct containing a nucleic acid in which part or all of the nucleic acid coding sequence is capable of being transcribed. In some aspects, expression includes transcription of the nucleic acid, for example, to generate a biologically-active polypeptide product or inhibitory RNA (e.g., shRNA, miRNA, miRNA inhibitor) from a transcribed gene.

The term “operably linked,” “operatively positioned,” “operatively linked,” “under control,” “under transcriptional control,” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “constitutive” promoter refers to a nucleotide sequence that, when operably linked with a nucleic acid encoding a protein (e.g., an otoferlin protein), causes RNA to be transcribed from the nucleic acid in a mammalian cell under most or all physiological conditions.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a promoter that is active only in certain specific cell types and/or tissues (e.g., transcription of a specific gene occurs only within cells expressing transcription regulatory proteins that bind to the tissue-specific promoter).

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, a “poly(A),” “poly(A) signal sequence,” “polyA sequence,” or “polyadenylation sequence” is a sequence that triggers the endonuclease cleavage of an mRNA and the additional of a series of adenosines to the 3′ end of the cleaved mRNA.

As used herein, the term “pharmaceutically acceptable carrier” includes buffering agents, surfactants, salts, solvents, dispersion media, coatings, antibacterial agents, antifungal agents, and the like that are compatible with pharmaceutical administration. Supplementary active compounds can also be incorporated into any of the compositions described herein.

As used herein, “carrier” includes any and all buffering agents, surfactants, salts, solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions.

A “viral genome” or “vector genome” or “viral vector” refers to a sequence that comprises one or more polynucleotide regions encoding or comprising a molecule of interest, e.g., a protein, a peptide, and a polynucleotide or a plurality thereof. Viral vectors are used to deliver genetic materials into cells. Viral vectors can be modified for specific applications. In some aspects, the delivery vectors comprises a viral vector selected from the group consisting of an adeno-associated viral (AAV) vector, an adenoviral vector, a lentiviral vector, or a retroviral vector.

The term “adeno-associated virus vector” or “AAV vector” as used herein refers to any vector which comprises or derives from components of an adeno-associated vector and is suitable to infect mammalian cells, preferably human cells. The term AAV vector typically designates an AAV-type viral particle or virion comprising a payload. The AAV vector can be derived from various serotypes, including combinations of serotypes (i.e., “pseudotyped” AAV) or from various genomes (e.g., single stranded or self-complementary). In addition, the AAV vector can be replication defective and/or targeted. As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh8, AAVrh10, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV. See, e.g., FIELDS et al. VIROLOGY, volume 2, chapter 69 (4th ed., Lippincott-Raven Publishers). In some aspects, an “AAV vector” includes a derivative of a known AAV vector. In some aspects, an “AAV vector” includes a modified or an artificial AAV vector. The terms “AAV genome” and “AAV vector” can be used interchangeably.

As used herein, a “recombinant AAV particle” or “rAAV particle” is an AAV virus that comprises a capsid protein and an AAV vector having at least one payload region and at least one inverted terminal repeat (ITR) region.

“Serotype” with respect to vector or virus capsid is defined by a distinct immunological profile based on the capsid protein sequences and capsid structure.

The term “ratio” refers to the comparison of two or more numbers that indicates their quantitative relation to each other. In some aspects, a ratios can be used to compare two portions within a whole or total amount.

As used herein, the term “vector ratio” refers to the amount, in vector genomes (vg), of one AAV vector compared to the amount, in vector genomes, of another AAV vector.

### Otoferlin

The human OTOF gene encodes otoferlin, which is a protein that, in some aspects, plays a critical role in priming, fusion, and/or replenishing of synaptic vesicles of inner hair cell synapses during sound encoding. To date, several hundred mutations in the human OTOF gene have been identified to cause profound prelingual deafness DFNB9. Such mutations are the cause of deafness in 2-8% of people that are born with an autosomal, recessively inherited, non-syndromic deafness in different populations (Rodriguez-Ballesteros et al. (2008) Hum. Mutat. 29 823-831; Choi et al. (2009) Clinical Genetics 75 237-243; Duman et al. (2011) Genet Test Mol Biomarkers 15 29-33; Varga et al. (2006) J Med Genet 43 576-581; Iwasa et al. (2013) BMC Med. Genet. 14 95. Biallelic otoferlin gene mutations cause localized, synaptic transmission defects between hair cells and the auditory nerve. Otoferlin enables sensory cells to release neurotransmitters in response to stimulation by sound to activate auditory neurons and those neurons carry electronically encoded acousting information to the brain to produce “hearing.” When biallelic mutations in OTOF are present, that transmission is impaired and, as a result, a majority of subjects have congenital, bilateral severe to profound sensorineural hearing loss. For example, two substitutions in exon 15 at positions 490 and 515 in the conserved C2C domain of otoferlin cause DFNB9 (Mirqhomizadeh et al. (2002) Neurobiol. Dis. 10 (2): 157-164). Migliosi et al. found a novel mutation Q829X in OTOF in Spanish subjects with prelingual non-syndromic hearing loss (Migliosi et al. (2002) J. Med. Genet. 39 (7): 502-506).

Additional exemplary mutations in a otoferlin gene detected in subjects having hearing loss and methods of sequencing a nucleic acid encoding otoferlin are described in, e.g., Rodriguez-Ballesteros et al. (2003) Hum Mutat. 22:451-456; Wang et al. (2010) BMC Med Genet. 11:79; Yildirim-Baylan et al. (2014) Int. J. Pediatr. Otorhinolaryngol 78:950-953; Choi et al. (2009) Clin. Genet. 75 (3): 237-243; and Marlin et al. (2010) Biochem Biophys Res Commun 394:737-742.

Otoacoustic emissions (e.g., distortion product otoacoustic emissions (DPOAEs)) from DFNB9 subjects are normal, at least for the first decade of life, indicating morphological integrity of the inner ear and proper function of outer hair cells. Accordingly, in some aspects, the subject has preserved DPOAEs. Apart from the lack of synaptic transmission and the subsequent loss of synapses, the morphology and physiology of the inner ear remains preserved in DFNB9, at least during the first decade in life in humans. Accordingly, in some aspects, restoration of OTOF and/or otoferlin function may serve to mitigate or prevent secondary degeneration of one or more cochlear structures.

Studies in mouse models revealed that synapses are structurally normal, and IHCs preserve normal synapse numbers in mice within the first postnatal week. Between P6 and P15, about half of the synapses get lost (Roux et al. (2006) Cell 127 277-289). Animal models allowed studying the effect of mutations in otoferlin on synaptic transmission by recording the change in plasma membrane capacitance after vesicle fusion and the activity in the auditory nerve. In otoferlin knock-out (Otof−/−) mice, almost no exocytosis could be triggered in IHCs by depolarization induced Ca2+ influx through voltage gated Ca2+ channels (Roux et al. (2006) Cell 127 277-289). In profoundly hearing impaired pachanga (OtofPga/Pga) mice with a random point mutation in the C2F domain, short (<10 ms) depolarizations of the IHCs elicited vesicle fusion of similar size as in wild type mice, however sustained stimulations uncovered a strong deficiency in replenishing vesicles to the readily releasable pool (Pangrsic et al. (2010) Nat. Neurosci. 13 869-876). The p.Ile515Thr mutation, found in human subjects with only mildly elevated hearing thresholds but a severe reduction in speech understanding and a temperature-dependent deafening (Varga et al. (2006) J Med Genet 43 576-581), uncovered an intermediate phenotype when studied in a mouse model (Strenzke et al. (2016) EMBO J. 35:2519-2535). Humans with bialellic pII515Thr mutations were shown to experience worsened hearing loss while febrile, with a return to pre-fever hearing function when afebrile (Varga et al. (2006) J Med Genet 43 576-581). Otof1515T/1515T mice showed a moderate elevation of hearing thresholds when assessed by ABR with a reduction in wave I amplitude, but normal auditory thresholds in behavioral tests and recordings of single auditory nerve units. Exocytosis of the RRP is again intact, but sustained exocytosis is reduced, although not as severe as in OtofPga/Pga. While in wild type mice at room temperature, during a sustained stimulus 750 vesicles can fuse per second at each active zone, this rate drops to 350 vesicles/s/active zone in Otof1515T/1515T mice and to 200 vesicles/s/active zone in OtofPga/Pga IHCs (Pangrsic et al. (2010) Nat. Neurosci. 13 869-876; Strenzke et al. (2016) EMBO J. 35 2519-2535). This correlated with lower otoferlin protein levels at the plasma membrane of IHCs, indicating that the amount of otoferlin scales with exocytosis and hearing (Strenzke et al. (2016) EMBO J. 35 2519-2535).

Methods of detecting mutations in a gene are well-known in the art. Non-limiting examples of such techniques include: real-time polymerase chain reaction (RT-PCR), PCR, sequencing, Southern blotting, and Northern blotting.

The OTOF gene encodes otoferlin, a protein that is involved in synaptic vesicle exocytosis in cochlear hair cells (see, e.g., Johnson and Chapman (2010) J. Cell Biol. 191 (1): 187-198; and Heidrych et al. (2008) Hum. Mol. Genet. 17:3814-3821).

The human OTOF gene is located on chromosome 2p23.3. It contains 48 exons encompassing ˜132 kilobases (kb) (NCBI Accession No. NG009937.1). The mRNA encoding the long-form of otoferlin expressed in the brain includes 48 exons (Yasunaga et al., Am. J. Hum. Genet. 67:591-600, 2000). Forward and reverse primers that can be used to amplify each of the 48 exons in the OTOF gene are described in Table 2 of Yasunaga et al., Am. J. Hum. Genet. 67:591-600, 2000. In some examples, the full-length OTOF protein is a full-length wildtype OTOF protein. The full-length wildtype OTOF protein expressed from the human OTOF gene is 1997 residues in length.

An exemplary human wildtype otoferlin protein is or includes the sequence of any one of SEQ ID NOs: 1-5. Isoform e of human otoferlin protein (SEQ ID NO: 5) is encoded by an mRNA that includes exon 48 and does not include exon 47 of the otoferlin gene (Yasunaga et al., Am. J. Hum. Genet. 67:591-600, 2000). In some aspects, the active otoferlin protein has the sequence of SEQ ID NO: 5, but is missing the 20 amino acids including the RXR motif identified in Strenzke et al., EMBO J. 35 (23): 2499-2615, 2016. Non-limiting examples of nucleic acids encoding a wildtype otoferlin protein are or include any one of SEQ ID NO: 7-11. As can be appreciated in the art, at least some or all of the codons in SEQ ID NO: 7-11 can be codon-optimized to allow for optimal expression in a non-human mammal or in a human. Orthologs of human otoferlin proteins are known in the art.

Human Otoferlin Protein cDNA Sequences:
Human canonical (long) isoform sequence (otoferlin protein) (SEQ ID NO: 1) (also called otoferlin isoform a) (NCBI Accession No. AAD26117.1)
Human Isoform 2 (short 1) (otoferlin protein) (SEQ ID NO: 2) (also called otoferlin isoform d) (NCBI Accession No. NP_919304.1)
Human Isoform 3 (short 2) (otoferlin protein) (SEQ ID NO: 3) (also called otoferlin isofom c) (NCBI Accession No. NP_919303.1)
Human Isoform 4 (short 3) (otoferlin protein) (SEQ ID NO: 4) (also called otoferlin isoform b) (NCBI Accession No. NP_004793.2)
Human Isoform 5 (short 4) (otoferlin protein) (SEQ ID NO: 5) (also called otoferlin isoform e) (NCBI Accession No. NP_001274418.1)
Complete cds (otoferlin cDNA) (www.ncbi.nlm.nih.gov/nuccore/AF107403.1) (SEQ ID NO: 6) (encodes the protein of SEQ ID NO: 1)
Human Otoferlin Transcript Variant 1 (www.ncbi.nlm.nih.gov/nuccore/NM_194248.2) (SEQ ID NO: 7) (encodes the protein of SEQ ID NO: 1)
Human Otoferlin Transcript Variant 2 (www.ncbi.nlm.nih.gov/nuccore/NM_004802.3) (SEQ ID NO: 8) (encodes the protein of SEQ ID NO: 4)
Human Otoferlin Transcript Variant 3 (www.ncbi.nlm.nih.gov/nuccore/NM_194322.2) (SEQ ID NO: 9) (encodes the protein of SEQ ID NO: 3)
Human Otoferlin Transcript Variant 4 (www.ncbi.nlm.nih.gov/nuccore/NM_194323.2) (SEQ ID NO: 10) (encodes the protein of SEQ ID NO: 2)
Human Otoferlin Transcript Variant 5 (www.ncbi.nlm.nih.gov/nuccore/NM_001287489.1) (SEQ ID NO: 11) (encodes the protein of SEQ ID NO: 5)

A non-limiting example of a human wildtype otoferlin genomic DNA sequence is SEQ ID NO: 12. The exons in SEQ ID NO: 12 are: nucleotide positions 5001-5206 (exon 1), nucleotide positions 25925-25983 (exon 2), nucleotide positions 35779-35867 (exon 3), nucleotide positions 44590-44689 (exon 4), nucleotide positions 47100-47281 (exon 5), nucleotide positions 59854-59927 (exon 6), nucleotide positions 61273-61399 (exon 7), nucleotide positions 61891-61945 (exon 8), nucleotide positions 68626-68757 (exon 9), nucleotide positions 73959-74021 (exon 10), nucleotide positions 74404-74488 (exon 11), nucleotide positions 79066-79225 (exon 12), nucleotide positions 80051-80237 (exon 13), nucleotide positions 81107-81293 (exon 14), nucleotide positions 82690-82913 (exon 15), nucleotide positions 83388-83496 (exon 16), nucleotide positions 84046-84226 (exon 17), nucleotide positions 84315-84435 (exon 18), nucleotide positions 85950-86050 (exon 19), nucleotide positions 86193-86283 (exon 20), nucleotide positions 86411-86527 (exon 21), nucleotide positions 86656-86808 (exon 22), nucleotide positions 87382-87571 (exon 23), nucleotide positions 87661-87785 (exon 24), nucleotide positions 88206-88340 (exon 25), nucleotide positions 89025-89186 (exon 26), nucleotide positions 89589-89708 (exon 27), nucleotide positions 90132-90293 (exon 28), nucleotide positions 90405-90567 (exon 29), nucleotide positions 91050-91180 (exon 30), nucleotide positions 92549-92578 (exon 31), nucleotide positions 92978-93106 (exon 32), nucleotide positions 95225-95291 (exon 33), nucleotide positions 96198-96334 (exon 34), nucleotide positions 96466-96600 (exon 35), nucleotide positions 96848-96985 (exon 36), nucleotide positions 97623-97750 (exon 37), nucleotide positions 97857-98027 (exon 38), nucleotide positions 98670-98830 (exon 39), nucleotide positions 99593-99735 (exon 40), nucleotide positions 100128-100216 (exon 41), nucleotide positions 101518-101616 (exon 42), nucleotide positions 101762-102003 (exon 43), nucleotide positions 102669-102847 (exon 44), nucleotide positions 102952-103052 (exon 45), nucleotide positions 103494-103691 (exon 46), nucleotide positions 105479-106496 (exon 47), and exon 48 (the sequence starting with CCGGCCCGAC; see also the description of this exon in Yasunaga et al., Am. J. Hum. Genet. 67:591-600, 2000).

The introns are located between each contiguous pair of exons in SEQ ID NO: 12, i.e., at nucleotide positions 100-5001 (intron 1), nucleotide 5207-25924 (intron 2), nucleotide positions 25984-35778 (intron 3), nucleotide positions 35868-44589 (intron 4), nucleotide positions 44690-47099 (intron 5), nucleotide positions 47282-59853 (intron 6), nucleotide positions 59928-61272 (intron 7), nucleotide positions 61400-61890 (intron 8), nucleotide positions 61946-68625 (intron 9), nucleotide positions 68758-73958 (intron 10), nucleotide positions 74022-74403 (intron 11), nucleotide positions 74489-79065 (intron 12), nucleotide positions 79226-80050 (intron 13), nucleotide positions 80238-81106 (intron 14), nucleotide positions 81294-82689 (intron 15), nucleotide positions 82914-83387 (intron 16), nucleotide positions 83497-84045 (intron 17), nucleotide positions 84227-84314 (intron 18), nucleotide positions 84436-85949 (intron 19), nucleotide positions 86051-86192 (intron 20), nucleotide positions 86284-86410 (intron 21), nucleotide positions 86528-86655 (intron 22), nucleotide positions 86809-87381 (intron 23), nucleotide positions 87572-87660 (intron 24), nucleotide positions 87786-88205 (intron 25), nucleotide positions 88341-89024 (intron 26), nucleotide positions 89187-89588 (intron 27), nucleotide positions 89709-90131 (intron 28), nucleotide positions 90294-90404 (intron 29), nucleotide positions 90568-91049 (intron 30), nucleotide positions 91181-92548 (intron 31), nucleotide positions 92579-92977 (intron 32), nucleotide positions 93107-95224 (intron 33), nucleotide positions 95292-96197 (intron 34), nucleotide positions 96335-96465 (intron 35), nucleotide positions 96601-96847 (intron 36), nucleotide positions 96986-97622 (intron 37), nucleotide positions 97751-97856 (intron 38), nucleotide positions 98028-98669 (intron 39), nucleotide positions 98831-99592 (intron 40), nucleotide positions 99736-100127 (intron 41), nucleotide positions 100217-101517 (intron 42), nucleotide positions 101617-101761 (intron 43), nucleotide positions 102004-102668 (intron 44), nucleotide positions 102848-102951 (intron 45), nucleotide positions 103053-103494 (intron 46), nucleotide positions 103692-105478 (intron 47), and nucleotide positions 106497-108496 (intron 48).

In some aspects, an otoferlin gene may be split into two or more segments between or within any appropriate exons and/or introns, where each segment is included in a different vector of the present disclosure. In some aspects, the otoferlin gene is split at exon 21, i.e., with exons 1 to (and through) 21 in a first vector and exons 22 to (and through) exon 48 in a second vector. In some such aspects the otoferlin segments in the first and second vectors are derived from an otoferlin cDNA sequence and lack introns, i.e., with exons 1 to (and through) 21 in a first vector and exons 22 to (and through) exon 48 in a second vector, each vector lacking otoferlin introns. In some aspects, an otoferlin gene may be split at one or more other exons and/or introns as long as, when combined with all other components of a vector, the packaging capacity of the vector is not exceeded.
Human Otoferlin Gene Sequence (ncbi.nlm.nih.gov/nuccore/224465243) (SEQ ID NO: 12)
Mouse Otoferlin Protein (SEQ ID NO: 13) (NCBI Accession No. NP_001300696.1)
Mouse Otoferlin cDNA (SEQ ID NO: 14) (NCBI Accession No. NM_001313767.1)
Mouse Otoferlin Gene Sequence (www.ncbi.nlm.nih.gov/gene/83762) (SEQ ID NO: 15) Accession: NC_000071 REGION: complement (30367066 . . . 30462730) GPC_000000778; NCBI Reference Sequence: NC_000071.6
Zebrafish Otoferlin A Gene Sequence (www.ncbi.nlm.nih.gov/gene/557476) (SEQ ID NO: 16) ACCESSION NC_007131 REGION: 31173357 . . . 31310109 GPC_000001574
NCBI Reference Sequence: NC_007131.7
Rhesus Monkey Otoferlin Gene Sequence (www.ncbi.nlm.nih.gov/gene/696717) (SEQ ID NO: 17) ACCESSION NC 027905 REGION: complement (26723411 . . . 26826586) GPC_000002105 NCBI Reference Sequence: NC_027905.1
Dog Otoferlin Gene Sequence (www.ncbi.nlm.nih.gov/gene/607961) (SEQ ID NO: 18)
ACCESSION NC_006599 REGION: complement (20518502 . . . 20619461) GPC_000000676 NCBI Reference Sequence: NC_006599.3
Chimpanzee Otoferlin Gene Sequence (www.ncbi.nlm.nih.gov/gene/459083) (SEQ ID NO: 19) ACCESSION NC 006469 REGION: complement (27006052 . . . 27107747) GPC_000002338 NCBI Reference Sequence: NC 006469.4
Rat Otoferlin Protein (SEQ ID NO: 20)
Zebrafish Otoferlin Protein (SEQ ID NO: 21)
Cow Otoferlin Protein (SEQ ID NO: 22)
Baboon Otoferlin Protein (SEQ ID NO: 23)

In some aspects, a first vector comprises a 5′ portion of OTOF cDNA, having a nucleic acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 94. In some aspects, the first vector comprises a 5′ portion of OTOF cDNA having the sequence of SEQ ID NO: 94.

In some aspects, a second vector comprises a 3′ portion of OTOF cDNA, having a nucleic acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 95. In some aspects, the second vector comprises a 5′ portion of OTOF cDNA having the sequence of SEQ ID NO: 95.

5′mOTOF DNA Sequence
(SEQ ID NO: 94)
ATGGCCCTGATTGTTCACCTCAAGACTGTCTCAGAGCTCCGAGGCAAAGG
TGACCGGATTGCCAAAGTCACTTTCCGAGGGCAGTCTTTCTACTCCCGGG
TCCTGGAGAACTGCGAGGGTGTGGCTGACTTTGATGAGACGTTCCGGTGG
CCAGTGGCCAGCAGCATCGACCGGAATGAAGTGTTGGAGATTCAGATTTT
CAACTACAGCAAAGTCTTCAGCAACAAGCTGATAGGGACCTTCTGCATGG
TGCTGCAGAAAGTGGTGGAGGAGAATCGGGTAGAGGTGACCGACACGCTG
ATGGATGACAGCAATGCTATCATCAAGACCAGCCTGAGCATGGAGGTCCG
GTATCAGGCCACAGATGGCACTGTGGGCCCCTGGGATGATGGAGACTTCC
TGGGAGATGAATCCCTCCAGGAGGAGAAGGACAGCCAGGAGACAGATGGG
CTGCTACCTGGTTCCCGACCCAGCACCCGGATATCTGGCGAGAAGAGCTT
TCGCAGAGCGGGAAGGAGTGTGTTCTCGGCCATGAAACTCGGCAAAACTC
GGTCCCACAAAGAGGAGCCCCAAAGACAAGATGAGCCAGCAGTGCTGGAG
ATGGAGGACCTGGACCACCTAGCCATTCAGCTGGGGGATGGGCTGGACCC
TGACTCCGTGTCTCTAGCCTCGGTCACCGCTCTCACCAGCAATGTCTCCA
ACAAACGGTCTAAGCCAGATATTAAGATGGAGCCCAGTGCTGGAAGGCCC
ATGGATTACCAGGTCAGCATCACAGTGATTGAGGCTCGGCAGCTGGTGGG
CTTGAACATGGACCCTGTGGTGTGTGTGGAGGTGGGTGATGACAAGAAAT
ACACGTCAATGAAGGAGTCCACAAACTGCCCTTACTACAACGAGTACTTT
GTCTTCGACTTCCATGTCTCTCCTGATGTCATGTTTGACAAGATCATCAA
GATCTCGGTTATCCATTCTAAGAACCTGCTTCGGAGCGGCACCCTGGTGG
GTTCCTTCAAAATGGATGTGGGGACTGTGTATTCCCAGCCTGAACACCAG
TTCCATCACAAATGGGCCATCCTGTCAGACCCCGATGACATCTCTGCTGG
GTTGAAGGGTTATGTAAAGTGTGATGTCGCTGTGGTGGGCAAGGGAGACA
ACATCAAGACACCCCACAAGGCCAACGAGACGGATGAGGACGACATTGAA
GGGAACTTGCTGCTCCCCGAGGGCGTGCCCCCCGAACGGCAGTGGGCACG
GTTCTATGTGAAAATTTACCGAGCAGAGGGACTGCCCCGGATGAACACAA
GCCTCATGGCCAACGTGAAGAAGGCGTTCATCGGTGAGAACAAGGACCTC
GTCGACCCCTATGTGCAAGTCTTCTTTGCTGGACAAAAGGGCAAAACATC
AGTGCAGAAGAGCAGCTATGAGCCGCTATGGAATGAGCAGGTCGTCTTCA
CAGACTTGTTCCCCCCACTCTGCAAACGCATGAAGGTGCAGATCCGGGAC
TCTGACAAGGTCAATGATGTGGCCATCGGCACCCACTTCATCGACCTGCG
CAAGATTTCCAACGATGGAGACAAAGGCTTCCTGCCTACCCTCGGTCCAG
CCTGGGTGAACATGTACGGCTCCACGCGCAACTACACACTGCTGGACGAG
CACCAGGACTTGAATGAAGGCCTGGGGGAGGGTGTGTCCTTCCGGGCCCG
CCTCATGTTGGGACTAGCTGTGGAGATCCTGGACACCTCCAACCCAGAGC
TCACCAGCTCCACGGAGGTGCAGGTGGAGCAGGCCACGCCTGTCTCGGAG
AGCTGCACAGGGAGAATGGAAGAATTTTTTCTATTTGGAGCCTTCTTGGA
AGCCTCAATGATTGACCGGAAAAATGGGGACAAGCCAATTACCTTTGAGG
TGACCATAGGAAACTACGGCAATGAAGTCGATGGTATGTCCCGGCCCCTG
AGGCCTCGGCCCCGGAAAGAGCCTGGGGATGAAGAAGAGGTAGACCTGAT
TCAGAACTCCAGTGACGATGAAGGTGACGAAGCCGGGGACCTGGCCTCGG
TGTCCTCCACCCCACCTATGCGGCCCCAGATCACGGACAGGAACTATTTC
CACCTGCCCTACCTGGAGCGCAAGCCCTGCATCTATATCAAGAGCTGGTG
GCCTGACCAGAGGCGGCGCCTCTACAATGCCAACATCATGGATCACATTG
CTGACAAGCTGGAAGAAGGCCTGAATGATGTACAGGAGATGATCAAAACG
GAGAAGTCCTACCCGGAGCGCCGCCTGCGGGGTGTGCTAGAGGAACTCAG
CTGTGGCTGCCACCGCTTCCTCTCCCTCTCGGACAAGGACCAGGGCCGCT
CGTCCCGCACCAGGCTGGATCGAGAGCGTCTTAAGTCCTGTATGAGGGAG
TTGGAGAGCATGGGACAGCAGGCCAAGAGCCTGAGGGCTCAGGTGAAGCG
GCACACTGTTCGGGACAAGCTGAGGTCATGCCAGAACTTTCTGCAGAAGC
TACGCTTCCTGGCGGATGAG
3′mOTOF DNA Sequence
(SEQ ID NO: 95)
CCCCAGCACAGCATTCCGGATGTGTTCATTTGGATGATGAGCAACAACAA
ACGTATCGCCTATGCCCGCGTGCCTTCCAAAGACCTGCTCTTCTCCATCG
TGGAGGAGGAACTGGGCAAGGACTGCGCCAAAGTCAAGACCCTCTTCCTG
AAGCTGCCAGGGAAGAGGGGCTTCGGCTCGGCAGGCTGGACAGTACAGGC
CAAGCTGGAGCTCTACCTGTGGCTGGGCCTCAGCAAGCAGCGAAAGGACT
TCCTGTGTGGTCTGCCCTGTGGCTTCGAGGAGGTCAAGGCAGCCCAAGGC
CTGGGCCTGCATTCCTTTCCGCCCATCAGCCTAGTCTACACCAAGAAGCA
AGCCTTCCAGCTCCGAGCACACATGTATCAGGCCCGAAGCCTCTTTGCTG
CTGACAGCAGTGGGCTCTCTGATCCCTTTGCCCGTGTCTTCTTCATCAAC
CAGAGCCAATGCACTGAGGTTCTAAACGAGACACTGTGTCCCACCTGGGA
CCAGATGCTGGTATTTGACAACCTGGAGCTGTACGGTGAAGCTCACGAGT
TACGAGATGATCCCCCCATCATTGTCATTGAAATCTACGACCAGGACAGC
ATGGGCAAAGCCGACTTCATGGGCCGGACCTTCGCCAAGCCCCTGGTGAA
GATGGCAGATGAAGCATACTGCCCACCTCGCTTCCCGCCGCAGCTTGAGT
ACTACCAGATCTACCGAGGCAGTGCCACTGCCGGAGACCTACTGGCTGCC
TTCGAGCTGCTGCAGATTGGGCCATCAGGGAAGGCTGACCTGCCACCCAT
CAATGGCCCAGTGGACATGGACAGAGGGCCCATCATGCCTGTGCCCGTGG
GAATCCGGCCAGTGCTCAGCAAGTACCGAGTGGAGGTGCTGTTCTGGGGC
CTGAGGGACCTAAAGAGGGTGAACCTGGCCCAGGTGGACCGACCACGGGT
GGACATCGAGTGTGCAGGAAAGGGGGTACAATCCTCCCTGATTCACAATT
ATAAGAAGAACCCCAACTTCAACACGCTGGTCAAGTGGTTTGAAGTGGAC
CTCCCGGAGAATGAGCTCCTGCACCCACCCTTGAACATCCGAGTGGTAGA
TTGCCGGGCCTTTGGACGATACACCCTGGTGGGTTCCCACGCAGTCAGCT
CACTGAGGCGCTTCATCTACCGACCTCCAGACCGCTCAGCCCCCAACTGG
AACACCACAGGGGAGGTTGTAGTAAGCATGGAGCCTGAGGAGCCAGTTAA
GAAGCTGGAGACCATGGTGAAACTGGATGCGACTTCTGATGCTGTGGTCA
AGGTGGATGTGGCTGAAGATGAGAAGGAAAGGAAGAAGAAGAAAAAGAAA
GGCCCGTCAGAGGAGCCAGAGGAGGAAGAGCCCGATGAGAGCATGCTGGA
TTGGTGGTCCAAGTACTTCGCCTCCATCGACACAATGAAGGAGCAACTTC
GACAACATGAGACCTCTGGAACTGACTTGGAAGAGAAGGAAGAGATGGAA
AGCGCTGAGGGCCTGAAGGGACCAATGAAGAGCAAGGAGAAGTCCAGAGC
TGCAAAGGAGGAGAAAAAGAAGAAAAACCAGAGCCCTGGCCCTGGCCAGG
GATCGGAGGCTCCTGAGAAGAAGAAAGCCAAGATCGATGAGCTTAAGGTG
TACCCCAAGGAGCTGGAATCGGAGTTTGACAGCTTTGAGGACTGGCTGCA
CACCTTCAACCTGTTGAGGGGCAAGACGGGAGATGATGAGGATGGCTCCA
CAGAGGAGGAGCGCATAGTAGGCCGATTCAAGGGCTCCCTCTGTGTGTAC
AAAGTGCCACTCCCAGAAGATGTATCTCGAGAAGCTGGCTATGATCCCAC
CTATGGAATGTTCCAGGGCATCCCAAGCAATGACCCCATCAATGTGCTGG
TCCGAATCTATGTGGTCCGGGCCACAGACCTGCACCCGGCCGACATCAAT
GGCAAAGCTGACCCCTATATTGCCATCAAGTTAGGCAAGACCGACATCCG
AGACAAGGAGAACTACATCTCCAAGCAGCTCAACCCTGTGTTTGGGAAGT
CCTTTGACATTGAGGCCTCCTTCCCCATGGAGTCCATGTTGACAGTGGCC
GTGTACGACTGGGATCTGGTGGGCACTGATGACCTCATCGGAGAAACCAA
GATTGACCTGGAAAACCGCTTCTACAGCAAGCATCGCGCCACCTGCGGCA
TCGCACAGACCTATTCCATACATGGCTACAATATCTGGAGGGACCCCATG
AAGCCCAGCCAGATCCTGACACGCCTCTGTAAAGAGGGCAAAGTGGACGG
CCCCCACTTTGGTCCCCATGGGAGAGTGAGGGTTGCCAACCGTGTCTTCA
CGGGGCCTTCAGAAATAGAGGATGAGAATGGTCAGAGGAAGCCCACAGAT
GAGCACGTGGCACTGTCTGCTCTGAGACACTGGGAGGACATCCCCCGGGT
GGGCTGCCGCCTTGTGCCGGAACACGTGGAGACCAGGCCGCTGCTCAACC
CTGACAAGCCAGGCATTGAGCAGGGCCGCCTGGAGCTGTGGGTGGACATG
TTCCCCATGGACATGCCAGCCCCTGGGACACCTCTGGATATATCCCCCAG
GAAACCCAAGAAGTACGAGCTGCGGGTCATCGTGTGGAACACAGACGAGG
TGGTCCTGGAAGACGATGATTTCTTCACGGGAGAGAAGTCCAGTGACATT
TTTGTGAGGGGGTGGCTGAAGGGCCAGCAGGAGGACAAACAGGACACAGA
TGTCCACTATCACTCCCTCACGGGGGAGGGCAACTTCAACTGGAGATACC
TCTTCCCCTTCGACTACCTAGCGGCCGAAGAGAAGATCGTTATGTCCAAA
AAGGAGTCTATGTTCTCCTGGGATGAGACGGAGTACAAGATCCCTGCGCG
GCTCACCCTGCAGATCTGGGACGCTGACCACTTCTCGGCTGACGACTTCC
TGGGGGCTATCGAGCTGGACCTGAACCGGTTCCCGAGGGGCGCTAAGACA
GCCAAGCAGTGCACCATGGAGATGGCCACCGGGGAGGTGGACGTACCCCT
GGTTTCCATCTTTAAACAGAAACGTGTCAAAGGCTGGTGGCCCCTCCTGG
CCCGCAATGAGAATGATGAGTTTGAGCTCACAGGCAAAGTGGAGGCGGAG
CTACACCTACTCACGGCAGAGGAGGCAGAGAAGAACCCTGTGGGCCTGGC
TCGCAATGAACCTGATCCCCTAGAAAAACCCAACCGGCCTGACACGGCAT
TCGTCTGGTTCCTGAACCCACTCAAATCTATCAAGTACCTCATCTGCACC
CGGTACAAGTGGCTGATCATCAAGATCGTGCTGGCGCTGCTGGGGCTGCT
CATGCTGGCCCTCTTCCTTTACAGCCTCCCAGGCTACATGGTCAAGAAGC
TCCTAGGGGCCTGA

Some aspects of any of the compositions described herein can include a first vector including the coding sequence of SEQ ID NO: 94 (or include a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 94). Some aspects of any of the compositions described herein can include a second vector including the coding sequence of SEQ ID NO: 95 (or include a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 95).

Some aspects of any of the compositions described herein can include a first rAAV vector with a 5′ OTOF coding region that comprises exons 1 to (and through) 21 of OTOF cDNA. In some aspects, the compositions comprises a first rAAV vector that comprises the nucleotide sequence of SEQ ID NO: 101 (or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the nucleotide sequence of SEQ ID NO: 101). In some aspects, the compositions comprise a first rAAV vector that comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the nucleotide sequence of SEQ ID NO: 101, and encodes the same amino acid sequence as encoded by SEQ ID NO: 101.

In some aspects, a composition of the present disclosure includes a first rAAV vector that comprises a codon optimized version of SEQ ID NO: 101, i.e., a nucleotide sequence that encodes the same amino acid sequence as encoded by SEQ ID NO: 101 but with codons that have been optimized for expression in a particular cell type, e.g., a mammalian cell, e.g., a human cell. In some aspects the first vector does not include any other portion of an OTOF gene. In some aspects the first vector does not include any other portion of OTOF cDNA.

In some aspects, the compositions comprise a second rAAV vector with a 3′ OTOF coding region that comprises exons 22 to (and through) exon 48 of OTOF cDNA. In some aspects, the compositions comprise a second rAAV vector that comprises the nucleotide sequence of SEQ ID NO: 107 (or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the nucleotide sequence of SEQ ID NO: 107). In some aspects, the compositions comprise a second rAAV vector that comprises a nucleotide sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the nucleotide sequence of SEQ ID NO: 108, and encodes the same amino acid sequence as encoded by SEQ ID NO: 107.

In some aspects, a composition comprises a second rAAV vector that comprises a codon optimized version of SEQ ID NO: 107, i.e., a nucleotide sequence that encodes the same amino acid sequence as encoded by SEQ ID NO: 107 but with codons that have been optimized for expression in a particular cell type, e.g., a mammalian cell, e.g., a human cell. In some aspects, the second rAAV vector does not include any other portion of an OTOF gene. In some aspects, the second rAAV vector does not include any other portion of OTOF cDNA.

One skilled in the art would appreciate that amino acids that are not conserved between wildtype otoferlin proteins from different species can be mutated without losing activity, while those amino acids that are conserved between wildtype otoferlin proteins from different species should not be mutated as they are more likely (than amino acids that are not conserved between different species) to be involved in activity.

### ITRs

In some aspects, the AAV vector comprises 5′ and 3′ inverted terminal repeats (ITRs). In some aspects, the rAAV vector comprises 5′ and 3′ inverted terminal repeats. In some aspects, the AAV sequences of the vector comprises the cis-acting 5′ and 3′ inverted terminal repeat sequences (See, e.g., B. J. Carter, in “Handbook of Parvoviruses”, ed., P. Tijsser, CRC Press, pp. 155 168 (1990)). In some aspects, the ITR sequences are about 145 nt in length. In some aspects, substantially the entire sequences encoding the ITRs are used in the molecule, although some degree of minor modification of these sequences is permissible. The ability to modify these ITR sequences is within the skill of the art. (See, e.g., texts such as Sambrook et al. “Molecular Cloning. A Laboratory Manual”, 2d ed., Cold Spring Harbor Laboratory, New York (1989); and K. Fisher et al., J Virol., 70:520 532 (1996)). An example of such a molecule employed in the present disclosure is a “cis-acting” plasmid containing the transgene, in which the selected transgene sequence and associated regulatory elements are flanked by 5′ and 3′ AAV ITR sequences. The AAV ITR sequences may be obtained from any known AAV, including presently identified mammalian AAV types. In some aspects, an ITR is or comprises 145 nucleotides.

In some aspects, the ITRs are AAV1, AAV2, AAV3, AAV4, AAV5, AAV6, AAV7, AAV8, AAV9, AAV10, AAV11, AAV12, or Anc80 ITRs. In some aspects, the ITRs are AAV2 ITRs. In some aspects, 5′ ITR has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 97. In some aspects, 5′ ITR has the sequence of SEQ ID NO: 97. In some aspects, 3′ ITR has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 104. In some aspects, 3′ ITR has the sequence of SEQ ID NO: 104.

In some aspects an ITR is a wild-type AAV2 ITR, e.g., 5′ ITR of SEQ ID NO: 97 and 3′ ITR of SEQ ID NO: 104. In some aspects an ITR is derived from a wild-type AAV2 ITR and includes one or more modifications, e.g., truncations, deletions, substitutions or insertions as is known in the art. In some aspects, an ITR comprises fewer than 145 nucleotides, e.g., 127, 130, 134 or 141 nucleotides. For example, in some aspects, an ITR comprises 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143 144, or 145 nucleotides.

In some aspects, 5′ ITR has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 59. In some aspects, 3′ ITR has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 60.

In some aspects, 5′ AAV ITR sequence has the sequence of SEQ ID NO: 59. In some aspects, 3′ AAV ITR sequence has the sequence of SEQ ID NO: 60. In some aspects, vectors and/or constructs of the present disclosure comprise a 5′ AAV ITR and/or a 3′ AAV ITR. In some aspects, 5′ AAV ITR sequence has the sequence of SEQ ID NO: 97. In some aspects, 3′ AAV ITR sequence has the sequence of SEQ ID NO: 104. In some aspects, 5′ AAV ITR sequence is SEQ ID NO: 97 and 3′ AAV ITR sequence is SEQ ID NO: 104. In some aspects, 5′ and a 3′ AAV ITRs (e.g., SEQ ID NOs: 97 and 104) flank a portion of a transgene and/or construct comprising a portion of OTOF (e.g., SEQ ID NO: 101 or 107).

In some aspects, the recombinant AAV vector comprises a nucleic acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 96. In some aspects, the recombinant AAV vector comprises a nucleic acid sequence with the sequence of SEQ ID NO: 96.

In some aspects, the recombinant AAV vector comprises a nucleic acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 105. In some aspects, the recombinant AAV vector comprises a nucleic acid sequence with the sequence of SEQ ID NO: 105.

### Promoters

In some aspects, the first recombinant adeno-associated viral (rAAV) vector comprises a first expression cassette comprising a promoter operably linked to nucleic acid sequence comprising a 5′ portion of an otoferlin gene. In some aspects, the first recombinant adeno-associated viral (rAAV) vector genome comprises a first expression cassette comprising a promoter operably linked to nucleic acid sequence comprising a 5′ portion of an otoferlin gene.

In some aspects, the promoter is selected from a constitutive promoter, an inducible promoter, or a tissue-specific promoter. In some aspects, the promoter is a constitutive promoter. In some aspects, the constitutive promoter is selected from a CAG, CBA, or CMV promoter. In some aspects, the constitutive promoter is a CAG promoter.

Non-limiting examples of promoters are described herein. Additional examples of promoters are known in the art.

In some aspects, a vector encoding an N-terminal portion of an otoferlin protein (e.g., a human otoferlin protein) can include a promoter and/or an enhancer. The vector encoding the N-terminal portion of the otoferlin protein can include any of the promoters and/or enhancers described herein or known in the art.

In some aspects, the promoter is an inducible promoter, a constitutive promoter, a mammalian cell promoter, a viral promoter, a chimeric promoter, an engineered promoter, a tissue-specific promoter, or any other type of promoter known in the art. In some aspects, the promoter is a RNA polymerase II promoter, such as a mammalian RNA polymerase II promoter. In some aspects, the promoter is a RNA polymerase III promoter, including, but not limited to, a H1 promoter, a human U6 promoter, a mouse U6 promoter, or a swine U6 promoter. The promoter will generally be one that is able to promote transcription in cochlear cells such as hair cells. In some examples, the promoter is a cochlea-specific promoter or a cochlea-oriented promoter.

A variety of promoters are known in the art that can be used herein. Non-limiting examples of promoters that can be used herein include: human elongation factor 1α-subunit (EF1a) (Liu et al. (2007) Exp. Mol. Med. 39 (2): 170-175; Accession No. J04617.1; Gill et al., Gene Ther. 8 (20): 1539-1546, 2001; Xu et al., Human Gene Ther. 12 (5): 563-573, 2001; Xu et al., Gene Ther. 8:1323-1332; Ikeda et al., Gene Ther. 9:932-938, 2002; Gilham et al., J. Gene Med. 12 (2): 129-136, 2010), cytomegalovirus (Xu et al., Human Gene Ther. 12 (5): 563-573, 2001; Xu et al., Gene Ther. 8:1323-1332; Gray et al., Human Gene Ther. 22:1143-1153, 2011), human immediate-early cytomegalovirus (CMV) (U.S. Pat. No. 5,168,062, Liu et al. (2007) Exp. Mol. Med. 39 (2): 170-175; Accession No. X17403.1 or KY490085.1), human ubiquitin C (UBC) (Gill et al., Gene Ther. 8 (20): 1539-1546, 2001; Qin et al., PLOS One 5 (5): e10611, 2010), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g. human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2κ b, HSP70, proliferin, tumor necrosis factor, thyroid stimulating hormone a gene, immunoglobulin light chain, T-cell receptor, HLA DQα and DQβ, interleukin-2 receptor, MHC class II, MHC class II HLA-DRα, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, Gibbon Ape Leukemia Virus (GALV) promoters, promoter of HNRPA2B1-CBX1 (UCOE) (Powell and Gray (2015) Discov. Med. 19 (102): 49-57; Antoniou et al., Human Gene Ther. 24 (4): 363-374, 2013), β-glucuronidase (GUSB) (Husain et al., Gene Ther. 16:927-932, 2009), chicken β-actin (CBA) (Liu et al. (2007) Exp. Mol. Med. 39 (2): 170-175; Stone et al. (2005) Mol. Ther. 11 (6): 843-848; Klein et al., Exp. Neurol. 176 (1): 66-74, 2002; Ohlfest et al., Blood 105:2691-2698, 2005; Gray et al., Human Gene Ther. 22:1143-1153, 2011), a human β-actin promoter (HBA) (Accession No. Y00474.1), murine myosin VIIA (musMyo7) (Boeda et al. (2001) Hum. Mol. Genet. 10 (15): 1581-1589; Accession No. AF384559.1), human myosin VIIA (hsMyo7) (Boeda et al. (2001) Hum. Mol. Genet. 10 (15): 1581-1589; Accession No. NG_009086.1), murine poly (ADP-ribose) polymerase 2 (musPARP2) (Ame et al. (2001) J. Biol. Chem. 276 (14): 11092-11099; Accession No. AF191547.1), human poly(ADP-ribose) polymerase 2 (hsPARP2) (Ame et al. (2001) J. Biol. Chem. 276 (14): 11092-11099; Accession No. X16612.1 or AF479321.1), acetylcholine receptor epsilon-subunit (AChε) (Duclert et al. (1993) PNAS 90 (7): 3043-3047; Accession No. S58221.1 or CR933736.12), Rous sarcoma virus (RSV) (Liu et al. (2007) Exp. Mol. Med. 39 (2): 170-175; Accession No. M77786.1), (GFAP) (Liu et al. (2007) Exp. Mol. Med. 39 (2): 170-175; Stone et al. (2005) Mol. Ther. 11 (6): 843-848; Accession No. NG_008401.1 or M67446.1), hAAT (Van Linthout et al., Human Gene Ther. 13 (7): 829-840, 2002; Cunningham et al., Mol. Ther. 16 (6): 1081-1088, 2008), and a CBA hybrid (CBh) (Gray et al. (2011) Hum. Gen. Therapy 22:1143-1153; Accession No. KF926476.1 or KC152483.1). Additional examples of promoters are known in the art. See, e.g., Lodish, Molecular Cell Biology, Freeman and Company, New York 2007. In some aspects, the promoter is the CMV immediate early promoter.

In some aspects, the promoter is a CAG promoter or a CAG/CBA promoter. In some aspects, a vector or construct of the present disclosure comprises a CAG promoter.

In some aspects, a CAG promoter comprises, in order from 5′ to 3′, the nucleotide sequences of SEQ ID NOs: 98, 99, and 100.

In some aspects, the CAG promoter comprises a CMV early enhancer element, a chicken beta actin (CBA) gene sequence, and a chimeric intron/3′ splice sequence from the rabbit beta globin gene.

In some aspects, the CMV early enhancer element comprises a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 98. In some aspects, the CMV early enhancer element has the sequence of SEQ ID NO: 98.

In some aspects, the CBA gene sequence has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 99. In some aspects, the CBA gene sequence has the sequence of SEQ ID NO: 99.

In some aspects, the chimeric intron/3′ splice sequence form the rabbit beta globin gene has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 100. In some aspects, the chimeric intron/3′ splice sequence form the rabbit beta globin gene has the sequence of SEQ ID NO: 100.

Examples of constitutive promoters include, without limitation, the CAG promoter, the retroviral Rous sarcoma virus (RSV) LTR promoter, the cytomegalovirus (CMV) promoter (see, e.g., Boshart et al. Cell 41:521-530, 1985), the SV40 promoter, the dihydrofolate reductase promoter, the beta-actin promoter, the phosphoglycerol kinase (PGK) promoter, and the EF1-alpha promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art.

Examples of inducible promoters regulated by exogenously supplied compounds include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al. Proc. Natl. Acad. Sci. U.S.A. 93:3346-3351, 1996), the tetracycline-repressible system (Gossen et al. Proc. Natl. Acad. Sci. U.S.A. 89:5547-5551, 1992), the tetracycline-inducible system (Gossen et al. Science 268:1766-1769, 1995, see also Harvey et al. Curr. Opin. Chem. Biol. 2:512-518, 1998), the RU486-inducible system (Wang et al. Nat. Biotech. 15:239-243, 1997) and Wang et al. Gene Ther. 4:432-441, 1997), and the rapamycin-inducible system (Magari et al. J. Clin. Invest. 100:2865-2872, 1997).

In some aspects, the tissue-specific promoter is active only in the inner ear. In some aspects, the tissue-specific promoter is active only in the cochlea. In some aspects, the tissue-specific promoter is active only in hair cells. In some aspects, the tissue-specific promoter is active only in inner hair cells.

Exemplary tissue-specific promoters include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter (Kugler et al., Virology 311:89-95, 2003; Hioki et al., Gene Ther. 14:872-882, 2007; Kuroda et al., J. Gene Med. 10:1163-1175, 2008), a creatine kinase (MCK) promoter (Wang et al., Gene Ther. 15:1489-1499, 2008; Talbot et al., Mol. Ther. 18:601-608, 2010; Katwal et al., Gene Ther. 20 (9): 930-938, 2013), a mammalian desmin (DES) promoter (Talbot et al., Mol. Ther. 18:601-608, 2010), a C5-12 promoter (Wang et al., Gene Ther. 15:1489-1499, 2008), an α-myosin heavy chain (a-MHC) promoter, a PDGF promoter (Patterna, Gene Ther. 7 (15): 1304-1311, 2000; Hioki et al., Gene Ther. 14:872-882, 2007), MecP2 promoter (Rastegar et al., PLOS One 4: e6810, 2009; Gray et al., Human Gene Ther. 22:1143-1153, 2011), CaMKII promoter (Hioki et al., Gene Ther. 14:872-882, 2007; Kuroda et al., J. Gene Med. 10:1163-1175, 2008), mGluR2 promoter (Brene et al., Eur. J. Eurosci. 12:1525-1533, 2000; Kuroda et al., J. Gene Med. 10:1163-1175, 2008), NFL promoter (Xu et al., Human Gene Ther. 12 (5): 563-573, 2001; Xu et al., Gene Ther. 8:1323-1332, 2001), NFH promoter (Xu et al., Human Gene Ther. 12 (5): 563-573, 2001; Xu et al., Gene Ther. 8:1323-1332, 2001), nε2 promoter (Xu et al., Human Gene Ther. 12 (5): 563-573, 2001; Xu et al., Gene Ther. 8:1323-1332, 2001), PPE promoter (Xu et al., Human Gene Ther. 12 (5): 563-573, 2001; Xu et al., Gene Ther. 8:1323-1332, 2001), Enk promoter (Xu et al., Human Gene Ther. 12 (5): 563-573, 2001; Xu et al., Gene Ther. 8:1323-1332, 2001), EAAT2 promoter (Su et al., Proc. Natl. Acad. Sci. U.S.A. 100:1955-1960, 2003; Kuroda et al., J. Gene Med. 10:1163-1175, 2008), GFAP promoter (Brenner et al., J. Neurosci. 14:1030-1037, 1994; Xu et al., Human Gene Ther. 12 (5): 563-573, 2001; Xu et al., Gene Ther. 8:1323-1332, 2001; Lee et al., Glia 56:481-493, 2008; Dirren et al., Human Gene Ther. 25:109-120, 2014), MBP promoter (Chen et al., Gene Ther. 5 (1): 50-58, 1998), or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor.alpha.-chain promoter, neuronal such as neuron-specific enolase (NSE) promoter (Andersen et al., Cell. Mol. Neurobiol., 13:503-15 (1993); Xu et al., Human Gene Ther. 12 (5): 563-573, 2001; Xu et al., Gene Ther. 8:1323-1332, 2001), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some aspects, the regulatory sequences impart tissue-specific gene expression capabilities. In some cases, the tissue-specific regulatory sequences bind tissue-specific transcription factors that induce transcription in a tissue-specific manner.

In some aspects, the tissue-specific promoter is a cochlea-specific promoter. In some aspects, the tissue-specific promoter is a cochlear hair cell-specific promoter. Non-limiting examples of cochlear hair cell-specific promoters include but are not limited to: a ATOH1 promoter, a POU4F3 promoter, a LHX3 promoter, a MYO7A promoter, a MYO6 promoter, a α9ACHR promoter, and a α10ACHR promoter.

In another aspect, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further aspect, other native expression control elements, such as enhancer elements, polyadenylation sites or Kozak consensus sequences may also be used to mimic the native expression.

### Enhancers

In some instances, a vector can include a promoter sequence and/or an enhancer sequence. The term “enhancer” refers to a nucleotide sequence that can increase the level of transcription of a nucleic acid encoding a protein of interest (e.g., an otoferlin protein). Enhancer sequences (50-1500 base pairs in length) generally increase the level of transcription by providing additional binding sites for transcription-associated proteins (e.g., transcription factors). In some aspects, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer, and a SV40 enhancer. An example of a CMV enhancer is described in, e.g., Boshart et al., Cell 41(2): 521-530, 1985.

In some aspects, the enhancer is a CMV early enhancer. In some aspects, the CMV early enhancer element comprises a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 98. In some aspects, the CMV early enhancer has the sequence of SEQ ID NO: 98.

### Poly(A) Sequences

In some aspects, any of the vectors provided herein can include a poly(A) sequence. Most nascent eukaryotic mRNAs possess a poly(A) tail at their 3′ end which is added during a complex process that includes cleavage of the primary transcript and a coupled polyadenylation reaction (see, e.g., Proudfoot et al., Cell 108:501-512, 2002). The poly(A) tail confers mRNA stability and transferability (Molecular Biology of the Cell, Third Edition by B. Alberts et al., Garland Publishing, 1994). In some aspects, the poly(A) sequence is positioned 3′ to the nucleic acid sequence encoding the C-terminus of the otoferlin protein.

Poly(A) sequences can be modified chemically or enzymatically to modulate mRNA functionality such as localization, stability or efficiency of translation.

There are several poly(A) signal sequences that can be used, including those derived from bovine growth hormone (bgh) (Woychik et al., Proc. Natl. Acad. Sci. U.S.A. 81 (13): 3944-3948, 1984; U.S. Pat. No. 5,122,458; Yew et al., Human Gene Ther. 8 (5): 575-584, 1997; Xu et al., Human Gene Ther. 12 (5): 563-573, 2001; Xu et al., Gene Ther. 8:1323-1332, 2001; Wu et al., Mol. Ther. 16 (2): 280-289, 2008; Gray et al., Human Gene Ther. 22:1143-1153, 2011; Choi et al., Mol. Brain 7:17, 2014), mouse-β-globin, mouse-α-globin (Orkin et al., EMBO J. 4 (2): 453-456, 1985; Thein et al., Blood 71 (2): 313-319, 1988), human collagen, polyoma virus (Batt et al., Mol. Cell Biol. 15 (9): 4783-4790, 1995), the Herpes simplex virus thymidine kinase gene (HSV TK), IgG heavy-chain gene polyadenylation signal (US 2006/0040354), human growth hormone (hGH) (Szymanski et al., Mol. Therapy 15 (7): 1340-1347, 2007; Ostegaard et al., Proc. Natl. Acad. Sci. U.S.A. 102 (8): 2952-2957, 2005), synthetic polyA (Levitt et al., Genes Dev. 3 (7): 1019-1025, 1989; Yew et al., Human Gene Ther. 8 (5): 575-584, 1997; Ostegaard et al., Proc. Natl. Acad. Sci. U.S.A. 102 (8): 2952-2957, 2005; Choi et al., Mol. Brain 7:17, 2014), HIV-1 upstream poly(A) enhancer (Schambach et al., Mol. Ther. 15 (6): 1167-1173, 2007), adenovirus (L3) upstream poly(A) enhancer (Schambach et al., Mol. Ther. 15 (6): 1167-1173, 2007), hTHGB upstream poly(A) enhancer (Schambach et al., Mol. Ther. 15 (6): 1167-1173, 2007), hC2 upstream poly(A) enhancer (Schambach et al., Mol. Ther. 15 (6): 1167-1173, 2007), the group consisting of SV40 poly(A) signal sequence, such as the SV40 late and early poly(A) signal sequence (Schek et al., Mol. Cell Biol. 12 (12): 5386-5393, 1992; Choi et al., Mol. Brain 7:17, 2014; Schambach et al., Mol. Ther. 15 (6): 1167-1173, 2007). In some aspects, the polyA sequence has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 68. In some aspects, the polyA sequence has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 76. In some aspects, the polyA sequence has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 77. A non-limiting example of a poly(A) signal sequence is SEQ ID NO: 68, 76, or 77.

The poly(A) signal sequence can be the sequence AATAAA. The AATAAA sequence may be substituted with other hexanucleotide sequences with homology to AATAAA which are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414).

In some aspects, the poly(A) signal sequence can be a synthetic polyadenylation site (see, e.g., the pCl-neo expression vector of Promega which is based on Levitt el al, Genes Dev. 3 (7): 1019-1025, 1989). In some aspects, the poly(A) signal sequence is the polyadenylation signal of soluble neuropilin-1 (sNRP) (AAATAAAATACGAAATG) (see, e.g., WO 05/073384).

In some aspects, a poly(A) sequence is a bovine growth hormone poly(A) sequence. In some aspects, the polyA sequence has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 108. In some such aspects, a bGH poly(A) sequence comprises or is the sequence of SEQ ID NO: 108. In some aspects, a vector or construct of the present disclosure comprises a boving growth hormone poly A sequence represented by SEQ ID NO: 108. Additional examples of poly(A) signal sequences are known in the art.

### Flanking Regions Untranslated Regions (UTRs)

In some aspects, any of the vectors described herein (e.g., any of the at least two different vectors) can include an untranslated region. In some aspects, a vector can includes a 5′ UTR or a 3′ UTR.

Untranslated regions (UTRs) of a gene are transcribed but not translated. The 5′ UTR starts at the transcription start site and continues to the start codon but does not include the start codon. The 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into any of the vectors, compositions, kits, or methods as described herein to enhance the stability of an otoferlin protein.

### Splice Donor and Splice Acceptor Sequences

In other aspects, non-UTR sequences may be incorporated into 5′ or 3′ UTRs. In some aspects, introns or portions of intron sequences may be incorporated into the flanking regions of the polynucleotides in any of the vectors, compositions, kits, and methods provided herein. Incorporation of intronic sequences may increase protein production as well as mRNA levels. An intron can be an intron from an otoferlin gene or can be an intron from a heterologous gene, e.g., a hybrid adenovirus/mouse immunoglobulin intron (Yew et al., Human Gene Ter. 8 (5): 575-584, 1997), an SV40 intron (Ostedgaard et al., Proc. Natl. Acad. Sci. U.S.A. 102 (8): 2952-2957, 2005), an MVM intron (Wu et al., Mol. Ther. 16 (2): 280-289, 2008), a factor IX truncated intron 1 (Wu et al., Mol. Ther. 16 (2): 280-289, 2008; Kurachi et al., J. Biol. Chem. 270 (10): 5276-5281, 1995), a chimeric 0-globulin splice donor/immunoglobulin heavy chain splice acceptor intron (Wu et al., Mol. Ther. 16 (2): 280-289, 2008; Choi et al., Mol. Brain 7:17, 2014), SV40 late splice donor/splice acceptor intron (19S/16S) (Yew et al., Human Gene Ther. 8 (5): 575-584, 1997), hybrid adenovirus spice donor/IgG splice acceptor (Choi et al., Mol. Brain 7:17, 1991; Huang and Gorman, Mol. Cell Biol. 10 (4): 1805-1810, 1990).

In some aspects, the splice donor has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 64. In some aspects, the splice donor has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 72. In some aspects, the splice donor has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 74. In some aspects, the splice donor has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 102. In some aspects, the splice donor has the sequence of SEQ ID NO: 102.

In some aspects, the splice acceptor has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 65. In some aspects, the splice acceptor has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 73. In some aspects, the splice acceptor has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 75. In some aspects, the splice acceptor has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 106. In some aspects, the splice acceptor has the sequence of SEQ ID NO: 106.

Non-limiting examples of a splice donor and splice acceptor sequences are SEQ ID NOs: 64 and 65, respectively; SEQ ID NOs: 72 and 73, respectively; SEQ ID NOs: 74 and 75, respectively, and SEQ ID NOs: 102 and 106, respectively.

In some aspects, the splice donor sequence has the sequence of SEQ ID NO: 102. In some aspects, a vector of construct of the present disclosure comprises a splice donor sequence of SEQ ID NO: 102. In some such aspects, the vector or construct comprising a splice donor sequence (e.g., SEQ ID NO: 102) also comprises a 5′ portion of the OTOF gene or OTOF cDNA (e.g., SEQ ID NO: 101) upstream of the splice donor sequence.

In some aspects, the first rAAV vector comprises a splice donor sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 102. In some aspects, the first rAAV vector comprises a first expression cassette comprising a splice donor sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 102.

In some aspects, the first rAAV vector genome comprises a splice donor sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 102. In some aspects, the first rAAV vector genome comprises a first expression cassette comprising a splice donor sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 102. In some aspects, the splice donor has the sequence of SEQ ID NO: 102.

In some aspects, the splice acceptor sequence has the sequence of SEQ ID NO: 106. In some aspects, a vector or construct of the present disclosure comprises a splice acceptor sequence of SEQ ID NO: 106. In some such aspects, the vector or construct comprising a splice acceptor sequence (e.g., SEQ ID NO: 106) also comprises a 3′ portion of the OTOF gene or OTOF cDNA (e.g., SEQ ID NO: 107) downstream of the splice acceptor sequence.

In some aspects, the second rAAV vector comprises a splice acceptor sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 106. In some aspects, the second rAAV vector comprises a second expression cassette comprising a splice acceptor sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 106.

In some aspects, the second rAAV vector genome comprises a splice acceptor sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 106. In some aspects, the second rAAV vector genome comprises a second expression cassette comprising a splice acceptor sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 106. In some aspects, the splice acceptor has the sequence of SEQ ID NO: 106.

### Recombinogenic Sequences

In some aspects, the vectors or constructs of the present disclosure comprise(s) one or more recombinogenic sequences. In some aspects, a recombinogenic sequence is or comprises a portion of a gene sequence. In some aspects, a recombinogenic sequence is derived from an alkaline phosphatase gene. In some aspects, a recombinogenic sequence is derived from an F1 phage. In some such aspects, a recombinogenic sequence is an AK sequence derived from an F1 phage.

In some aspects, the AK recombinogenic sequence has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 103. In some aspects, the AK recombinogenic sequence is SEQ ID NO: 103. In some aspects of a dual vector system of the present disclosure, each of two vectors comprises a recombinogenic sequence.

In some aspects, the first rAAV vector comprises an AK recombinogenic sequence has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 103. In some aspects, the first rAAV vector comprises an expression cassette comprising an AK recombinogenic sequence has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 103. In some aspects, the AK recombinogenic sequence has the sequence of SEQ ID NO: 103.

In some aspects, the first rAAV vector genome comprises an AK recombinogenic sequence has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 103. In some aspects, the first rAAV vector genome comprises an expression cassette comprising an AK recombinogenic sequence has a sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 103. In some aspects, the AK recombinogenic sequence has the sequence of SEQ ID NO: 103.

### Additional Sequences

In addition to the major elements identified above for the recombinant AAV vector, the vector also includes conventional control elements that are operably linked to the transgene in a manner which permits its transcription, translation and/or expression in a cell transfected with the plasmid vector or infected with the virus produced by the disclosure.

Any of the vectors provided herein can optionally include additional nucleotide sequences (“a stuffer sequence”) in order to optimize the total number of base pairs in the vector.

In some aspects, the stuffer sequence has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 54. In some aspects, the stuffer sequence has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 55. In some aspects, the stuffer sequence has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 56. In some aspects, the stuffer sequence has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 57. In some aspects, the stuffer sequence has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 58. In some aspects, the stuffer sequence has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 90. In some aspects, the stuffer sequence has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 91. SEQ ID NOs. 54-58, 90 and 91 are exemplary human factor VIII stuffer sequences that can be used in any of the vectors described herein. Additional stuffer sequences are known in the art.

### Capsids

In some aspects, the recombinant AAV vectors of the present disclosure are packaged into a capsid of the AAV2, 3, 4, 5, 6, 7, 8, 9, 10, rh8, rh10, rh39, rh43 or Anc80 serotype or one or more hybrids thereof. In some aspects, a capsid is from an ancestral serotype. For example, in some aspects, the capsid is an Anc80 capsid (e.g., an Anc80L65 capsid).

In some aspects, the capsid comprises a polypeptide with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the polypeptide of SEQ ID NO: 109. In some aspects, the capsid comprises a polypeptide represented by SEQ ID NO: 109.

Any combination of ITRs and capsids may be used in recombinant AAV vectors of the present disclosure, for example, wild-type or variant AAV2 ITRs and Anc80 capsid, wild-type or variant AAV2 ITRs and AAV6 capsid, etc. In some aspects of the present disclosure an rAAV particle is an rAAV2/Anc80 particle which comprises an Anc80 capsid (e.g., comprising a polypeptide of SEQ ID NO: 109) that encapsidates a nucleic acid vector with wild-type AAV2 ITRs (e.g., SEQ ID NOs: 97 and 104) flanking a portion of a transgene and/or construct comprising a portion of OTOF (e.g., SEQ ID NO: 101 or 107).

In some aspects, the first and second rAAV vectors are encapsulated by an AAV capsid. In some aspects, the AAV capsid encapsulating the first rAAV vector is a serotype selected from any one of AAV1, AAV2, AAV3, AAV4, AAV5, AAV6, AAV7, AAV8, AAV9, AAVrh8, AAVrh10, AAVrh39, AAVrh43, or Anc80. In some aspects, the AAV capsid encapsulating the second rAAV vector is a serotype selected from any one of AAV1, AAV2, AAV3, AAV4, AAV5, AAV6, AAV7, AAV8, AAV9, AAVrh8, AAVrh10, AAVrh39, AAVrh43, or Anc80. In some aspects, the first rAAV vector is encapsulated by an Anc80 capsid and the second rAAV vector is encapsulated by an Anc80 capsid.

In some aspects, the first and second rAAV vector genomes are encapsulated by an AAV capsid. In some aspects, the AAV capsid encapsulating the first rAAV vector genome is a serotype selected from any one of AAV1, AAV2, AAV3, AAV4, AAV5, AAV6, AAV7, AAV8, AAV9, AAVrh8, AAVrh10, AAVrh39, AAVrh43, or Anc80. In some aspects, the AAV capsid encapsulating the second rAAV vector genome is a serotype selected from any one of AAV1, AAV2, AAV3, AAV4, AAV5, AAV6, AAV7, AAV8, AAV9, AAVrh8, AAVrh10, AAVrh39, AAVrh43, or Anc80. In some aspects, the first rAAV vector genome is encapsulated by an Anc80 capsid and the second rAAV vector genome is encapsulated by an Anc80 capsid.

In some aspects, the Anc80 capsid comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 109. In some aspects, the Anc80 capsid comprises a polypeptide of SEQ ID NO: 109.

In some aspects, the first recombinant AAV particle comprises a nucleic acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 96. In some aspects, the first recombinant AAV particle comprises a nucleic acid sequence having the sequence of SEQ ID NO: 96.

In some aspects, the second recombinant AAV particle comprises a nucleic acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 105. In some aspects, the second recombinant AAV particle comprises a nucleic acid sequence having the sequence of SEQ ID NO: 105.

### Vectors

Provided herein are compositions of matter and methods of use for the treatment of hearing loss, using nucleic acid therapeutics such as auditory polypeptide messenger RNAs (e.g., otoferlin gene). In some aspects, the auditory polypeptide nucleic acids (e.g., otoferlin gene) are present in viral vectors, such as adeno-associated viral vectors, adenoviral vectors, lentiviral vectors, and retroviral vectors. In some aspects, the vectors are adeno-associated viral vectors. In some aspects, the vectors are recombinant adeno-associated viral vectors.

The foregoing methods for packaging recombinant vectors in desired AAV capsids to produce the rAAVs (or rAAV particles) of the disclosure are not meant to be limiting and other suitable methods will be apparent to the skilled artisan.

### Recombinant AAV Vectors

In some aspects, the viral vector is an adeno-associated virus (AAV) vector. In some aspects, the viral vector is a recombinant adeno-associated virus (rAAV) vector. “Recombinant AAV vectors” or “rAAVs” of the disclosure comprise at a minimum, a transgene or a portion thereof and a regulatory sequence (e.g., a promoter), and 5′ and 3′ AAV inverted terminal repeats (ITRs). It is this recombinant AAV vector which is packaged into a capsid protein and delivered to a selected target cell. In some aspects, the transgene is a nucleic acid sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

Such a recombinant AAV vector is packaged into a capsid to form an rAAV particle and delivered to a selected target cell (e.g., an inner hair cell). In some aspects, one or more recombinant AAV vectors of the present disclosure is packaged into a capsid of the AAV2, 3, 4, 5, 6, 7, 8, 9, 10, rh8, rh10, rh39, rh43 or Anc80 serotype or one or more hybrids thereof.

In some aspects, an rAAV of the disclosure is encapsidated by an Anc80 capsid. In some aspects, the Anc80 capsid comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 109. In some aspects, the Anc80 capsid comprises a polypeptide of SEQ ID NO: 109.

In some aspects of any of the compositions described herein, the first vector includes an ITR (e.g., any of the exemplary ITR sequences described herein), a promoter and/or enhancer (e.g., any of the exemplary enhancers and any of the exemplary promoters described herein), a sequence encoding a first, N-terminal portion of a human otoferlin protein (e.g., any of the exemplary sequences encoding a first, N-terminal portion of a human otoferlin protein described herein), a splicing donor site (e.g., any of the exemplary splicing donor sites described herein), an AK sequence (e.g., any of the exemplary AK sequences described herein), and an ITR (e.g., any of the exemplary ITR sequences described herein).

In some aspects of any of the compositions described herein, the second vector includes an ITR sequence (e.g., any of the exemplary ITR sequences described herein), an AK sequence (e.g., any of the exemplary AK sequences described herein), a splicing acceptor sequence (e.g., any of the splicing acceptor sequences described herein), a sequence encoding a second portion of a human otoferlin protein (e.g., any of the exemplary sequences encoding a second, C-terminal portion of a human otoferlin protein described herein), a poly(A) signal sequence (e.g., any of the exemplary poly(A) signal sequences described herein), and an ITR sequence (e.g., any of the exemplary ITR sequences described herein).

The vectors provided herein can be of different sizes. The choice of vector that is used in any of the compositions, kits, and methods described herein may depend on the size of the vector. In some aspects, the vector(s) is an adeno-associated virus (AAV vector) and can include a total number of nucleotides of up to 5 kb. In some aspects, the AAV vector(s) can include a total number of nucleotides in the range of about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

In some aspects of any of the compositions, kits, and methods provided herein, the two different vectors can be substantially the same type of vector and may differ in size. In some aspects, the two different vectors can be different types of vector, and may have substantially the same size or have different sizes.

A variety of different methods known in the art can be used to introduce any of the vectors disclosed herein into a mammalian cell (e.g., a cochlear inner hair cell). Non-limiting examples of methods for introducing nucleic acid into a mammalian cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection.

Skilled practitioners will appreciate that any of the vectors described herein can be introduced into a mammalian cell by, for example, lipofection.

Various molecular biology techniques that can be used to introduce a mutation(s) and/or a deletion(s) into an endogenous gene are also known in the art. Non-limiting examples of such techniques include site-directed mutagenesis, CRISPR (e.g., CRISPR/Cas9-induced knock-in mutations and CRISPR/Cas9-induced knock-out mutations), and TALENs. These methods can be used to correct the sequence of a defective endogenous gene present in a chromosome of a target cell.

In some aspects, a composition of the present disclosure comprises a first rAAV vector comprising a first expression cassette comprising a 5′ inverted terminal repeat, a promoter operably linked to a 5′ portion of the OTOF gene or OTOF cDNA, a splice donor sequence, a AK recombinogenic sequence, and a 3′ inverted terminal repeat.

In some aspects, a composition of the present disclosure comprises a second rAAV vector comprising a second expression cassette comprising a 5′ inverted terminal repeat, a AK recombinogenic sequence, a splice acceptor sequence, a 3′ portion of the OTOF gene or OTOF cDNA, a polyadenylation sequence, and a 3′ inverted terminal repeat.

In some aspects, a composition of the present disclosure comprises a first rAAV vector genome comprising a first expression cassette comprising a 5′ inverted terminal repeat, a promoter operably linked to a 5′ portion of the OTOF gene or OTOF cDNA, a splice donor sequence, a AK recombinogenic sequence, and a 3′ inverted terminal repeat.

In some aspects, a composition of the present disclosure comprises a second rAAV vector genome comprising a second expression cassette comprising a 5′ inverted terminal repeat, a AK recombinogenic sequence, a splice acceptor sequence, a 3′ portion of the OTOF gene or OTOF cDNA, a polyadenylation sequence, and a 3′ inverted terminal repeat.

In some aspects, a composition of the present disclosure comprises a first rAAV vector comprising a first expression cassette comprising, in 5′ to 3′ orientation, a 5′ inverted terminal repeat, a promoter operably linked to a 5′ portion of the OTOF gene or OTOF cDNA, a splice donor sequence, a AK recombinogenic sequence, and a 3′ inverted terminal repeat.

In some aspects, a composition of the present disclosure comprises a second rAAV vector comprising a second expression cassette comprising, in 5′ to 3′ orientation, a 5′ inverted terminal repeat, a AK recombinogenic sequence, a splice acceptor sequence, a 3′ portion of the OTOF gene or OTOF cDNA, a polyadenylation sequence, and a 3′ inverted terminal repeat.

In some aspects, a composition of the present disclosure comprises a first rAAV vector genome comprising a first expression cassette comprising, in 5′ to 3′ orientation, a 5′ inverted terminal repeat, a promoter operably linked to a 5′ portion of the OTOF gene or OTOF cDNA, a splice donor sequence, a AK recombinogenic sequence, and a 3′ inverted terminal repeat.

In some aspects, a composition of the present disclosure comprises a second rAAV vector genome comprising a second expression cassette comprising, in 5′ to 3′ orientation, a 5′ inverted terminal repeat, a AK recombinogenic sequence, a splice acceptor sequence, a 3′ portion of the OTOF gene or OTOF cDNA, a polyadenylation sequence, and a 3′ inverted terminal repeat.

In some aspects, a composition of the present disclosure comprises a first rAAV vector comprising a first expression cassette comprising i) a 5′ inverted terminal repeat having the sequence of SEQ ID NO: 97, ii) a promoter having the sequence of SEQ ID NOs: 98, 99, and 100 operably linked to iii) a 5′ portion of the OTOF gene or OTOF cDNA having the sequence of SEQ ID NO: 101, iv) a splice donor sequence having the sequence of SEQ ID NO: 102, v) a AK recombinogenic sequence having the sequence of SEQ ID NO: 103, and vi) a 3′ inverted terminal repeat having the sequence of SEQ ID NO: 104.

In some aspects, a composition of the present disclosure comprises a second rAAV vector comprising a second expression cassette comprising i) a 5′ inverted terminal repeat having the sequence of SEQ ID NO: 97, ii) a AK recombinogenic sequence having the sequence of SEQ ID NO: 103, iii) a splice acceptor sequence having the sequence of SEQ ID NO: 106, iv) a 3′ portion of the OTOF gene or OTOF cDNA having the sequence of SEQ ID NO: 107, v) a polyadenylation sequence having the sequence of SEQ ID NO: 108, and vi) a 3′ inverted terminal repeat having the sequence of SEQ ID NO: 104.

In some aspects, a composition of the present disclosure comprises a first rAAV vector genome comprising a first expression cassette comprising i) a 5′ inverted terminal repeat having the sequence of SEQ ID NO: 97, ii) a promoter having the sequence of SEQ ID NOs: 98, 99, and 100 operably linked to iii) a 5′ portion of the OTOF gene or OTOF cDNA having the sequence of SEQ ID NO: 101, iv) a splice donor sequence having the sequence of SEQ ID NO: 102, v) a AK recombinogenic sequence having the sequence of SEQ ID NO: 103, and vi) a 3′ inverted terminal repeat having the sequence of SEQ ID NO: 104.

In some aspects, a composition of the present disclosure comprises a second rAAV vector genome comprising a second expression cassette comprising i) a 5′ inverted terminal repeat having the sequence of SEQ ID NO: 97, ii) a AK recombinogenic sequence having the sequence of SEQ ID NO: 103, iii) a splice acceptor sequence having the sequence of SEQ ID NO: 106, iv) a 3′ portion of the OTOF gene or OTOF cDNA having the sequence of SEQ ID NO: 107, v) a polyadenylation sequence having the sequence of SEQ ID NO: 108, and vi) a 3′ inverted terminal repeat having the sequence of SEQ ID NO: 104.

In some aspects, a composition of the present disclosure comprises a first rAAV vector comprising a first expression cassette comprising, in 5′ to 3′ orientation, i) a 5′ inverted terminal repeat having the sequence of SEQ ID NO: 97, ii) a promoter having the sequence of SEQ ID NOs: 98, 99, and 100 operably linked to iii) a 5′ portion of the OTOF gene or OTOF cDNA having the sequence of SEQ ID NO: 101, iv) a splice donor sequence having the sequence of SEQ ID NO: 102, v) a AK recombinogenic sequence having the sequence of SEQ ID NO: 103, and vi) a 3′ inverted terminal repeat having the sequence of SEQ ID NO: 104.

In some aspects, a composition of the present disclosure comprises a second rAAV vector comprising a second expression cassette comprising, in 5′ to 3′ orientation, i) a 5′ inverted terminal repeat having the sequence of SEQ ID NO: 97, ii) a AK recombinogenic sequence having the sequence of SEQ ID NO: 103, iii) a splice acceptor sequence having the sequence of SEQ ID NO: 106, iv) a 3′ portion of the OTOF gene or OTOF cDNA having the sequence of SEQ ID NO: 107, v) a polyadenylation sequence having the sequence of SEQ ID NO: 108, and vi) a 3′ inverted terminal repeat having the sequence of SEQ ID NO: 104.

In some aspects, a composition of the present disclosure comprises a first rAAV vector genome comprising a first expression cassette comprising, in 5′ to 3′ orientation, i) a 5′ inverted terminal repeat having the sequence of SEQ ID NO: 97, ii) a promoter having the sequence of SEQ ID NOs: 98, 99, and 100 operably linked to iii) a 5′ portion of the OTOF gene or OTOF cDNA having the sequence of SEQ ID NO: 101, iv) a splice donor sequence having the sequence of SEQ ID NO: 102, v) a AK recombinogenic sequence having the sequence of SEQ ID NO: 103, and vi) a 3′ inverted terminal repeat having the sequence of SEQ ID NO: 104.

In some aspects, a composition of the present disclosure comprises a second rAAV vector genome comprising a second expression cassette comprising, in 5′ to 3′ orientation, i) a 5′ inverted terminal repeat having the sequence of SEQ ID NO: 97, ii) a AK recombinogenic sequence having the sequence of SEQ ID NO: 103, iii) a splice acceptor sequence having the sequence of SEQ ID NO: 106, iv) a 3′ portion of the OTOF gene or OTOF cDNA having the sequence of SEQ ID NO: 107, v) a polyadenylation sequence having the sequence of SEQ ID NO: 108, and vi) a 3′ inverted terminal repeat having the sequence of SEQ ID NO: 104.

In some aspects, a composition of the present disclosure comprises a first rAAV vector comprising a first expression cassette the sequence of SEQ ID NO: 96. In some aspects, a composition of the present disclosure comprises a second rAAV vector comprising a second expression cassette comprising the sequence of SEQ ID NO: 105.

In some aspects, a composition of the present disclosure comprises a first rAAV vector genome comprising a first expression cassette the sequence of SEQ ID NO: 96. In some aspects, a composition of the present disclosure comprises a second rAAV vector genome comprising a second expression cassette comprising the sequence of SEQ ID NO: 105.

In some aspects, the disclosure provides a composition comprising a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition comprises about 4.1E10-8.1E12 total vg.

In some aspects, the disclosure provides a composition comprising a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition comprises about 4.1E10-4.1E12 total vg or about 8.1E10-8.1E12 total vg.

In some aspects, the composition comprises the first rAAV vector genome and the second rAAV vector genome in about a 1:1 ratio.

In some aspects, the composition comprises about 4.1E10-8.1E12 total vg. In some aspects, the composition comprises about 4.1E10-4.1E12 total vg or about 8.1E10-8.1E12 total vg.

In some aspects, the composition comprises about 4.1E10-4.1E12, about 5.1E10-3.1E12, about 6.1E10-2.1E12, about 7.1E10-1.1E12, about 8.1E10-10.1E11, about 9.1E10-9.1E11, about 10.1E11-8.1E11, about 1.1E11-7.1E11, about 2.1E11-6.1E11, or about 3.1E11-5.1E11 total vg. In some aspects, the composition comprises about 3.1E11-5.1E11, about 3.2E11-5.0E11, about 3.3E11-4.9E11, about 3.4E11-4.8E11, about 3.5E11-4.7E11, about 3.6E11-4.6E11, about 3.7E11-4.5E11, about 3.8E11-4.4E11, about 3.9E11-4.3E11, or about 4.0E11-4.2E11 total vg. In some aspects, the composition comprises about 4.1E11 total vg.

In some aspects, the composition comprises about 4.1E10-8.1E12 total vg/cochlea. In some aspects, the composition comprises about 4.1E10-4.1E12 total vg/cochlea or about 8.1E10-8.1E12 total vg/cochlea.

In some aspects, the composition comprises about 4.1E10-4.1E12, about 5.1E10-3.1E12, about 6.1E10-2.1E12, about 7.1E10-1.1E12, about 8.1E10-10.1E11, about 9.1E10-9.1E11, about 10.1E11-8.1E11, about 1.1E11-7.1E11, about 2.1E11-6.1E11, or about 3.1E11-5.1E11 total vg/cochlea. In some aspects, the composition comprises about 3.1E11-5.1E11, about 3.2E11-5.0E11, about 3.3E11-4.9E11, about 3.4E11-4.8E11, about 3.5E11-4.7E11, about 3.6E11-4.6E11, about 3.7E11-4.5E11, about 3.8E11-4.4E11, about 3.9E11-4.3E11, or about 4.0E11-4.2E11 total vg/cochlea. In some aspects, the composition comprises about 4.1E11 total vg/cochlea.

In some aspects, the composition comprises about 3.0E11, about 3.1E11, about 3.2E11, about 3.3E11, about 3.4E11, about 3.5E11, about 3.6E11, about 3.7E11, about 3.8E11, about 3.9E11, about 4.0E11, about 4.1E11, about 4.2E11, about 4.3E11, about 4.4E11, about 4.5E11, about 4.6E11, about 4.7E11, about 4.8E11, about 4.9E11, about 5.0E11, about 5.1E11, about 5.2E11, about 5.3E11, about 5.4E11, about 5.5E11, about 5.6E11, about 5.7E11, about 5.8E11, about 5.9E11, about 6.0E11, about 6.1E11, about 6.2E11, about 6.3E11, about 6.4E11, about 6.5E11, about 6.6E11, about 6.7E11, about 6.8E11, about 6.9E11, about 7.0E11, about 7.1E11, about 7.2E11, about 7.3E11, about 7.4E11, about 7.5E11, about 7.6E11, about 7.7E11, about 7.8E11, about 7.9E11, about 8.0E11, about 8.1E11, about 8.2E11, about 8.3E11, about 8.4E11, about 8.5E11, about 8.6E11, about 8.7E11, about 8.8E11, or about 8.9E11 total vg.

In some aspects, the composition comprises about 3.0E11, about 3.1E11, about 3.2E11, about 3.3E11, about 3.4E11, about 3.5E11, about 3.6E11, about 3.7E11, about 3.8E11, about 3.9E11, about 4.0E11, about 4.1E11, about 4.2E11, about 4.3E11, about 4.4E11, about 4.5E11, about 4.6E11, about 4.7E11, about 4.8E11, about 4.9E11, about 5.0E11, about 5.1E11, about 5.2E11, about 5.3E11, about 5.4E11, about 5.5E11, about 5.6E11, about 5.7E11, about 5.8E11, about 5.9E11, about 6.0E11, about 6.1E11, about 6.2E11, about 6.3E11, about 6.4E11, about 6.5E11, about 6.6E11, about 6.7E11, about 6.8E11, about 6.9E11, about 7.0E11, about 7.1E11, about 7.2E11, about 7.3E11, about 7.4E11, about 7.5E11, about 7.6E11, about 7.7E11, about 7.8E11, about 7.9E11, about 8.0E11, about 8.1E11, about 8.2E11, about 8.3E11, about 8.4E11, about 8.5E11, about 8.6E11, about 8.7E11, about 8.8E11, or about 8.9E11 total vg/cochlea.

In some aspects, the composition comprises about 8.1E10-8.1E12, about 9.1E10-7.1E12, about 10.1E10-6.1E12, about 1.1E11-5.1E12, about 2.1E11-4.1E12, about 3.1E11-3.1E12, about 4.1E11-2.1E12, about 5.1E11-1.1E12, about 6.1E11-10.1E11, or about 7.1E11-9.1E11 total vg. In some aspects, the composition comprises about 7.1E11-9.1E11, about 7.2E11-9.0E11, about 7.3E11-8.9E11, about 7.4E11-8.8E11, about 7.5E11-8.7E11, about 7.6E11-8.6E11, about 7.7E11-8.5E11, about 7.8E11-8.4E11, about 7.9E11-8.3E11, or about 8.0E11-8.2E11 total vg. In some aspects, the composition comprises about 8.1E11 total vg.

In some aspects, the composition comprises about 8.1E10-8.1E12, about 9.1E10-7.1E12, about 10.1E10-6.1E12, about 1.1E11-5.1E12, about 2.1E11-4.1E12, about 3.1E11-3.1E12, about 4.1E11-2.1E12, about 5.1E11-1.1E12, about 6.1E11-10.1E11, or about 7.1E11-9.1E11 total vg/cochlea. In some aspects, the composition comprises about 7.1E11-9.1E11, about 7.2E11-9.0E11, about 7.3E11-8.9E11, about 7.4E11-8.8E11, about 7.5E11-8.7E11, about 7.6E11-8.6E11, about 7.7E11-8.5E11, about 7.8E11-8.4E11, about 7.9E11-8.3E11, or about 8.0E11-8.2E11 total vg/cochlea. In some aspects, the composition comprises about 8.1E11 total vg/cochlea.

In some aspects, the concentration of the composition comprises about 4.5E11-9E13 total vg/mL. In some aspects, the concentration of the composition comprises about 4.5E11-4.5E13 total vg/mL or about 9E11-9E13 total vg/mL.

In some aspects, the concentration of the composition comprises about 4.5E11-4.5E13 total vg/mL. In some aspects, the concentration of the composition comprises about 5.5E11-3.5E13, about 6.5E11-2.5E13, about 7.5E11-1.5E13, about 8.5E11-10.5E12, about 9.5E11-9.5E12, about 10.5E11-8.5E12, about 1.5E12-7.5E12, about 2.5E12-6.5E12, or about 3.5E12-5.5E12 total vg/mL. In some aspects, the concentration of the composition comprises about 3.5E12-5.5E12, about 3.6E12-5.4E12, about 3.7E12-5.3E12, about 3.8E12-5.2E12, about 3.9E12-5.1E12, about 4.0E12-5.0E12, about 4.1E12-4.9E12, about 4.2E12-4.8E12, about 4.3E12-4.7E12, or about 4.4E12-4.6E12 total vg/mL. In some aspects, the concentration of the composition comprises about 4.5E12 total vg/mL.

In some aspects, the concentration of the composition comprises about 3.5E12, about 3.6E12, about 3.7E12, about 3.8E12, about 3.9E12, about 4.0E12, about 4.1E12, about 4.2E12, about 4.3E12, about 4.4E12, about 4.5E12, about 4.6E12, about 4.7E12, about 4.8E12, about 4.9E12, about 5.0E12, about 5.1E12, about 5.2E12, about 5.3E12, about 5.4E12, about 5.5E12, about 5.6E12, about 5.7E12, about 5.8E12, about 5.9E12, about 6.0E12, about 6.1E12, about 6.2E12, about 6.3E12, about 6.4E12, about 6.5E12, about 6.6E12, about 6.7E12, about 6.8E12, about 6.9E12, about 7.0E12, about 7.1E12, about 7.2E12, about 7.3E12, about 7.4E12, about 7.5E12, about 7.6E12, about 7.7E12, about 7.8E12, about 7.9E12, about 8.0E12, about 8.1E12, about 8.2E12, about 8.3E12, about 8.4E12, about 8.5E12, about 8.6E12, about 8.7E12, about 8.8E12, about 8.9E12, about 9.0E12, about 9.1E12, about 9.2E12, about 9.3E12, about 9.4E12, about 9.5E12, about 9.6E12, about 9.7E12, about 9.8E12, or about 9.9E12 total vg/mL.

In some aspects, the concentration of the composition comprises about 9E11-9E13 total vg/mL. In some aspects, the concentration of the composition comprises about 9E11-9E13, about 10E11-8E13, about 1E12-7E13, about 2E12-6E13, about 3E12-5E13, about 4E12-4E13, about 5E12-3E13, about 6E12-2E13, about 7E12-1E13, or about 8E12-10E12 total vg/mL. In some aspects, the concentration of the composition comprises about 8E12-10E12, about 8.1E12-9.9E12, about 8.2E12-9.8E12, about 8.3E12-9.7E12, about 8.4E12-9.6E12, about 8.5E12-9.5E12, about 8.6E12-9.4E12, about 8.7E12-9.3E12, about 8.8E12-9.2E12, or about 8.9E12-9.1E12. In some aspects, the concentration of the composition comprises 9E12 total vg/mL.

In some aspects, the first and second rAAV vectors are capable of constituting a polypeptide messenger RNA encoding a full length human otoferlin protein in the subject. In some aspects, the first and second rAAV vectors are capable of reconstituting an active otoferlin gene (e.g., a full-length otoferlin gene) within a cell following intermolecular i) concatamerization, ii) recombination, iii) trans-splicing, iv) concatamerization and trans-splicing, or v) recombination and trans-splicing.

### Mammalian Cells

Also provided herein is a cell (e.g., a mammalian cell) that includes any of the nucleic acids, vectors (e.g., at least two different vectors described herein), or compositions described herein. Skilled practitioners will appreciate that the nucleic acids and vectors described herein can be introduced into any mammalian cell. Non-limiting examples of vectors and methods for introducing vectors into mammalian cells are described herein. In some aspects, the cell is a human cell, a mouse cell, a porcine cell, a rabbit cell, a dog cell, a cat cell, a rat cell, a sheep cell, a cat cell, a horse cell, or a non-human primate cell. In some aspects, the cell is a specialized cell of the cochlea. In some aspects, the cell is a cochlear inner hair cell or a cochlear outer hair cell. In some aspects, the cell is a cochlear inner hair cell. In some aspects, the cell is a cochlear inner hair cell.

In some aspects, the mammalian cell is in vitro. In some aspects, the mammalian cell is present in a mammal. In some aspects, the mammalian cell is a human cell. In some aspects, the mammalian cell is obtained from a subject. In some aspects, the mammalian cell is an autologous cell obtained from a subject and/or is cultured ex vivo.

### Methods of Use

Method of Introduction into Cochlea

Also provided herein is a method of introducing into a cochlea of a mammal (e.g., a human) the compositions described herein.

Also provided are methods of increasing expression of an active otoferlin protein (e.g., a full-length otoferlin protein) in an inner hair cell in a cochlea of a mammal (e.g., a human) that include introducing into the cochlea of the any of the compositions described herein.

Also provided are methods of treating hearing loss in a subject (e.g., a human) identified as having a defective otoferlin gene (e.g., an otoferlin gene having a mutation that results in a decrease in the expression and/or activity of an otoferlin protein encoded by the gene), wherein the methods include administering any of the compositions described herein into a cochlea of a subject.

Also provided are methods of treating non-syndromic sensorineural hearing loss in a subject (e.g., a human) identified as having a defective otoferlin gene (e.g., an otoferlin gene having a mutation that results in a decrease in the expression and/or activity of an otoferlin protein encoded by the gene), wherein the methods include administering any of the compositions described herein into a cochlea of a subject.

Also provided are methods of treating hearing loss in a subject (e.g., a human) identified as having biallelic otoferlin gene mutations, wherein the methods include administering any of the compositions described herein into a cochlea of a subject.

In some aspects, the methods described herein can further include administering a neurotrophic factor to a cochlea of a subject (e.g., at substantially the same time as or before, or after, any of the compositions described herein are administered to the subject). In some examples, the methods described herein can further include administering a cochlear implant to a subject (e.g., at substantially the same time as or before, or after, any of the compositions described herein are administered to the subject).

In some aspects of any of these methods, the mammal has been previously identified as having a defective otoferlin gene (e.g., an otoferlin gene having a mutation that results in a decrease in the expression and/or activity of an otoferlin protein encoded by the gene). In some aspects, these methods further include, prior to the introducing or administering step, determining that the subject has a defective otoferlin gene. In some aspects, these methods can further include detecting a mutation in an otoferlin gene in a subject. In some aspects, the methods can further include identifying or diagnosing a subject as having non-syndromic sensorineural hearing loss.

In some aspects, the subject identified as having biallelic otoferlin gene mutations has been previously identified as having biallelic otoferlin gene mutations. In some aspects, these methods further include, prior to the introducing or administering step, determining that the subject has biallelic otoferlin gene mutations. In some aspects, the biallelic otoferlin gene mutations can comprise any of the mutations of an otoferlin gene described herein. In some aspects, the biallelic otoferlin gene mutations cause localized synaptic transmission defects between hair cells and the auditory nerve in the subject.

In some aspects, the mammal or the subject has a clinical presentation of profound sensorineural hearing loss. In some aspects, the profound sensorineural hearing loss is bilateral profound sensorineural hearing loss. In some aspects, the subject identified as having a defective otoferlin gene has the clinical presentation of profound sensorineural hearing loss when afebrile.

In some aspects, the mammal or the subject has preserved distortion product otoacoustic emissions (DPOAEs). In some aspects, outer hair cells of the mammal or the subject function normally.

In some aspects, the methods can include administration of a single dose of the compositions described herein to the cochlea of the mammal or the subject.

In some aspects, these methods can include introducing or administering a first dose of the composition into the cochlea of the mammal or subject, assessing hearing function of the mammal or subject following the introducing or the administering of the first dose, and administering an additional dose of the composition into the cochlea of the mammal or subject found not to have a hearing function within a normal range (e.g., as determined using any test for hearing known in the art).

In some aspects, the composition is administered in a plurality of doses. In some aspects, the composition is administered in 2, 3, 4, 5, 6, 7, 8, 9, or 10 doses.

In some aspects, a single dose comprises about 0.01 mL-0.2 mL. In some aspects, a single dose comprises about 0.01-0.2, about 0.02-0.18, about 0.03-0.16, about 0.04-0.14, about 0.05-0.13, about 0.06-0.12, about 0.07-0.11, about 0.08-0.10 mL. In some aspects, a single dose comprises about 0.01, about 0.02, about 0.03, about 0.04, about 0.05, about 0.06, about 0.07, about 0.08, about 0.09, about 0.1, about 0.11, about 0.012, about 0.13, about 0.14, about 0.15, about 0.16, about 0.17, about 0.18, about 0.19, or about 0.2 mL. In some aspects, a single dose comprises about 0.09 mL.

In some aspects, the composition is administered in a single injection. In some aspects, the composition is administered in a plurality of injections. In some aspects, the composition is administered in 2, 3, 4, 5, 6, 7, 8, 9, or 10 injections.

In some aspects of any of the methods described herein, the composition can be formulated for intra-cochlear administration. In some aspects, the composition comprises one or more pharmaceutically acceptable carriers, diluents, or excipients. In some aspects, the composition further comprises one or more buffering agents and one or more surfactants. In some aspects, the buffering agents are selected from monopotassium phosphate, sodium phosphate dibasic, potassium chloride, sodium chloride, Tris HCl, Tris base, histidine, boric acid, citric acid, glycine, HEPES, and MOPS. In some aspects, the surfactant is selected from poloxamer 188, labrasol, tween, ethanol, pluronic F68, and polyethyelene glycol. In some aspects, the composition comprises monopotassium phosphate, sodium phosphate dibasic, potassium chloride, sodium chloride, and Poloxamer 188.

In some aspects, the composition comprises a) about 1.35-1.65 mM monopotassium phosphate; b) about 7.29-8.91 mM sodium phosphate dibasic; c) about 2.43-2.97 mM potassium chloride; d) about 154.8-189.2 mM sodium chloride; and e) about 0.0001%-0.01% Poloxamer 188.

In some aspects, the composition comprises a) about 1.35, about 1.375, about 1.4, about 1.425, about 1.45, about 1.475, about 1.5, about 1.525, about 1.55, about 1.575, about 1.6, about 1.625, or about 1.65 mM monopotassium phosphate; b) about 7.29, about 7.4, about 7.5 about 7.6, about 7.7 about 7.8, about 7.9, about 8.0, about 8.1, about 8.2, about 8.3, about 8.4, about 8.5, about 8.6, about 8.7, about 8.8, or about 8.91 mM sodium phosphate dibasic; c) about 2.43, about 2.5, about 2.55, about 2.6, about 2.65, about 2.7, about 2.75, about 2.8, about 2.85, about 2.9, about 2.95, or about 2.97 mM potassium chloride; d) about 154.8, about 160, about 165, about 170, about 175, about 180, about 185, or about 189.2 mM sodium chloride, and e) about 0.0001%, about 0.00025% about 0.0005%, about 0.00075%, about 0.001%, about 0.0025%, about 0.005%, about 0.0075%, or about 0.01% poloxamer 188.

In some aspects, the composition comprises: a) about 1.5 mM monopotassium phosphate; b) about 8.1 mM sodium phosphate dibasic; c) about 2.7 mM potassium chloride; d) about 172 mM sodium chloride; and e) about 0.001% Poloxamer 188.

In some aspects, the composition is formulated to comprise a synthetic perilymph solution. In some aspects, the synthetic perilymph solution comprises one or more pharmaceutically acceptable carriers, diluents, or excipients. In some aspects, the synthetic perilymph solution further comprises one or more buffering agents and one or more surfactants. In some aspects, the buffering agents are selected from monopotassium phosphate, sodium phosphate dibasic, potassium chloride, sodium chloride, Tris HCl, Tris base, histidine, boric acid, citric acid, glycine, HEPES, and MOPS. In some aspects, the surfactant is selected from poloxamer 188, labrasol, tween, ethanol, pluronic F68, and polyethyelene glycol. In some aspects, the synthetic perilymph solution comprises monopotassium phosphate, sodium phosphate dibasic, potassium chloride, sodium chloride, and Poloxamer 188.

In some aspects, the synthetic perilymph solution comprises a) about 1.35-1.65 mM monopotassium phosphate; b) about 7.29-8.91 mM sodium phosphate dibasic; c) about 2.43-2.97 mM potassium chloride; d) about 154.8-189.2 mM sodium chloride; and e) about 0.0001%-0.01% Poloxamer 188.

In some aspects, the synthetic perilymph solution comprises a) about 1.35, about 1.375, about 1.4, about 1.425, about 1.45, about 1.475, about 1.5, about 1.525, about 1.55, about 1.575, about 1.6, about 1.625, or about 1.65 mM monopotassium phosphate; b) about 7.29, about 7.4, about 7.5 about 7.6, about 7.7 about 7.8, about 7.9, about 8.0, about 8.1, about 8.2, about 8.3, about 8.4, about 8.5, about 8.6, about 8.7, about 8.8, or about 8.91 mM sodium phosphate dibasic; c) about 2.43, about 2.5, about 2.55, about 2.6, about 2.65, about 2.7, about 2.75, about 2.8, about 2.85, about 2.9, about 2.95, or about 2.97 mM potassium chloride; d) about 154.8, about 160, about 165, about 170, about 175, about 180, about 185, or about 189.2 mM sodium chloride, and e) about 0.0001%, about 0.00025% about 0.0005%, about 0.00075%, about 0.001%, about 0.0025%, about 0.005%, about 0.0075%, or about 0.01% poloxamer 188.

In some aspects, the synthetic perilymph solution comprises: a) about 1.5 mM monopotassium phosphate; b) about 8.1 mM sodium phosphate dibasic; c) about 2.7 mM potassium chloride; d) about 172 mM sodium chloride; and e) about 0.001% Poloxamer 188.

In some aspects, the compositions described herein can be administered via intra-cochlear administration or local administration. In some aspects, the compositions are administered through the use of a medical device (e.g., any of the exemplary medical devices described herein). In some aspects, the composition is pre-loaded in the device.

In some aspects, the device is a device as shown in FIGS. 2-5. In some aspects, the device is a microcatheter. In some aspects, the microcatheter is shaped such that it can enter the middle ear cavity via the external auditory canal and contact the end of the microcatheter with the round window membrane (RWM). In some aspects a distal end of the microcatheter comprises at least one microneedle with a diameter of between 10 and 1,000 microns. In some aspects, the at least one microneedle comprises a bent portion and an angled tip.

In some aspects, intra-cochlear administration can be performed using any of the methods described herein or known in the art. For example, a composition can be administered or introduced into the cochlea using the following surgical technique: first using visualization with a 0 degree, 2.5-mm rigid endoscope, the external auditory canal is cleared and a round knife is used to sharply delineate an approximately 5-mm tympanomeatal flap. The tympanomeatal flap is then elevated and the middle ear is entered posteriorly. The chorda tympani nerve is identified and divided, and a currette is used to remove the scutal bone, exposing the round window membrane. To enhance apical distribution of the administered or introduced composition, a surgical laser may be used to make a small 2-mm fenestration in the oval window to allow for perilymph displacement during trans-round window membrane infusion of the composition. The microinfusion device is then primed and brought into the surgical field. The device is maneuvered to the round window, and the tip is seated within the bony round window overhang to allow for penetration of the membrane by the microneedle(s). The footpedal is engaged to allow for a measured, steady infusion of the composition. The device is then withdrawn and the round window and stapes foot plate are sealed with a gelfoam patch.

In some aspects, the present disclosure describes a delivery approach that utilizes a minimally invasive, well-accepted surgical technique for accessing the middle ear and/or inner ear through the external auditory canal. The procedure includes opening one of the physical barriers between the middle and inner ear at the oval window, and subsequently using a device disclosed herein, e.g., as shown in FIGS. 2-5 (or microcatheter) to deliver a composition disclosed herein at a controlled flow rate and in a fixed volume, via the round window membrane.

In some aspects, surgical procedures for mammals (e.g., rodents (e.g., mice, rats, hamsters, or rabbits), primates (e.g., NHP (e.g., macaque, chimpanzees, monkeys, or apes) or humans) may include venting to increase AAV vector transduction rates along the length of the cochlea. In some aspects, absence of venting during surgery may result in lower AAV vector cochlear cell transduction rates when compared to AAV vector cochlear cell transduction rates following surgeries performed with venting. In some aspects, venting facilitates transduction rates of about 75-100% of IHCs throughout the cochlea. In some aspects, venting permits IHC transduction rates of about 50-70%, about 60-80%, about 70-90%, or about 80-100% at the base of the cochlea. In some aspects, venting permits IHC transduction rates of about 50-70%, about 60-80%, about 70-90%, or about 80-100% at the apex of the cochlea.

A delivery device described herein may be placed in a sterile field of an operating room and the end of a tubing may be removed from the sterile field and connected to a syringe that has been loaded with a composition disclosed herein (e.g., one or more AAV vectors) and mounted in the pump. After appropriate priming of the system in order to remove any air, a needle may then be passed through the middle ear under visualization (surgical microscope, endoscope, and/or distal tip camera). A needle (or microneedle) may be used to puncture the RWM. The needle may be inserted until a stopper contacts the RWM. The device may then be held in that position while a composition disclosed herein is delivered at a controlled flow rate to the inner ear, for a selected duration of time. In some aspects, the flow rate (or infusion rate) may include a rate of about 30 μL/min, or from about 25 μL/min to about 35 μL/min, or from about 20 μL/min to about 40 μL/min, or from about 20 μL/min to about 70 μL/min, or from about 20 μL/min to about 90 μL/min, or from about 20 μL/min to about 100 μL/min. In some aspects, the flow rate is about 20 μL/min, about 30 μL/min, about 40 μL/min, about 50 μL/min, about 60 μL/min, about 70 μL/min, about 80 μL/min, about 90 μL/min or about 100 μL/min. In some aspects, the selected duration of time (that is, the time during which a composition disclosed herein is flowing) may be about 3 minutes, or from about 2.5 minutes to about 3.5 minutes, or from about 2 minutes to about 4 minutes, or from about 1.5 minutes to about 4.5 minutes, or from about 1 minute to about 5 minutes. In some aspects, the total volume of a composition disclosed herein that flows to the inner ear may be about 0.09 mL, or from about 0.08 mL to about 0.10 mL, or from about 0.07 mL to about 0.11 mL.

In some aspects, a single dose comprises about 0.01 mL-0.2 mL. In some aspects, a single dose comprises about 0.01-0.2, about 0.02-0.18, about 0.03-0.16, about 0.04-0.14, about 0.05-0.13, about 0.06-0.12, about 0.07-0.11, about 0.08-0.10 mL. In some aspects, a single dose comprises about 0.01, about 0.02, about 0.03, about 0.04, about 0.05, about 0.06, about 0.07, about 0.08, about 0.09, about 0.1, about 0.11, about 0.012, about 0.13, about 0.14, about 0.15, about 0.16, about 0.17, about 0.18, about 0.19, or about 0.2 mL. In some aspects, a single dose comprises about 0.09 mL.

In some aspects, the total volume of a composition disclosed herein equates to from about 40% to about 50% of the volume of the inner ear.

Once the delivery has been completed, the device may be removed. In some embodiments, a device described herein, may be configured as a single-use disposable product. In other aspects, a device described herein may be configured as a multi-use, sterilizable product, for example, with a replaceable and/or sterilizable needle sub-assembly. Single use devices may be appropriately discarded (for example, in a biohazard sharps container) after administration is complete.

In some embodiments, a composition disclosed herein comprises one or a plurality of AAV vectors. In some aspects, when more than one AAV vector is included in the composition, the AAV vectors are each different. In some aspects, an AAV vector comprises an OTOF coding region, e.g., as described herein. In some aspects, a composition comprises an rAAV particle comprising an AAV vector described herein. In some aspects, the r AAV particle is encapsidated by an Anc80 capsid. In some aspect, the Anc80 capsid comprises a polypeptide of SEQ ID NO: 109.

### Subjects

In some aspects of any of the methods described herein, the subject or mammal is a rodent, a non-human primate, or a human. In some aspects of any of the methods described herein, the subject or mammal is an adult, a teenager, a juvenile, a child, a toddler, an infant, or a newborn. In some aspects of any of the methods described herein, the subject or mammal is 1-5, 1-10, 1-20, 1-30, 1-40, 1-50, 1-60, 1-70, 1-80, 1-90, 1-100, 1-110, 2-5, 2-10, 10-20, 20-30, 30-40, 40-50, 50-60, 60-70, 70-80, 80-90, 90-100, 100-110, 10-30, 10-40, 10-50, 10-60, 10-70, 10-80, 10-90, 10-100, 10-110, 20-40, 20-50, 20-60, 20-70, 20-80, 20-90, 20-100, 20-110, 30-50, 30-60, 30-70, 30-80, 30-90, 30-100, 40-60, 40-70, 40-80, 40-90, 40-100, 50-70, 50-80, 50-90, 50-100, 60-80, 60-90, 60-100, 70-90, 70-100, 70-110, 80-100, 80-110, or 90-110 years of age. In some aspects of any of the methods described herein, the subject or mammal is 1, 2, 3, 4, 5, 6, 7, 8, 9, 10, or 11 months of age. In some aspects, the subject is between 2 and 17 years of age.

In some aspects of any of the methods described herein, the methods result in improvement in hearing (e.g., any of the metrics for determining improvement in hearing described herein) in a subject in need thereof for at least 10 days, at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 100 days, at least 105 days, at least 110 days, at least 115 days, at least 120 days, at least 5 months, at least 6 months, at least 7 months, at least 8 months, at least 9 months, at least 10 months, at least 11 months, or at least 12 months.

In some aspects, the subject or mammal has or is at risk of developing non-syndromic sensorineural hearing loss. In some aspects, the subject or mammal has been identified as having a defective otoferlin gene. In some aspects, the subject or mammal has been previously identified as having a mutation in an otoferlin gene. In some aspects, the subject or mammal has any of the mutations in an otoferlin gene that are described herein or are known in the art to be associated with non-syndromic sensorineural hearing loss.

In some aspects, the subject or mammal has been identified as being a carrier of a mutation in an otoferlin gene (e.g., via genetic testing). In some aspects, the subject or human has been identified as having a mutation in an otoferlin gene and has been diagnosed with non-syndromic sensorineural hearing loss. In some aspects, the subject or human has been identified as having non-syndromic sensorineural hearing loss.

In some aspects, successful treatment of non-syndromic sensorineural hearing loss can be determined in a subject using any of the conventional functional hearing tests known in the art. Non-limiting examples of functional hearing tests are various types of audiometric assays (e.g., pure-tone testing, speech testing, test of the middle ear, auditory brainstem response, and otoacoustic emissions). In some aspects, the administration of the composition improves the subject's or mammal's auditory brainstem response (ABR) threshold response, age-appropriate behavioral audiometry, tympanometry, and/or word/sentence recognition testing.

### Increasing Expression of Active Otoferlin

Also provided herein are methods of increasing expression of an active otoferlin protein (e.g., a full-length otoferlin protein) in a mammalian cell that include introducing any of the compositions described herein into a mammalian cell. In some aspects, a mammalian cell is a cochlear cell. In some aspects, the mammalian cell is a inner ear hair cell. In some aspects, a mammalian cell is a human cell (e.g., a human cochlear inner hair cell). In some aspects, a mammalian cell is in vitro. In some aspects, a mammalian cell is in a mammal. In some aspects, a mammalian cell is originally obtained from a mammal and/or is cultured ex vivo. In some aspects, a mammalian cell has previously been determined to have a defective otoferlin gene.

Methods for introducing any of the compositions described herein into a mammalian cell are known in the art (e.g., the use of a viral vector, e.g., any of the viral vectors described herein).

In some aspects, an increase in expression of an active otoferlin protein (e.g., a full-length otoferlin protein) as described herein is, e.g., as compared to a control or to the level of expression of an active otoferlin protein (e.g., a full-length otoferlin protein) prior to the introduction of the vector(s).

### Methods of Detecting Otoferlin

Methods of detecting expression and/or activity of otoferlin are known in the art. In some aspects, the level of expression of an otoferlin protein can be detected directly (e.g., detecting otoferlin protein or detecting otoferlin mRNA). Non-limiting examples of techniques that can be used to detect expression and/or activity of otoferlin directly include: real-time PCR, Western blotting, immunoprecipitation, immunohistochemistry, or immunofluorescence. In some aspects, expression of an otoferlin protein can be detected indirectly (e.g., through functional hearing tests).

### Dosing and Volume of Administration

In some aspects, a composition disclosed herein is administered as a single dose or as a plurality of doses. In some aspects, the composition is administered in a single dose. In some aspects, the composition is administered in a plurality of doses. In some aspects, the composition is administered in 2, 3, 4, 5, 6, 7, 8, 9, or 10 doses.

In some aspects, the composition is administered in a single injection. In some aspects, the composition is administered in a plurality of injections. In some aspects, the composition is administered in 2, 3, 4, 5, 6, 7, 8, 9, or 10 injections.

In some aspects, a composition disclosed herein (e.g., a composition comprising one or a plurality of AAV vectors disclosed herein) is administered at a volume of about 0.01 mL, about 0.02 mL, about 0.03 mL, about 0.04 mL, about 0.05 mL, about 0.06 mL, about 0.07 mL, about 0.08 mL, about 0.09 mL, about 1.00 mL, about 1.10 mL, about 1.20 mL, about 1.30 mL, about 1.40 mL, about 1.50 mL, about 1.60 mL, about 1.70 mL, about 1.80 mL, about 1.90 mL, or about 2.00 mL. In some aspects, a composition disclosed herein is administered at a volume of about 0.01 mL. In some aspects, a composition disclosed herein is administered at a volume of about 0.02 mL. In some aspects, a composition disclosed herein is administered at a volume of about 0.03 mL. In some aspects, a composition disclosed herein is administered at a volume of about 0.04 mL. In some aspects, a composition disclosed herein is administered at a volume of about 0.05 mL. In some aspects, a composition disclosed herein is administered at a volume of about 0.06 mL. In some aspects, a composition disclosed herein is administered at a volume of about 0.07 mL. In some aspects, a composition disclosed herein is administered at a volume of about 0.08 mL. In some aspects, a composition disclosed herein is administered at a volume of about 0.09 mL. In some aspects, a composition disclosed herein is administered at a volume of about 1.00 mL. In some aspects, a composition disclosed herein is administered at a volume of about 1.10 mL. In some aspects, a composition disclosed herein is administered at a volume of about 1.20 mL. In some aspects, a composition disclosed herein is administered at a volume of about 1.30 mL. In some aspects, a composition disclosed herein is administered at a volume of about 1.40 mL. In some aspects, a composition disclosed herein is administered at a volume of about 1.50 mL. In some aspects, a composition disclosed herein is administered at a volume of about 1.60 mL. In some aspects, a composition disclosed herein is administered at a volume of about 1.70 mL. In some aspects, a composition disclosed herein is administered at a volume of about 1.80 mL. In some aspects, a composition disclosed herein is administered at a volume of about 1.90 mL. In some aspects, a composition disclosed herein is administered at a volume of about 2.00 mL.

In some aspects, a composition disclosed herein (e.g., a composition comprising one or a plurality of AAV vectors disclosed herein) is administered at a volume of about 0.01 to 2.00 mL, about 0.02 to 1.90 mL, about 0.03 to 1.8 mL, about 0.04 to 1.70 mL, about 0.05 to 1.60 mL, about 0.06 to 1.50 mL, about 0.06 to 1.40 mL, about 0.07 to 1.30 mL, about 0.08 to 1.20 mL, or about 0.09 to 1.10 mL. In some aspects a composition disclosed herein (e.g., a composition comprising one or a plurality of AAV vectors disclosed herein) is administered at a volume of about 0.01 to 2.00 mL, about 0.02 to 2.00 mL, about 0.03 to 2.00 mL, about 0.04 to 2.00 mL, about 0.05 to 2.00 mL, about 0.06 to 2.00 mL, about 0.07 to 2.00 mL, about 0.08 to 2.00 mL, about 0.09 to 2.00 mL, about 0.01 to 1.90 mL, about 0.01 to 1.80 mL, about 0.01 to 1.70 mL, about 0.01 to 1.60 mL, about 0.01 to 1.50 mL, about 0.01 to 1.40 mL, about 0.01 to 1.30 mL, about 0.01 to 1.20 mL, about 0.01 to 1.10 mL, about 0.01 to 1.00 mL, about 0.01 to 0.09 mL.

In some aspects, a single dose comprises about 0.01 mL-0.2 mL. In some aspects, a single dose comprises about 0.01-0.2, about 0.02-0.18, about 0.03-0.16, about 0.04-0.14, about 0.05-0.13, about 0.06-0.12, about 0.07-0.11, or about 0.08-0.10 mL. In some aspects, a single dose comprises about 0.01, about 0.02, about 0.03, about 0.04, about 0.05, about 0.06, about 0.07, about 0.08, about 0.09, about 0.1, about 0.11, about 0.012, about 0.13, about 0.14, about 0.15, about 0.16, about 0.17, about 0.18, about 0.19, or about 0.2 mL. In some aspects, a single dose comprises about 0.09 mL.

### Formulations

Also provided herein are formulations comprising any of the compositions herein.

In some aspects, compositions of the disclosure comprise a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition comprises about 4.1E10-8.1E12 total vg.

In some aspects, compositions of the disclosure comprise a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition comprises about 4.1E10-4.1E12 total vg or about 8.1E10-8.1E12 total vg.

In some aspects, compositions of the disclosure comprise (a) a first rAAV vector genome comprising a first expression cassette comprising a promoter, a first coding sequence that encodes an N-terminal portion of an otoferlin protein positioned 3′ of the promoter, and a splicing donor signal sequence positioned at the 3′ end of the first coding sequence; and (b) a second rAAV vector genome comprising a second expression cassette comprising a splicing acceptor signal sequence, a second coding sequence that encodes a C-terminal portion of an otoferlin protein positioned at the 3′ end of the splicing acceptor signal sequence, and a polyadenylation sequence at 3′ end of the second coding sequence, wherein the composition is formulated for intra-cochlear administration.

In some aspects, compositions of the disclosure comprise a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and b) a second rAAV vector genome comprising a second expression cassette comprising a nucleic acid sequence comprising a 3′ portion of an otoferlin gene, wherein the expression cassette is flanked by ITRs; wherein the composition is formulated for intra-cochlear administration. In some aspects, the composition comprises the first rAAV vector genome and the second rAAV vector genome in about a 1:1 ratio.

In some aspects, the composition comprises about 4.1E10-8.1E12 total vg. In some aspects, the composition comprises about 4.1E10-4.1E12 total vg or about 8.1E10-8.1E12 total vg.

In some aspects, the composition comprises about 4.1E10-4.1E12, about 5.1E10-3.1E12, about 6.1E10-2.1E12, about 7.1E10-1.1E12, about 8.1E10-10.1E11, about 9.1E10-9.1E11, about 10.1E11-8.1E11, about 1.1E11-7.1E11, about 2.1E11-6.1E11, or about 3.1E11-5.1E11 total vg. In some aspects, the composition comprises about 3.1E11-5.1E11, about 3.2E11-5.0E11, about 3.3E11-4.9E11, about 3.4E11-4.8E11, about 3.5E11-4.7E11, about 3.6E11-4.6E11, about 3.7E11-4.5E11, about 3.8E11-4.4E11, about 3.9E11-4.3E11, or about 4.0E11-4.2E11 total vg. In some aspects, the composition comprises about 4.1E11 total vg.

In some aspects, the composition comprises about 4.1E10-8.1E12 total vg/cochlea. In some aspects, the composition comprises about 4.1E10-4.1E12 total vg/cochlea or about 8.1E10-8.1E12 total vg/cochlea.

In some aspects, the composition comprises about 4.1E10-4.1E12, about 5.1E10-3.1E12, about 6.1E10-2.1E12, about 7.1E10-1.1E12, about 8.1E10-10.1E11, about 9.1E10-9.1E11, about 10.1E11-8.1E11, about 1.1E11-7.1E11, about 2.1E11-6.1E11, or about 3.1E11-5.1E11 total vg/cochlea. In some aspects, the composition comprises about 3.1E11-5.1E11, about 3.2E11-5.0E11, about 3.3E11-4.9E11, about 3.4E11-4.8E11, about 3.5E11-4.7E11, about 3.6E11-4.6E11, about 3.7E11-4.5E11, about 3.8E11-4.4E11, about 3.9E11-4.3E11, or about 4.0E11-4.2E11 total vg/cochlea. In some aspects, the composition comprises about 4.1E11 total vg/cochlea.

In some aspects, the composition comprises about 3.0E11, about 3.1E11, about 3.2E11, about 3.3E11, about 3.4E11, about 3.5E11, about 3.6E11, about 3.7E11, about 3.8E11, about 3.9E11, about 4.0E11, about 4.1E11, about 4.2E11, about 4.3E11, about 4.4E11, about 4.5E11, about 4.6E11, about 4.7E11, about 4.8E11, about 4.9E11, about 5.0E11, about 5.1E11, about 5.2E11, about 5.3E11, about 5.4E11, about 5.5E11, about 5.6E11, about 5.7E11, about 5.8E11, about 5.9E11, about 6.0E11, about 6.1E11, about 6.2E11, about 6.3E11, about 6.4E11, about 6.5E11, about 6.6E11, about 6.7E11, about 6.8E11, about 6.9E11, about 7.0E11, about 7.1E11, about 7.2E11, about 7.3E11, about 7.4E11, about 7.5E11, about 7.6E11, about 7.7E11, about 7.8E11, about 7.9E11, about 8.0E11, about 8.1E11, about 8.2E11, about 8.3E11, about 8.4E11, about 8.5E11, about 8.6E11, about 8.7E11, about 8.8E11, or about 8.9E11 total vg.

In some aspects, the composition comprises about 3.0E11, about 3.1E11, about 3.2E11, about 3.3E11, about 3.4E11, about 3.5E11, about 3.6E11, about 3.7E11, about 3.8E11, about 3.9E11, about 4.0E11, about 4.1E11, about 4.2E11, about 4.3E11, about 4.4E11, about 4.5E11, about 4.6E11, about 4.7E11, about 4.8E11, about 4.9E11, about 5.0E11, about 5.1E11, about 5.2E11, about 5.3E11, about 5.4E11, about 5.5E11, about 5.6E11, about 5.7E11, about 5.8E11, about 5.9E11, about 6.0E11, about 6.1E11, about 6.2E11, about 6.3E11, about 6.4E11, about 6.5E11, about 6.6E11, about 6.7E11, about 6.8E11, about 6.9E11, about 7.0E11, about 7.1E11, about 7.2E11, about 7.3E11, about 7.4E11, about 7.5E11, about 7.6E11, about 7.7E11, about 7.8E11, about 7.9E11, about 8.0E11, about 8.1E11, about 8.2E11, about 8.3E11, about 8.4E11, about 8.5E11, about 8.6E11, about 8.7E11, about 8.8E11, or about 8.9E11 total vg/cochlea.

In some aspects, the composition comprises about 8.1E10-8.1E12, about 9.1E10-7.1E12, about 10.1E10-6.1E12, about 1.1E11-5.1E12, about 2.1E11-4.1E12, about 3.1E11-3.1E12, about 4.1E11-2.1E12, about 5.1E11-1.1E12, about 6.1E11-10.1E11, or about 7.1E11-9.1E11 total vg. In some aspects, the composition comprises about 7.1E11-9.1E11, about 7.2E11-9.0E11, about 7.3E11-8.9E11, about 7.4E11-8.8E11, about 7.5E11-8.7E11, about 7.6E11-8.6E11, about 7.7E11-8.5E11, about 7.8E11-8.4E11, about 7.9E11-8.3E11, or about 8.0E11-8.2E11 total vg. In some aspects, the composition comprises about 8.1E11 total vg.

In some aspects, the composition comprises about 8.1E10-8.1E12, about 9.1E10-7.1E12, about 10.1E10-6.1E12, about 1.1E11-5.1E12, about 2.1E11-4.1E12, about 3.1E11-3.1E12, about 4.1E11-2.1E12, about 5.1E11-1.1E12, about 6.1E11-10.1E11, or about 7.1E11-9.1E11 total vg/cochlea. In some aspects, the composition comprises about 7.1E11-9.1E11, about 7.2E11-9.0E11, about 7.3E11-8.9E11, about 7.4E11-8.8E11, about 7.5E11-8.7E11, about 7.6E11-8.6E11, about 7.7E11-8.5E11, about 7.8E11-8.4E11, about 7.9E11-8.3E11, or about 8.0E11-8.2E11 total vg/cochlea. In some aspects, the composition comprises about 8.1E11 total vg/cochlea.

In some aspects, the concentration of the composition comprises about 4.5E11-9E13 total vg/mL. In some aspects, the concentration of the composition comprises about 4.5E11-4.5E13 total vg/mL or about 9E11-9E13 total vg/mL. In some aspects, the concentration of the composition comprises about 4.5E11-4.5E13 total vg/mL. In some aspects, the concentration of the composition comprises about 5.5E11-3.5E13, about 6.5E11-2.5E13, about 7.5E11-1.5E13, about 8.5E11-10.5E12, about 9.5E11-9.5E12, about 10.5E11-8.5E12, about 1.5E12-7.5E12, about 2.5E12-6.5E12, or about 3.5E12-5.5E12 total vg/mL. In some aspects, the concentration of the composition comprises about 3.5E12-5.5E12, about 3.6E12-5.4E12, about 3.7E12-5.3E12, about 3.8E12-5.2E12, about 3.9E12-5.1E12, about 4.0E12-5.0E12, about 4.1E12-4.9E12, about 4.2E12-4.8E12, about 4.3E12-4.7E12, or about 4.4E12-4.6E12 total vg/mL. In some aspects, the concentration of the composition comprises about 4.5E12 total vg/mL.

In some aspects, the concentration of the composition comprises about 3.5E12, about 3.6E12, about 3.7E12, about 3.8E12, about 3.9E12, about 4.0E12, about 4.1E12, about 4.2E12, about 4.3E12, about 4.4E12, about 4.5E12, about 4.6E12, about 4.7E12, about 4.8E12, about 4.9E12, about 5.0E12, about 5.1E12, about 5.2E12, about 5.3E12, about 5.4E12, about 5.5E12, about 5.6E12, about 5.7E12, about 5.8E12, about 5.9E12, about 6.0E12, about 6.1E12, about 6.2E12, about 6.3E12, about 6.4E12, about 6.5E12, about 6.6E12, about 6.7E12, about 6.8E12, about 6.9E12, about 7.0E12, about 7.1E12, about 7.2E12, about 7.3E12, about 7.4E12, about 7.5E12, about 7.6E12, about 7.7E12, about 7.8E12, about 7.9E12, about 8.0E12, about 8.1E12, about 8.2E12, about 8.3E12, about 8.4E12, about 8.5E12, about 8.6E12, about 8.7E12, about 8.8E12, about 8.9E12, about 9.0E12, about 9.1E12, about 9.2E12, about 9.3E12, about 9.4E12, about 9.5E12, about 9.6E12, about 9.7E12, about 9.8E12, or about 9.9E12 total vg/mL.

In some aspects, the concentration of the composition comprises about 9E11-9E13 total vg/mL. In some aspects, the concentration of the composition comprises about 9E11-9E13, about 10E11-8E13, about 1E12-7E13, about 2E12-6E13, about 3E12-5E13, about 4E12-4E13, about 5E12-3E13, about 6E12-2E13, about 7E12-1E13, or about 8E12-10E12 total vg/mL. In some aspects, the concentration of the composition comprises about 8E12-10E12, about 8.1E12-9.9E12, about 8.2E12-9.8E12, about 8.3E12-9.7E12, about 8.4E12-9.6E12, about 8.5E12-9.5E12, about 8.6E12-9.4E12, about 8.7E12-9.3E12, about 8.8E12-9.2E12, or about 8.9E12-9.1E12. In some aspects, the concentration of the composition comprises 9E12 total vg/mL.

Pharmaceutical compositions of the present disclosure may comprise the rAAV vectors, as described herein, in combination with one or more pharmaceutically or physiologically acceptable carriers, diluents, or excipients.

In some aspects, the composition may comprise buffering agents such as neutral buffered saline, phosphate buffered saline, monopotassium phosphate, sodium phosphate dibasic, potassium chloride, sodium chloride, Tris HCl, Tris base, histidine, boric acid, citric acid, glycine, HEPES, and MOPS; carbohydrates such as glucose, mannose, sucrose, or dextrans; mannitol; proteins; polypeptides or amino acids such as glycine; antioxidants; chelating agents such as EDTA or glutathione; adjuvants (e.g., aluminum hydroxide); surfactants such as poloxamer 188, labrasol, tween, ethanol, pluronic F68, and polyethyelene glycol; and preservatives.

In some aspects, the composition comprises one or more buffering agents and one or more surfactants. In some aspects, the buffering agents are selected from monopotassium phosphate, sodium phosphate dibasic, potassium chloride, sodium chloride, Tris HCl, Tris base, histidine, boric acid, citric acid, glycine, HEPES, and MOPS. In some aspects, the surfactant is selected from poloxamer 188, labrasol, tween, ethanol, pluronic F68, and polyethyelene glycol.

In some aspects, the composition comprises monopotassium phosphate, sodium phosphate dibasic, potassium chloride, sodium chloride, and Poloxamer 188. In some aspects, the composition comprises a) about 1.35-1.65 mM monopotassium phosphate; b) about 7.29-8.91 mM sodium phosphate dibasic; c) about 2.43-2.97 mM potassium chloride; d) about 154.8-189.2 mM sodium chloride; and e) about 0.0001%-0.01% Poloxamer 188.

In some aspects, the composition comprises a) about 1.35, about 1.375, about 1.4, about 1.425, about 1.45, about 1.475, about 1.5, about 1.525, about 1.55, about 1.575, about 1.6, about 1.625, or about 1.65 mM monopotassium phosphate; b) about 7.29, about 7.4, about 7.5 about 7.6, about 7.7 about 7.8, about 7.9, about 8.0, about 8.1, about 8.2, about 8.3, about 8.4, about 8.5, about 8.6, about 8.7, about 8.8, or about 8.91 mM sodium phosphate dibasic; c) about 2.43, about 2.5, about 2.55, about 2.6, about 2.65, about 2.7, about 2.75, about 2.8, about 2.85, about 2.9, about 2.95, or about 2.97 mM potassium chloride; d) about 154.8, about 160, about 165, about 170, about 175, about 180, about 185, or about 189.2 mM sodium chloride, and e) about 0.0001%, about 0.00025% about 0.0005%, about 0.00075%, about 0.001%, about 0.0025%, about 0.005%, about 0.0075%, or about 0.01% poloxamer 188.

In some aspects, the composition comprises a) about 1.5 mM monopotassium phosphate; b) about 8.1 mM sodium phosphate dibasic; c) about 2.7 mM potassium chloride; d) about 172 mM sodium chloride; and e) about 0.001% Poloxamer 188.

In some aspects, the composition is formulated as a sterile suspension. In some aspects, the sterile suspension comprises a pharmaceutically acceptable carrier. In some aspects, the suspension comprises sterile water. In some aspects, the composition comprises a volume of about 0.01 mL to 0.2 mL. In some aspects, the composition comprises a volume of about 0.01-0.2, about 0.02-0.18, about 0.03-0.16, about 0.04-0.14, about 0.05-0.13, about 0.06-0.12, about 0.07-0.11, about 0.08-0.10 mL. In some aspects, a single dose comprises about 0.01, about 0.02, about 0.03, about 0.04, about 0.05, about 0.06, about 0.07, about 0.08, about 0.09, about 0.1, about 0.11, about 0.012, about 0.13, about 0.14, about 0.15, about 0.16, about 0.17, about 0.18, about 0.19, or about 0.2 mL. In some aspects, the composition comprises a volume of about 0.09 mL.

In some aspects, the compositions of the present disclosure are formulated for intra-cochlear administration. In some aspects, the compositions of the present disclosure are formulated for intravenous administration.

In some aspects, the compositions are formulated to comprise a synthetic perilymph solution. In some aspects, the synthetic perilymph solution comprises one or more buffering agents and one or more surfactants. In some aspects, the buffering agents are selected from monopotassium phosphate, sodium phosphate dibasic, potassium chloride, sodium chloride, Tris HCl, Tris base, histidine, boric acid, citric acid, glycine, HEPES, and MOPS. In some aspects, the surfactant is selected from poloxamer 188, labrasol, tween, ethanol, pluronic F68, and polyethyelene glycol.

In some aspects, the synthetic perilymph solution comprises monopotassium phosphate, sodium phosphate dibasic, potassium chloride, sodium chloride, and Poloxamer 188. In some aspects, the synthetic perilymph solution comprises a) about 1.35-1.65 mM monopotassium phosphate; b) about 7.29-8.91 mM sodium phosphate dibasic; c) about 2.43-2.97 mM potassium chloride; d) about 154.8-189.2 mM sodium chloride; and e) about 0.0001%-0.01% Poloxamer 188.

In some aspects, the synthetic perilymph solution comprises a) about 1.35, about 1.375, about 1.4, about 1.425, about 1.45, about 1.475, about 1.5, about 1.525, about 1.55, about 1.575, about 1.6, about 1.625, or about 1.65 mM monopotassium phosphate; b) about 7.29, about 7.4, about 7.5 about 7.6, about 7.7 about 7.8, about 7.9, about 8.0, about 8.1, about 8.2, about 8.3, about 8.4, about 8.5, about 8.6, about 8.7, about 8.8, or about 8.91 mM sodium phosphate dibasic; c) about 2.43, about 2.5, about 2.55, about 2.6, about 2.65, about 2.7, about 2.75, about 2.8, about 2.85, about 2.9, about 2.95, or about 2.97 mM potassium chloride; d) about 154.8, about 160, about 165, about 170, about 175, about 180, about 185, or about 189.2 mM sodium chloride, and e) about 0.0001%, about 0.00025% about 0.0005%, about 0.00075%, about 0.001%, about 0.0025%, about 0.005%, about 0.0075%, or about 0.01% poloxamer 188.

In some aspects, the synthetic perilymph solution comprises a) about 1.5 mM monopotassium phosphate; b) about 8.1 mM sodium phosphate dibasic; c) about 2.7 mM potassium chloride; d) about 172 mM sodium chloride; and e) about 0.001% Poloxamer 188.

In some aspects, the compositions described herein can further include one or more agents that promote the entry of a nucleic acid or any of the vectors described herein into a mammalian cell (e.g., a liposome or cationic lipid). In some aspects, any of the vectors described herein can be formulated using natural and/or synthetic polymers. Non-limiting examples of polymers that may be included in any of the compositions described herein can include, but are not limited to, DYNAMIC POLYCONJUGATE® (Arrowhead Research Corp., Pasadena, Calif.), formulations from Mirus Bio (Madison, Wis.) and Roche Madison (Madison, Wis.), PhaseRX polymer formulations such as, without limitation, SMARTT POLYMER TECHNOLOGY® (PhaseRX, Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly(lactic-co-glycolic acid) (PLGA) polymers, RONDEL™ (RNAi/Oligonucleotide Nanoparticle Delivery) polymers (Arrowhead Research Corporation, Pasadena, Calif.), and pH responsive co-block polymers, such as, but not limited to, those produced by PhaseRX (Seattle, Wash.). Many of these polymers have demonstrated efficacy in delivering oligonucleotides in vivo into a mammalian cell (see, e.g., deFougerolles, Human Gene Ther. 19:125-132, 2008; Rozema et al., Proc. Natl. Acad. Sci. U.S.A. 104:12982-12887, 2007; Rozema et al., Proc. Natl. Acad. Sci. U.S.A. 104:12982-12887, 2007; Hu-Lieskovan et al., Cancer Res. 65:8984-8982, 2005; Heidel et al., Proc. Natl. Acad. Sci. U.S.A. 104:5715-5721, 2007). Any of the compositions described herein can be, e.g., a pharmaceutical composition.

In some aspects, the composition includes a pharmaceutically acceptable carrier (e.g., phosphate buffered saline, saline, or bacteriostatic water). Upon formulation, solutions will be administered in a manner compatible with the dosage formulation and in such amount as is therapeutically effective. The formulations are easily administered in a variety of dosage forms such as injectable solutions, injectable gels, drug-release capsules, and the like.

The compositions provided herein can be, e.g., formulated to be compatible with their intended route of administration. A non-limiting example of an intended route of administration is local administration (e.g., intra-cochlear administration).

Also provided are kits including any of the compositions described herein. In some aspects, a kit can include a solid composition (e.g., a lyophilized composition including the at least two different vectors described herein) and a liquid for solubilizing the lyophilized composition. In some aspects, a kit can include a pre-loaded syringe including any of the compositions described herein.

In some aspects, a kit can include the composition is pre-loaded in a device. In some aspects, the device is a microcatheter. In some aspects, the microcatheter is shaped such that it can enter the middle ear cavity via the external auditory canal and contact the end of the microcatheter with the RWM. In some aspects, a distal end of the microcatheter is comprised of at least one microneedle with diameter of between 10 and 1,000 microns.

In some aspects, a kit can include a device. In some aspects, the device is a device provided herein. In some aspects, the device is a device described in any one of FIGS. 2-5. In some aspects, the device comprises a needle comprising a bent portion and an angled tip.

In some aspects, a kit includes a vial comprising any of the compositions described herein (e.g., formulated as an aqueous composition, e.g., an aqueous pharmaceutical composition). In some aspects, the vial is a single use vial or multi-use vial.

In some aspects, the vial is a single-use vial. In some aspects, the single-use vial comprises the composition at a concentration appropriate for intra-cochlear administration (target concentration). In some aspects, the target concentration is about 4.5E11-9E13 vg/mL. In some aspects, the target concentration is about 1E13 total vg/mL.

In some aspects, the kit includes a second vial comprising a diluent. In some aspects, the second vial is a single-use vial or a multi-use vial. In some aspects, the diluent comprises the one or more pharmaceutically or physiologically acceptable carriers, diluents, or excipients of the composition. In some aspects, the concentrations of the one or more pharmaceutically or physiologically acceptable carriers, diluents, or excipients of the composition are equivalent to the concentrations of the one or more pharmaceutically or physiologically acceptable carriers, diluents, or excipients comprised in the composition. In some aspects, the diluent comprises a) about 1.5 mM monopotassium phosphate; b) about 8.1 mM sodium phosphate dibasic; c) about 2.7 mM potassium chloride; d) about 172 mM sodium chloride; and e) about 0.001% Poloxamer 188. In some aspects, the diluent is utilized to prepare the concentration of the composition to be administered to a subject in need thereof.

In some aspects, a kit can include instructions for performing any of the methods described herein. The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

The administration of the subject compositions may be carried out in any convenient manner, including by aerosol inhalation, injection, ingestion, transfusion, implantation or transplantation. The compositions described herein may be administered to a subject trans arterially, subcutaneously, intradermally, intranodally, intramedullary, intramuscularly, by intravenous (i.v.) injection, or intraperitoneally. In one aspect, the nucleic acid compositions of the present disclosure are administered to a subject by intradermal or subcutaneous injection. In one aspect, the nucleic compositions of the present disclosure are administered by i.v. injection.

### Devices and Surgical Methods

Provided herein are technologies (e.g., systems, methods, devices, etc.) that may be used, in some aspects, for treating deafness and other hearing-associated diseases, disorders and conditions. Examples of such technologies are also included in, e.g., WO2017223193 and WO2019084145, each of which is herein incorporated by reference in its entirety. In one aspect, the present disclosure provides therapeutic delivery systems for treating deafness and other hearing-associated diseases, disorders and conditions. In one aspect, provided are therapeutic delivery systems that include i) a medical device capable of creating one or a plurality of incisions in a round window membrane of an inner ear of a human subject in need thereof, and ii) an effective dose of a therapeutic composition comprising one or a plurality of adeno-associated viral (AAV) vectors, wherein the one or the plurality of AAV vectors are capable of constituting a full-length auditory polypeptide messenger RNA in a target cell of the inner ear. In some aspects, of a means for performing a surgical method, the method comprises the steps of: administering intra-cochlearly to a human subject in need thereof an effective dose of a therapeutic composition of the present disclosure, wherein the therapeutic composition is capable of being administered by using a medical device which comprises: a) a means for creating one or a plurality of incisions in a round window membrane; and b) an effective dose of a therapeutic composition.

Provided herein are surgical methods for treatment of hearing loss. In one aspect, the methods include the steps of: introducing into a cochlea of a human subject a first incision at a first incision point; and administering intra-cochlearly an effective dose of a therapeutic composition (e.g., any of the compositions described herein) as provided herein. In one aspect, a therapeutic composition (e.g., any of the compositions described herein) is administered to the subject at a first incision point. In one aspect, a therapeutic composition is administered to a subject into or through a first incision. In one aspect, a therapeutic composition is administered to a subject into or through a cochlea oval window membrane. In one aspect, a therapeutic composition is administered to a subject into or through a cochlea round window membrane.

In some aspects, a composition disclosed herein can be administered to a subject with a surgical procedure. In some aspects, administration, e.g., via a surgical procedure, comprises injecting a composition disclosed herein via a delivery device as described herein into the inner ear. In some aspects, a surgical procedure disclosed herein comprises performing a transcanal tympanotomy; performing a laser-assisted micro-stapedotomy; and injecting a composition disclosed herein via a delivery device as described herein into the inner ear.

In some aspects, a surgical procedure comprises performing a transcanal tympanotomy; performing a laser-assisted micro-stapedotomy; injecting a composition disclosed herein via a delivery device as described herein into the inner ear; applying sealant around the round window and/or an oval window of the subject; and lowering a tympanomeatal flap of the subject to the anatomical position.

In some aspects, a surgical procedure comprises performing a transcanal tympanotomy; preparing a round window of the subject; performing a laser-assisted micro-stapedotomy; preparing both a delivery device as described herein and a composition disclosed herein for delivery to the inner ear; injecting a composition disclosed herein via the delivery device into the inner ear; applying sealant around the round window and/or an oval window of the subject; and lowering a tympanomeatal flap of the subject to the anatomical position.

In some aspects, performing a laser-assisted micro-stapedotomy includes using a KTP otologic laser and/or a CO2 otologic laser.

In some embodiments, a composition comprises one or a plurality of AAV vectors. In some aspects, when more than one AAV vector is included in the composition, the AAV vectors are each different. In some aspects, an AAV vector comprises an OTOF coding region, e.g., as described herein. In some aspects, a composition comprises an rAAV particle comprising an AAV vector described herein. In some aspects, the r AAV particle is encapsidated by an Anc80 capsid. In some aspect, the Anc80 capsid comprises a polypeptide of SEQ ID NO: 109.

For example, in one aspect, a therapeutic composition is administered using a medical device capable of creating a plurality of incisions in a round window membrane. In one aspect, a medical device includes a plurality of micro-needles. In one aspect, a medical device includes a plurality of micro-needles including a generally circular first aspect, wherein each micro-needle has a diameter of at least about 10 microns. In one aspect, a medical device includes a base and/or a reservoir capable of holding a therapeutic composition. In one aspect, a medical device includes a plurality of hollow micro-needles individually including a lumen capable of transferring a therapeutic composition. In one aspect, a medical device includes a means for generating at least a partial vacuum.

As another example, a composition disclosed herein is administered using a device and/or system specifically designed for intracochlear route of administration. In some aspects, design elements of a device described herein may include: maintenance of sterility of injected fluid; minimization of air bubbles introduced to the inner ear; ability to precisely deliver small volumes at a controlled rate; delivery through the external auditory canal by the surgeon; minimization of damage to the round window membrane (RWM), or to inner ear, e.g., cochlear structures beyond the RWM; and/or minimization of injected fluid leaking back out through the RWM.

The devices, systems, and methods provided herein also describe the potential for delivering a composition safely and efficiently into the inner ear, in order to treat conditions and disorders that would benefit from delivery of a composition disclosed herein to the inner ear, including, but not limited to, hearing disorders, e.g., as described herein. As another example, by placing a vent in the stapes footplate and injecting through the RWM, a composition disclosed herein is dispersed throughout the cochlea with minimal dilution at the site of action. The development of the described devices allows the surgical administration procedure to be performed through the external auditory canal in humans. The described devices can be removed from the ear following infusion of an amount of fluid into the perilymph of the cochlea. In subjects, the device may be advanced through the external auditory canal, either under surgical microscopic control or along with an endoscope.

An exemplary device for use in any of the methods disclosed herein is described in FIGS. 2-5. FIG. 2 illustrates an exemplary device 10 for delivering fluid to an inner ear. Device 10 includes a knurled handle 12, and a distal handle adhesive 14 (for example, an epoxy such as loctite 4014) that couples to a telescoping hypotube needle support 24. The knurled handle 12 (or handle portion) may include kurling features and/or grooves to enhance the grip. The knurled handle 12 (or handle portion) may be from about 5 mm to about 15 mm thick or from about 5 mm to about 12 mm thick, or from about 6 mm to about 10 mm thick, or from about 6 mm to about 9 mm thick, or from about 7 mm to about 8 mm thick. The knurled handle 12 (or handle portion) may be hollow such that fluid may pass through the device 10 during use. The device 10 may also include a proximal handle adhesive 16 at a proximal end 18 of the knurled handle 12, a needle sub-assembly 26 (shown in FIG. 3) with stopper 28 (shown in FIG. 3) at a distal end 20 of the device 10, and a strain relief feature 22. Strain relief feature 22 may be composed of a Santoprene material, a Pebax material, a polyurethane material, a silicone material, a nylon material, and/or a thermoplastic elastomer. The telescoping hypotube needle support 24 surrounds and supports a bent needle 38 (shown in FIG. 3) disposed therewithin.

Referring still to FIG. 2, the stopper 28 may be composed of a thermoplastic material or plastic polymer (such as a UV-cured polymer), as well as other suitable materials, and may be used to prevent the bent needle 38 from being inserted too far into the ear canal (for example, to prevent insertion of bent needle 38 into the lateral wall or other inner ear structure). Device 10 also may include a tapered portion 23 disposed between the knurled handle 12 and the distal handle adhesive 14 that is coupled to the telescoping hypotube needle support 24. The knurled handle 12 (or handle portion) may include the tapered portion 23 at the distal end of the handle portion 12. Device 10 may also include tubing 36 fluidly connected to the proximal end 16 the device 10 and acts as a fluid inlet line connecting the device to upstream components (for example, a pump, a syringe, and/or upstream components which, in some embodiments, may be coupled to a control system and/or power supply (not shown)). In some aspects, the bent needle 38 (shown in FIG. 3) extends from the distal end 20, through the telescoping hypotube needle support 24, through the tapered portion 23, through the knurled handle 12, and through the strain relief feature 22 and fluidly connects directly to the tubing 36. In other aspects, the bent needle 38 fluidly connects with the hollow interior of the knurled handle (for example, via the telescoping hypotube needle support 24) which in turn fluidly connects at a proximal end 16 with tubing 36. In aspects where the bent needle 38 does not extend all the way through the interior of the device 10, the contact area (for example, between overlapping nested hyotubes 42), the tolerances, and/or sealants between interfacing components must be sufficient to prevent therapeutic fluid from leaking out of the device 10 (which operates at a relatively low pressure (for example, from about 1 Pascal to about 50 Pa, or from about 2 Pa to about 20 Pa, or from about 3 Pa to about 10 Pa)).

FIG. 3 illustrates a sideview of the bent needle sub-assembly 26, according to aspects of the present disclosed aspects. Bent needle sub-assembly 26 includes a needle 38 that has a bent portion 32. Bent needle sub-assembly 26 may also include a stopper 28 coupled to the bent portion 32. The bent portion 32 includes an angled tip 34 at the distal end 20 of the device 10 for piercing a membrane of the ear (for example, the RWM). The needle 38, bent portion 32, and angled top 34 are hollow such that fluid may flow therethrough. The angle 46 (as shown in FIG. 5) of the bent portion 32 may vary. A stopper 28 geometry may be cylindrical, disk-shaped, annulus-shaped, dome-shaped, and/or other suitable shapes. Stopper 28 may be molded into place onto bent portion 32. For example, stopper 28 may be positioned concentrically around the bent portion 32 using adhesives or compression fitting. Examples of adhesives include an UV cure adhesive (such as Dymax 203A-CTH-F-T), elastomer adhesives, thermoset adhesives (such as epoxy or polyurthethane), or emulsion adhesives (such as polyvinyl acetate). Stopper 28 fits concentrically around the bent portion 32 such that angled tip 34 is inserted into the ear at a desired insertion depth. The bent needle 38 may be formed from a straight needle using incremental forming, as well as other suitable techniques.

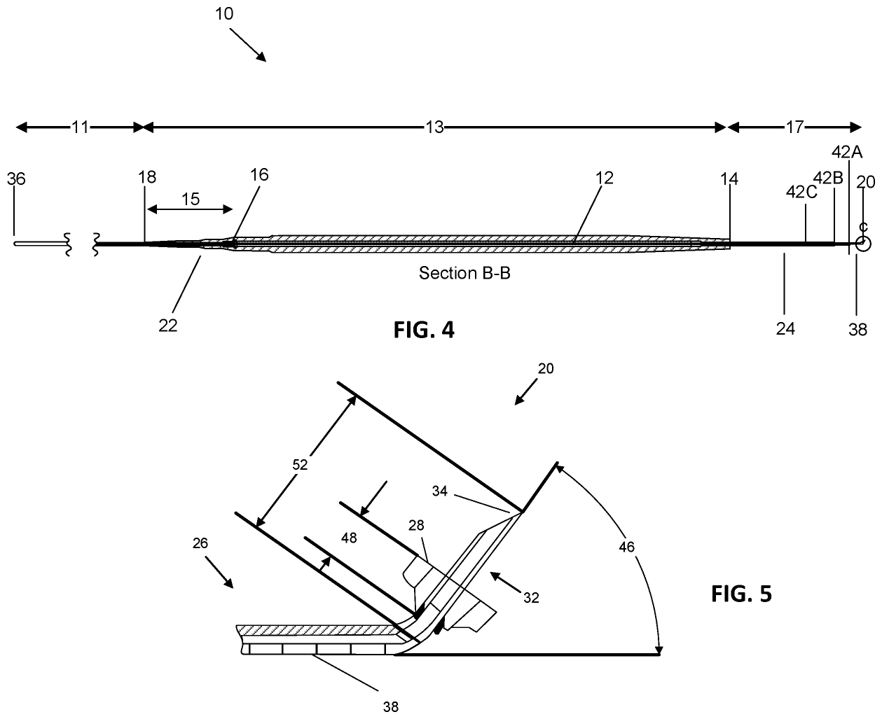

FIG. 4 illustrates a perspective view of exemplary device 10 for delivering fluid to an inner ear. Tubing 36 may be from about 1300 mm in length (dimension 11 in FIG. 4) to about 1600 mm, or from about 1400 mm to about 1500 mm, or from about 1430 mm to about 1450 mm. Strain release feature 22 may be from about 25 mm to about 30 mm in length (dimension 15 in FIG. 4), or from about 20 mm to about 35 mm in length. Handle 12 may be about 155.4 mm in length (dimension 13 in FIG. 4), or from about 150 mm to about 160 mm, or from about 140 mm to about 170 mm. The telescoping hypotube needle support 24 may have two or more nested hypotubes, for example three nested hypotubes 42A, 42B, and 42C, or four nested hypotubes 42A, 42B, 42C, and 42D. The total length of hypotubes 42A, 42B, 42C and tip assembly 26 (dimension 17 in FIG. 4) may be from about 25 mm to about 45 mm, or from about 30 mm to about 40 mm, or about 35 mm. In addition, telescoping hypotube needle support 24 may have a length of about 36 mm, or from about 25 mm to about 45 mm, or form about 30 mm to about 40 mm. The three nested hypotubes 42A, 42B, and 42C each may have a length of 3.5 mm, 8.0 mm, and 19.8 mm, respectively, plus or minus about 20%. The inner-most nested hypotube (or most narrow portion) of the telescoping hypotube needle support 24 may be concentrically disposed around needle 38.

FIG. 5 illustrates a perspective view of bent needle sub-assembly 26 coupled to the distal end 20 of device 10, according to aspects of the present disclosed aspects. As shown in FIG. 5, bent needle sub-assembly 26 may include a needle 38 coupled to a bent portion 32. In other aspects, the bent needle 38 may be a single needle (for example, a straight needle that is then bent such that it includes the desired angle 46). Needle 38 may be a 33-gauge needle, or may include a gauge from about 32 to about 34, or from about 31 to 35. At finer gauges, care must be taken to ensure tubing 36 is not kinked or damaged. Needle 38 may be attached to handle 12 for safe and accurate placement of needle 38 into the inner ear. As shown in FIG. 5, bent needle sub-assembly 26 may also include a stopper 28 disposed around bent portion 32. FIG. 5 also shows that bent portion 32 may include an angled tip 34 for piercing a membrane of the ear (for example, the RWM). Stopper 28 may have a height 48 of about 0.5 mm, or from about 0.4 mm to about 0.6 mm, or from about 0.3 mm to about 0.7 mm. Bent portion 32 may have a length 52 of about 1.45 mm, or from about 1.35 mm to about 1.55 mm, or from about 1.2 mm to about 1.7 mm. In other aspects, the bent portion 32 may have a length greater than 2.0 mm such that the distance between the distal end of the stopper 28 and the distal end of the angled tip 34 is from about 0.5 mm to about 1.7 mm, or from about 0.6 mm to about 1.5 mm, or from about 0.7 mm to about 1.3 mm, or from about 0.8 mm to about 1.2 mm. FIG. 5 shows that stopper 28 may have a geometry that is cylindrical, disk-shaped, and/or dome-shaped. A person of ordinary skill will appreciate that other geometries could be used.

The disclosure is further described in detail by reference to the following experimental examples. These examples are provided for purposes of illustration only, and are not intended to be limiting unless otherwise specified. Thus, the disclosure should in no way be construed as being limited to the following examples, but rather should be construed to encompass any and all variations that become evident as a result of the teaching provided herein.

Other assays, including those described in the Example section herein as well as those that are known in the art, can also be used to evaluate the auditory polypeptide nucleic acids and nucleic acid constructs of the disclosure.

Without further description, it is believed that one of ordinary skill in the art can, using the preceding description and the following illustrative examples, make and utilize the compounds of the present disclosure and practice the claimed methods. The following working examples specifically point out various aspects of the present disclosure, and are not to be construed as limiting in any way the remainder of the disclosure.

### EXAMPLES

### Example 1: Adeno-Associated Virus (AAV) Trans-Splicing Strategy

Two different rAAV vectors can be used to reconstitute an active otoferlin gene (e.g., a full-length otoferlin gene) within a cell following intermolecular concatamerization and trans-splicing. See, e.g., Yan et al., Proc. Natl. Acad. Sci. U.S.A. 97:12; 6716-6721, 2000, incorporated in its entirety herein.

In some aspects, two different rAAV vectors will be used. In some aspects, the first rAAV vector comprises a first expression cassette comprising a promoter (e.g., any of the promoters described herein), a first coding sequence that encodes an N-terminal portion of an otoferlin protein positioned 3′ of the promoter (e.g., any of the sizes of a portion of an otoferlin protein described herein and/or any of the N-terminal portions of an otoferlin protein described herein), and a splicing donor signal sequence positioned at the 3′ end of the first coding sequence.

In some aspects, the second rAAV vector comprises a second expression cassette comprising a splicing acceptor signal sequence, a second coding sequence that encodes a C-terminal portion of an otoferlin protein (i.e., the entire portion of the otoferlin protein that is not included in the N-terminal portion) positioned at the 3′ end of the splicing acceptor signal sequence (e.g., any of the sizes of a portion of an otoferlin protein described herein and/or any of the C-terminal portions of an otoferlin protein described herein), and a polyadenylation sequence at 3′ end of the second coding sequence (e.g., any of the polyadenylation sequences described herein).

In some aspects, the first rAAV vector genome comprises a first expression cassette comprising a promoter (e.g., any of the promoters described herein), a first coding sequence that encodes an N-terminal portion of an otoferlin protein positioned 3′ of the promoter (e.g., any of the sizes of a portion of an otoferlin protein described herein and/or any of the N-terminal portions of an otoferlin protein described herein), and a splicing donor signal sequence positioned at the 3′ end of the first coding sequence.

In some aspects, the second rAAV vector genome comprises a second expression cassette comprising a splicing acceptor signal sequence, a second coding sequence that encodes a C-terminal portion of an otoferlin protein (i.e., the entire portion of the otoferlin protein that is not included in the N-terminal portion) positioned at the 3′ end of the splicing acceptor signal sequence (e.g., any of the sizes of a portion of an otoferlin protein described herein and/or any of the C-terminal portions of an otoferlin protein described herein), and a polyadenylation sequence at 3′ end of the second coding sequence (e.g., any of the polyadenylation sequences described herein).

In some aspects, each of the encoded portions is at least 30 amino acid residues in length (e.g., at least 50 amino acids, at least 75 amino acids, or at least 100 amino acids in length), the amino acid sequence of each of the encoded portions does not overlap with the sequence of the other encoded portion, and no single vector of the two different rAAV vectors encodes an active otoferlin protein (e.g., a full-length otoferlin protein). When the two coding sequences of the two rAAV vectors are expressed in a mammalian cell (e.g., any of the mammalian cells described herein), splicing occurs between the splicing donor signal sequence and the splicing acceptor signal sequence, thereby forming a recombined mRNA that encodes an active otoferlin protein (e.g., a full-length otoferlin protein).

In any of the examples of these methods, the amino acid sequence of each of the encoded portions does not overlap with the sequence any of the other encoded portions, and no single vector encodes an active otoferlin protein (e.g., a full-length otoferlin protein).

Each of the two different rAAV vectors includes a coding sequence that encodes a different portion of an otoferlin protein, each of the encoded portions can be at least 30 amino acids (e.g., between about 30 amino acids to about 1600 amino acids, or any of the other subranges of this range described herein).

In some aspects, each of the two different vectors includes a coding sequence that encodes a different portion of an otoferlin protein, each of the encoded portions can encode up to 80% of the amino acid sequence of SEQ ID NO: 5 (e.g., up to 10%, up to 20%, up to 30%, up to 40%, up to 50%, up to 60%, or up to 70% of SEQ ID NO: 5) such that each of the encoded portions is non-overlapping. In some aspects, each of the two different vectors includes a coding sequence that encodes a different portion of an otoferlin protein, each of the encoded portions encoding up to 80% of the amino acid sequence of SEQ ID NO: 5 (e.g., up to 10%, up to 20%, up to 30%, up to 40%, up to 50%, up to 60%, or up to 70% of SEQ ID NO: 5), such that each of the encoded portions is non-overlapping.

Each of the two rAAV vectors may further include an inverted terminal repeat (ITR) to allow head-to-tail recombination. The ITR will be subsequently removed via splicing. For example, the ITR could be a palindromic double-D ITR as described in Yan et al., Proc. Natl. Acad. Sci. U.S.A. 97 (12): 6716-6721, 2000, incorporated in its entirety herein. For example, the ITR could be a AAV serotype-2 ITR as described in Gosh et al., Mol. Ther. 16:124-130, 2008, and Gosh et al., Human Gene Ther. 22:77-83, 2011. Non-limiting examples of splicing acceptor and/or donor signal sequences are known in the art. See, e.g., Reich et al., Human Gene Ther. 14 (1): 37-44, 2003, and Lai et al. (2005) Nat. Biotechnol. 23 (11): 1435-1439, 2005, 2005. The splicing donor and acceptor signal sequences can be any endogenous intron splicing signal of a gene (e.g., an otoferlin gene).

For example, the splicing donor signal sequence
can be:
(SEQ ID NO: 64)
5′-GTAAGTATCAAGGTTACAAGACAGGTTTAAGGAGACCAATAGAAACT
GGGCTTGTCGAGACAGAGAAGACTCTTGCGTTTCT-3′
and the splicing acceptor signal can be:
(SEQ ID NO: 110)
5′-ATAGGCACCTATTGGTCTTACTGACATCCACTTTGCCTTTCTCTCCA
CAG-3′
(see, e.g., Trapani et al., EMBO Mol. Med. 6(2):
194-211,2014).

In some aspects, the splicing donor sequence has at least 70%, at least 75%, at least 80%, at least 85%, at least 90%, at least 91%, at least 92%, at least 93%, at least 94%, at least 95%, at least 96%, at least 97%, at least 98%, at least 90%, or 100% identity to SEQ ID NO: 102. In some aspects, the splicing donor sequence has the sequence of SEQ ID NO: 102.

In some aspects, the splicing acceptor sequence has at least 70%, at least 75%, at least 80%, at least 85%, at least 90%, at least 91%, at least 92%, at least 93%, at least 94%, at least 95%, at least 96%, at least 97%, at least 98%, at least 90%, or 100% identity to SEQ ID NO: 106. In some aspects, the splicing acceptor sequence has the sequence of SEQ ID NO: 106.

Methods of evaluating splicing and splicing efficiency are known in the art (see, e.g., Lai et al., Nat. Biotechnol. 23 (11): 1435-1439, 2005).

### Example 2: Hybrid Vector Trans-Splicing Strategy Using an Alkaline Phosphatase (AP) Highly Recombinogenic Exogenous Gene Region

Two different nucleic acid vectors (e.g., AAV vectors) can also be used in any of the methods described herein to reconstitute an active otoferlin gene (e.g., a full-length otoferlin gene) within a cell following intermolecular i) concatamerization, ii) recombination, iii) trans-splicing, iv) concatamerization and trans-splicing, or v) recombination and trans-splicing. This strategy is a hybrid strategy as it will include concatamerization, homologous recombination and/or trans-splicing. See, e.g., Gosh et al., Mol. Ther. 16:124-130, 2008; Gosh et al., Human Gene Ther. 22:77-83, 2011; and Duan et al., Mol. Ther. 4:383-391, 2001, each incorporated in its entirety herein.

Recombination can occur with a highly recombinogenic DNA sequence that will allow for coding sequence-independent recombination. An non-limiting example of a recombinogenic sequence is an alkaline phosphatase (AP) gene. For example, the recombinogenic sequence can be the middle one-third of the human placental AP complementary DNA, which is 872 bp in length (see, e.g., Gosh et al., 2008). The two different nucleic acid vectors will contain a recombinogenic sequence (e.g., any of the recombinogenic sequences described herein).

Since the hybrid vector will be constructed based on a trans-splicing vector as described in Example 7, an active otoferlin gene (e.g., a full-length otoferlin gene) may be reconstituted using either ITR-mediated recombination and trans-splicing or recombinogenic sequence mediated (e.g., AP-gene mediated) recombination and trans-splicing. After trans-splicing, an active otoferlin gene (e.g., a full-length otoferlin gene) will be reconstituted in the genomic DNA of a mammalian cell (e.g., any mammalian cell described herein).

In some aspects, the first rAAV vector can comprise a first expression cassette comprising a promoter (e.g., any of the promoters described herein), a first coding sequence that encodes an N-terminal portion of an otoferlin protein positioned 3′ of the promoter (e.g., any of the sizes of a portion of an otoferlin protein described herein and/or any of the N-terminal portions of an otoferlin protein described herein), a splicing donor signal sequence positioned at the 3′ end of the first coding sequence, and a first recombinogenic sequence sequence (e.g., an alkaline phosphatase recombinogenic sequence) positioned 3′ of the splicing donor signal sequence.

In some aspects, the second rAAV vector can comprise a second expression cassette comprising a second recombinogenic sequence sequence (e.g., an alkaline phosphatase recombinogenic sequence), a splicing acceptor signal sequence positioned 3′ of the second recombinogenic sequence, a second coding sequence that encodes a C-terminal portion of an otoferlin protein positioned at the 3′ end of the splicing acceptor signal sequence (e.g., any of the sizes of a portion of an otoferlin protein described herein and/or any of the C-terminal portions of an otoferlin protein described herein), and a polyadenylation sequence at 3′ end of the second coding sequence (e.g., any of the polyadenylation sequences described herein).

In some aspects, the first rAAV vector genome can comprise a first expression cassette comprising a promoter (e.g., any of the promoters described herein), a first coding sequence that encodes an N-terminal portion of an otoferlin protein positioned 3′ of the promoter (e.g., any of the sizes of a portion of an otoferlin protein described herein and/or any of the N-terminal portions of an otoferlin protein described herein), a splicing donor signal sequence positioned at the 3′ end of the first coding sequence, and a first recombinogenic sequence sequence (e.g., an alkaline phosphatase recombinogenic sequence) positioned 3′ of the splicing donor signal sequence.

In some aspects, the second rAAV vector genome can comprise a second expression cassette comprising a second recombinogenic sequence sequence (e.g., an alkaline phosphatase recombinogenic sequence), a splicing acceptor signal sequence positioned 3′ of the second recombinogenic sequence, a second coding sequence that encodes a C-terminal portion of an otoferlin protein positioned at the 3′ end of the splicing acceptor signal sequence (e.g., any of the sizes of a portion of an otoferlin protein described herein and/or any of the C-terminal portions of an otoferlin protein described herein), and a polyadenylation sequence at 3′ end of the second coding sequence (e.g., any of the polyadenylation sequences described herein).

In some aspects, each of the encoded portions is at least 30 amino acid residues in length (e.g., at least 50 amino acids, at least 75 amino acids, or at least 100 amino acids in length), the amino acid sequence of each of the encoded portions do not overlap, and no single vector of the two different vectors encodes an active otoferlin protein (e.g., a full-length otoferlin protein). When introduced into a mammalian cell (e.g., any of the mammalian cells described herein) splicing occurs between the splicing donor signal sequence and the splicing acceptor signal sequence, thereby forming an RNA acid that encodes an active otoferlin protein (e.g., a full-length otoferlin protein).

Based on the strategies provided above, one skilled in the art would understand how to develop a strategy using four, five, or six vectors.

The coding sequences provided in the two nucleic acid vectors (e.g., two, will not be overlapping. Each of the two different vectors can include a coding sequence that encodes a different portion of an otoferlin protein, each of the encoded portions being, e.g., at least 30 amino acids (e.g., about 30 amino acids to about 1600 amino acids, or any of the other subranges of this range described herein).

In some aspects, each of the two different rAAV vectors include a coding sequence that encodes a different portion of an otoferlin protein, each of the encoded portions encoding up to 80% of SEQ ID NO: 5 (e.g., up to 10%, up to 20%, up to 30%, up to 40%, up to 50%, up to 60%, up to 70% of SEQ ID NO: 5) such that each of the encoded portions is non-overlapping. In some aspects, each of the two different rAAV vectors include a coding sequence that encodes a different portion of an otoferlin protein, each of the encoded portions encoding up to 80% of SEQ ID NO: 5 (e.g., up to 10%, up to 20%, up to 30%, up to 40%, up to 50%, up to 60%, or up to 70% of SEQ ID NO: 5), such that each of the encoded portions is non-overlapping.

As described in Example 7, each of the two rAAV vectors may further include an inverted terminal repeat (ITR) to allow head-to-tail recombination. The ITR will be subsequently removed via splicing. Examples of ITRs and splicing acceptor and/or donor signal sequences are known in the art and have been described in Example 7.

### Example 3: Hybrid Vector Trans-Splicing Strategy Using a F1 Phage Highly Recombinogenic Exogenous Gene Region (AK)

Two different rAAV vectors can also be used in any of the methods described herein to reconstitute an active otoferlin gene (e.g., a full-length otoferlin gene) within a cell following intermolecular i) concatamerization, ii) recombination, iii) trans-splicing, iv) concatamerization and trans-splicing, or v) recombination and trans-splicing. This strategy is a hybrid strategy as it will include concatamerization, homologous recombination and/or trans-splicing. See, e.g., Trapani et al., EMBO Mol. Med. 6 (2): 194-211, 2014, incorporated in its entirety herein.

As used herein, an F1 phage recombinogenic region (AK) will be used to allow coding sequence-independent recombination. The F1 phage recombinogenic region may be a 77 bp recombinogenic region from the F1 phage genome as described in Trapani et al. (2014) EMBO Mol. Med. 6 (2): 194-211, 2014. The two different rAAV vectors will contain an F1 phage recombinogenic region. Since the hybrid vector will be constructed based on a trans-splicing vector as described in Example 7, a rAAV vector encoding an active otoferlin protein (e.g., a full-length stereocilin protein) may be generated using either ITR-mediated recombination and trans-splicing or F1 phage recombinogenic region-induced recombination and trans-splicing. After trans-splicing, a nucleic acid encoding an active otoferlin protein (e.g., a full-length otoferlin protein) will be generated in a mammalian cell (e.g., any of the mammalian cells described herein).

In some aspects, two rAAV vectors will be used. In some aspects, the first rAAV vector comprises a first expression cassette comprising a promoter (e.g., any of the promoters described herein), a first coding sequence that encodes an N-terminal portion of an otoferlin protein positioned 3′ of the promoter (e.g., any of the sizes of a portion of an otoferlin protein described herein and/or any of the N-terminal portions of an otoferlin protein described herein), a splicing donor signal sequence positioned at the 3′ end of the first coding sequence, and an F1 phage recombinogenic sequence positioned 3′ of the splicing donor signal sequence.

In some aspects, the second rAAV vector comprises a second expression cassette comprising an F1 phage recombinogenic region, a splicing acceptor signal sequence positioned 3′ of the F1 phage recombinogenic region, a second coding sequence that encodes a C-terminal portion of an otoferlin protein positioned at the 3′ end of the splicing acceptor signal sequence (e.g., any of the sizes of a portion of an otoferlin protein described herein and/or any of the C-terminal portions of an otoferlin protein described herein), and a polyadenylation sequence at 3′ end of the second coding sequence (e.g., any of the polyadenylation sequences described herein).

In some aspects, the first rAAV vector genome comprises a first expression cassette comprising a promoter (e.g., any of the promoters described herein), a first coding sequence that encodes an N-terminal portion of an otoferlin protein positioned 3′ of the promoter (e.g., any of the sizes of a portion of an otoferlin protein described herein and/or any of the N-terminal portions of an otoferlin protein described herein), a splicing donor signal sequence positioned at the 3′ end of the first coding sequence, and an F1 phage recombinogenic sequence positioned 3′ of the splicing donor signal sequence.

In some aspects, the second rAAV vector genome comprises a second expression cassette comprising an F1 phage recombinogenic region, a splicing acceptor signal sequence positioned 3′ of the F1 phage recombinogenic region, a second coding sequence that encodes a C-terminal portion of an otoferlin protein positioned at the 3′ end of the splicing acceptor signal sequence (e.g., any of the sizes of a portion of an otoferlin protein described herein and/or any of the C-terminal portions of an otoferlin protein described herein), and a polyadenylation sequence at 3′ end of the second coding sequence (e.g., any of the polyadenylation sequences described herein).

In some aspects, each of the encoded portions is at least 30 amino acid residues in length (e.g., at least 50 amino acids, at least 75 amino acids, or at least 100 amino acids in length), the amino acid sequence of each of the encoded portions do not overlap, and no single vector of the two different rAAV vectors encodes an active otoferlin protein (e.g., a full-length otoferlin protein). When the rAAV vectors are introduced into a mammalian cell (e.g., any of the mammalian cells described herein), splicing occurs between the splicing donor signal sequence and the splicing acceptor signal sequence, thereby forming a recombined nucleic acid that encodes an active otoferlin protein (e.g., a full-length otoferlin protein).

The coding sequences provided in each of the two rAAV vectors will not be overlapping. Each of the two different rAAV vectors include a coding sequence that encodes a different portion of an otoferlin protein, each of the encoded portions being at least 30 amino acids (e.g., about 30 amino acids to about 1600 amino acids, or any of the subranges of this range described herein).

In some aspects, each of the two different rAAV vectors include a coding sequence that encodes a different portion of an otoferlin protein, each of the encoded portions encoding at least one exon and at least one intron of SEQ ID NO: 12 (e.g., at least two exons and at least one intron, at least two exons and at least two introns, at least three exons at least one intron, at least three exons and at least two introns, or at least three exons and at least three introns). In some aspects, each of the two different rAAV vectors includes a coding sequence that encodes a different portion of an otoferlin protein, each of the encoded portions encoding up to 80% of SEQ ID NO: 5 (e.g., up to 10%, up to 20%, up to 30%, up to 40%, up to 50%, up to 60%, or up to 70% of SEQ ID NO: 5) such that each of the encoded portions is non-overlapping. In some aspects, each of the two different rAAV vectors include a coding sequence that encodes a different portion of an otoferlin protein, each of the encoded portions encoding up to 80% of SEQ ID NO: 5 (e.g., up to 10%, up to 20%, up to 30%, up to 40%, up to 50%, up to 60%, or up to 70% of SEQ ID NO: 5), such that each of the encoded portions is non-overlapping.

As described in Example 7 each of the at least two nucleic acid vectors may further include an inverted terminal repeat (ITR) to allow head-to-tail recombination. The ITR will be subsequently removed via splicing. Examples of ITRs and splicing acceptor and/or donor signals are known in the art and have been described in Example 7.

### Example 4: In Vitro Expression of Full-Length Human Otoferlin Using Two Vectors

As in other dual vector approaches, two transgenes, each comprising a portion of the full-length transcript, are packaged in separate vectors and provided, together, to contact a target, e.g., a target cell population in, e.g., a subject in need thereof. The present example provides a set of vectors which were generated to each include a nucleic acid sequence that comprises a portion of the coding sequence of the human otoferlin (OTOF) gene or OTOF cDNA.

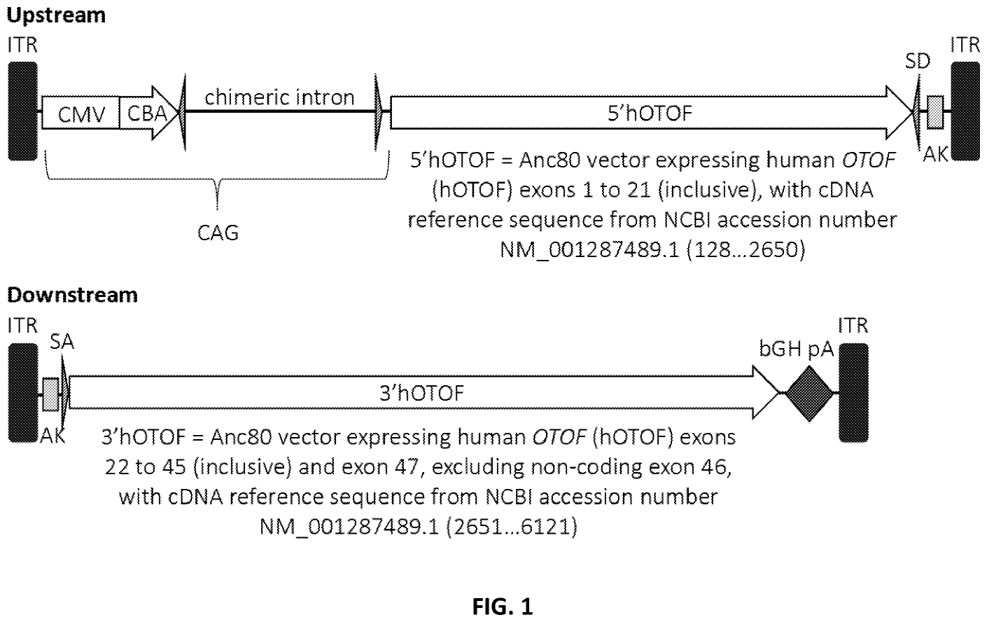

AAVAnc80-hOTOF is comprised of two recombinant vectors (AAVAnc80-5′hOTOF and AAVAnc80-3′hOTOF) containing single-stranded DNA genomes of 4452 and 3905 nucleotides (exclusive of ITRs), respectively (FIG. 1). The single-stranded DNA genomes are encapsidated by the AAVAnc80 (also referred to as Anc80L65; Zinn 2015) capsid.

The upstream DNA genome (AAVAnc80-5′hOTOF) includes a eukaryotic expression cassette encoding the following promoter and regulatory sequences: the cytomegalovirus (CMV) early enhancer element (SEQ ID NO: 98); the chicken beta actin (CBA) gene sequence located between 5′ flanking region and the proximal region of the second exon (SEQ ID NO: 99); and 3′ splice sequence derived from the rabbit beta globin (RBG) gene (SEQ ID NO: 100). This is commonly referred to as the CAG promoter (Miyazaki 1989; Niwa 1991; Orbán 2009). Following this hybrid regulatory element are: the human OTOF (hOTOF) coding sequence, exons 1 through 21 (inclusive) (SEQ ID NO: 101); a synthetic splice donor (SD) to facilitate trans-splicing (Trapani 2014) (SEQ ID NO: 102); and a 77-base pair (bp) AK recombinogenic sequence (Trapani 2014; Trapani 2015) (SEQ ID NO: 103). The full-length AAVAnc80-5′hOTOF sequence has the sequence of SEQ ID NO: 96.

The downstream DNA genome (AAVAnc80-3′hOTOF) includes a eukaryotic expression cassette encoding the following: the same 77-bp AK recombinogenic sequence (Trapani 2014; Trapani 2015) (SEQ ID NO: 103); a synthetic splice acceptor (SA) to facilitate trans-splicing (Trapani 2014) (SEQ ID NO: 106); the human OTOF (hOTOF) coding sequence, exons 22 through 45 (inclusive) and exon 47, excluding non-coding exon 46 (SEQ ID NO: 107); and the bovine growth hormone (bGH) polyadenylation (pA) signal (SEQ ID NO: 108). Each expression cassette is flanked by AAV2 inverted terminal repeats (ITRs) (SEQ ID NOs: 97 and 104, respectively). The full-length AAVAnc80-3′hOTOF sequence has the sequence of SEQ ID NO: 105.

A target cell needs to receive a copy of both the upstream and the downstream transgene and, based on the dual vector design, these transgenes recombine at the DNA level and lead to generation of a full-length mRNA transcript (McClements 2017), specifically human OTOF based on isoform 5, according to NCBI accession number NM_001287489.1 (128 . . . 6121).

In some aspects, when each of the upstream and downstream vectors is administered to a subject in need thereof, the constructs concatemerize within a given cell. In some aspects, concatemerized full-length OTOF is expressed and generates functional otoferlin protein.

In some aspects, when each of the upstream and downstream vectors is administered to a subject in need thereof, the constructs recombine within a given cell. In some aspects, recombined full-length OTOF is expressed and generates functional otoferlin protein.

Pairs of these vectors are used to treat a human subject suffering from or susceptible to hearing loss. A composition comprising both vectors of the dual AAV vector system, AAVAnc80.AKhOTOF5 and AAVAnc80.AKhOTOF3, is introduced into at least one cochlea of the human subject. Hearing function is tested in the human subject at days 15, 30, 45, 60 and 90 after administration, and is compared to the functional hearing of the human subject prior to receiving treatment or to a human subject that did not receive treatment.

AAVAnc80-5′hOTOF The AAVAnc80-5′hOTOF construct comprises two ITRs (SEQ ID NO: 97 and 104), a CAG promoter (identified by SEQ ID NOs: 98, 99, and 100, comprising a CMV early enhancer element (SEQ ID NO: 98), chicken beta actin gene sequence (SEQ ID NO: 99), and a chimeric intron comprising 3′ splice sequence from the rabbit beta globin gene (SEQ ID NO: 100)), a 5′OTOF coding region (SEQ ID NO: 101), a SD intron sequence (SEQ ID NO: 102), and an AK recombinogenic sequence (SEQ ID NO: 103). The full-length AAVAnc80-5′hOTOF is represented by SEQ ID NO: 96.

TABLE 1
AAVAnc80-5′hOTOF

| | | SEQ ID |
|---|---|---|
| Name | SEQUENCE | NO |
| AAVAnc80- | TTGGCCACTCCCTCTCTGCGCGCTCGCTCGCTCACTGAGGCCGCC | 96 |
| 5′hOTOF | CGGGCAAAGCCCGGGCGTCGGGCGACCTTTGGTCGCCCGGCCTCA | |
| | GTGAGCGAGCGAGCGCGCAGAGAGGGAGTGGCCAACTCCATCACT | |
| | AGGGGTTCCTTTGTCGACGCGGCCGCACGCGTGACATTGATTATT | |
| | GACTAGTTATTAATAGTAATCAATTACGGGGTCATTAGTTCATAG | |
| | CCCATATATGGAGTTCCGCGTTACATAACTTACGGTAAATGGCCC | |
| | GCCTGGCTGACCGCCCAACGACCCCCGCCCATTGACGTCAATAAT | |
| | GACGTATGTTCCCATAGTAACGCCAATAGGGACTTTCCATTGACG | |
| | TCAATGGGTGGACTATTTACGGTAAACTGCCCACTTGGCAGTACA | |
| | TCAAGTGTATCATATGCCAAGTACGCCCCCTATTGACGTCAATGA | |
| | CGGTAAATGGCCCGCCTGGCATTATGCCCAGTACATGACCTTATG | |
| | GGACTTTCCTACTTGGCAGTACATCTACGTATTAGTCATCGCTAT | |
| | TACCATGGGTCGAGGTGAGCCCCACGTTCTGCTTCACTCTCCCCA | |
| | TCTCCCCCCCCTCCCCACCCCCAATTTTGTATTTATTTATTTTTT | |
| | AATTATTTTGTGCAGCGATGGGGGGGGGGGGGGGGGGGGGGGGCG | |
| | CGCCAGGCGGGGCGGGGGGGGGCGAGGGGCGGGGCGGGGCGAGGC | |
| | GGAGAGGTGCGGCGGCAGCCAATCAGAGCGGCGCGCTCCGAAAGT | |
| | TTCCTTTTATGGCGAGGCGGCGGCGGCGGCGGCCCTATAAAAAGC | |
| | GAAGCGCGCGGCGGGGGGGAGTCGCTGCGTTGCCTTCGCCCCGTG | |
| | CCCCGCTCCGCGCCGCCTCGCGCCGCCCGCCCCGGCTCTGACTGA | |
| | CCGCGTTACTCCCACAGGTGAGCGGGCGGGACGGCCCTTCTCCTC | |
| | CGGGCTGTAATTAGCGCTTGGTTTAATGACGGCTCGTTTCTTTTC | |
| | TGTGGCTGCGTGAAAGCCTTAAAGGGCTCCGGGAGGGCCCTTTGT | |
| | GCGGGGGGGAGCGGCTCGGGGGGTGCGTGCGTGTGTGTGTGCGTG | |
| | GGGAGCGCCGCGTGCGGCCCGCGCTGCCCGGCGGCTGTGAGCGCT | |
| | GCGGGCGCGGCGCGGGGCTTTGTGCGCTCCGCGTGTGCGCGAGGG | |
| | GAGCGCGGCCGGGGGCGGTGCCCCGCGGTGCGGGGGGGCTGCGAG | |
| | GGGAACAAAGGCTGCGTGCGGGGTGTGTGCGTGGGGGGGTGAGCA | |
| | GGGGGTGTGGGCGCGGCGGTCGGGCTGTAACCCCCCCCTGCACCC | |
| | CCCTCCCCGAGTTGCTGAGCACGGCCCGGCTTCGGGTGCGGGGCT | |
| | CCGTGCGGGGCGTGGCGCGGGGCTCGCCGTGCCGGGCGGGGGGTG | |
| | GCGGCAGGTGGGGGTGCCGGGGGGGGGGGGGCCGCCTCGGGCCGG | |
| | GGAGGGCTCGGGGGAGGGGCGCGGCGGCCCCCGGAGCGCCGGCGG | |
| | CTGTCGAGGCGCGGCGAGCCGCAGCCATTGCCTTTTATGGTAATC | |
| | GTGCGAGAGGGCGCAGGGACTTCCTTTGTCCCAAATCTGTGCGGA | |
| | GCCGAAATCTGGGAGGCGCCGCCGCACCCCCTCTAGCGGGCGCGG | |
| | GGCGAAGCGGTGCGGCGCCGGCAGGAAGGAAATGGGGGGGGAGGG | |
| | CCTTCGTGCGTCGCCGCGCCGCCGTCCCCTTCTCCCTCTCCAGCC | |
| | TCGGGGCTGTCCGCGGGGGGACGGCTGCCTTCGGGGGGGACGGGG | |
| | CAGGGGGGGGTTCGGCTTCTGGCGTGTGACCGGCGGCTCTAGAGC | |
| | CTCTGCTAACCATGTTCATGCCTTCTTCTTTTTCCTACAGCTCCT | |
| | GGGCAACGTGCTGGTTATTGTGACCGGTGCCACCATGGCCTTGCT | |
| | CATCCACCTCAAGACAGTCTCGGAGCTGCGGGGCAGGGGCGACCG | |
| | GATCGCCAAAGTGACTTTCCGAGGGCAATCCTTCTACTCTCGGGT | |
| | CCTGGAGAACTGTGAGGATGTGGCTGACTTTGATGAGACATTTCG | |
| | GTGGCCGGTGGCCAGCAGCATCGACAGAAATGAGATGCTGGAGAT | |
| | TCAGGTTTTCAACTACAGCAAAGTCTTCAGCAACAAGCTCATCGG | |
| | GACCTTCCGCATGGTGCTGCAGAAGGTGGTAGAGGAGAGCCATGT | |
| | GGAGGTGACTGACACGCTGATTGATGACAACAATGCTATCATCAA | |
| | GACCAGCCTGTGCGTGGAGGTCCGGTATCAGGCCACTGACGGCAC | |
| | AGTGGGCTCCTGGGACGATGGGGACTTCCTGGGAGATGAGTCTCT | |
| | TCAAGAGGAAGAGAAGGACAGCCAAGAGACGGATGGACTGCTCCC | |
| | AGGCTCCCGGCCCAGCTCCCGGCCCCCAGGAGAGAAGAGCTTCCG | |
| | GAGAGCCGGGAGGAGCGTGTTCTCCGCCATGAAGCTCGGCAAAAA | |
| | CAGGTCTCACAAGGAGGAGCCCCAAAGACCAGATGAACCGGCGGT | |
| | GCTGGAGATGGAAGACCTTGACCATCTGGCCATTCGGCTAGGAGA | |
| | TGGACTGGATCCCGACTCGGTGTCTCTAGCCTCAGTCACAGCTCT | |
| | CACCACTAATGTCTCCAACAAGCGATCTAAGCCAGACATTAAGAT | |
| | GGAGCCAAGTGCTGGGCGGCCCATGGATTACCAGGTCAGCATCAC | |
| | GGTGATCGAGGCCCGGCAGCTGGTGGGCTTGAACATGGACCCTGT | |
| | GGTGTGCGTGGAGGTGGGTGACGACAAGAAGTACACATCCATGAA | |
| | GGAGTCCACTAACTGCCCCTATTACAACGAGTACTTCGTCTTCGA | |
| | CTTCCATGTCTCTCCGGATGTCATGTTTGACAAGATCATCAAGAT | |
| | TTCGGTGATTCACTCCAAGAACCTGCTGCGCAGTGGCACCCTGGT | |
| | GGGCTCCTTCAAAATGGACGTGGGAACCGTGTACTCGCAGCCAGA | |
| | GCACCAGTTCCATCACAAGTGGGCCATCCTGTCTGACCCCGATGA | |
| | CATCTCCTCGGGGCTGAAGGGCTACGTGAAGTGTGACGTTGCCGT | |
| | GGTGGGCAAAGGGGACAACATCAAGACGCCCCACAAGGCCAATGA | |
| | GACCGACGAAGATGACATTGAGGGGAACTTGCTGCTCCCCGAGGG | |
| | GGTGCCCCCCGAACGCCAGTGGGCCCGGTTCTATGTGAAAATTTA | |
| | CCGAGCAGAGGGGCTGCCCCGTATGAACACAAGCCTCATGGCCAA | |
| | TGTAAAGAAGGCTTTCATCGGTGAAAACAAGGACCTCGTGGACCC | |
| | GCAGAAGAGCAGCTATGAGCCCCTGTGGAATGAGCAGGTCGTCTT | |
| | TACAGACCTCTTCCCCCCACTCTGCAAACGCATGAAGGTGCAGAT | |
| | CCGAGACTCGGACAAGGTCAACGACGTGGCCATCGGCACCCACTT | |
| | CATTGACCTGCGCAAGATTTCTAATGACGGAGACAAAGGCTTCCT | |
| | GCCCACACTGGGCCCAGCCTGGGTGAACATGTACGGCTCCACACG | |
| | TAACTACACGCTGCTGGATGAGCATCAGGACCTGAACGAGGGCCT | |
| | GGGGGAGGGTGTGTCCTTCCGGGCCCGGCTCCTGCTGGGCCTGGC | |
| | TGTGGAGATCGTAGACACCTCCAACCCTGAGCTCACCAGCTCCAC | |
| | AGAGGTGCAGGTGGAGCAGGCCACGCCCATCTCGGAGAGCTGTGC | |
| | AGGTAAAATGGAAGAATTCTTTCTCTTTGGAGCCTTCCTGGAGGC | |
| | CTCAATGATCGACCGGAGAAACGGAGACAAGCCCATCACCTTTGA | |
| | GGTCACCATAGGCAACTATGGGAACGAAGTTGATGGCCTGTCCCG | |
| | GCCCCAGCGGCCTCGGCCCCGGAAGGAGCCGGGGGATGAGGAAGA | |
| | AGTAGACCTGATTCAGAACGCAAGTGATGACGAGGCCGGTGATGC | |
| | CGGGGACCTGGCCTCAGTCTCCTCCACTCCACCAATGCGGCCCCA | |
| | GGTCACCGACAGGAACTACTTCCATCTGCCCTACCTGGAGCGAAA | |
| | GCCCTGCATCTACATCAAGAGCTGGTGGCCGGACCAGCGCCGCCG | |
| | CCTCTACAATGCCAACATCATGGACCACATTGCCGACAAGCTGGA | |
| | AGAAGGCCTGAACGACATACAGGAGATGATCAAAACGGAGAAGTC | |
| | CTACCCTGAGCGTCGCCTGCGGGGCGTCCTGGAGGAGCTGAGCTG | |
| | TGGCTGCTGCCGCTTCCTCTCCCTCGCTGACAAGGACCAGGGCCA | |
| | CTCATCCCGCACCAGGCTTGACCGGGAGCGCCTCAAGTCCTGCAT | |
| | GAGGGAGCTGGAAAACATGGGGCAGCAGGCCAGGATGCTGCGGGC | |
| | CCAGGTGAAGCGGCACACGGTGCGGGACAAGCTGAGGCTGTGCCA | |
| | GAACTTCCTGCAGAAGCTGCGCTTCCTGGCGGACGAGGTAAGTAT | |
| | CAAGGTTACAAGACAGGTTTAAGGAGACCAATAGAAACTGGGCTT | |
| | GTCGAGACAGAGAAGACTCTTGCGTTTCTGGGATTTTGCCGATTT | |
| | CGGCCTATTGGTTAAAAAATGAGCTGATTTAACAAAAATTTAACG | |
| | CGAATTTTAACAAAATAAGCTTGAATTCAGCTGACGTGCCTCGGA | |
| | CCGCCTAGGAGGAACCCCTAGTGATGGAGTTGGCCACTCCCTCTC | |
| | TGCGCGCTCGCTCGCTCACTGAGGCCGGGCGACCAAAGGTCGCCC | |
| | GACGCCCGGGCTTTGCCCGGGCGGCCTCAGTGAGCGAGCGAGCGC | |
| | GCAGAGAGGGAGTGGCCAA | |
| 5′ITR | TTGGCCACTCCCTCTCTGCGCGCTCGCTCGCTCACTGAGGCCGCC | 97 |
| | CGGGCAAAGCCCGGGCGTCGGGCGACCTTTGGTCGCCCGGCCTCA | |
| | GTGAGCGAGCGAGCGCGCAGAGAGGGAGTGGCCAACTCCATCACT | |
| | AGGGGTTCCT | |
| CMV | GACATTGATTATTGACTAGTTATTAATAGTAATCAATTACGGGGT | 98 |
| enhancer | CATTAGTTCATAGCCCATATATGGAGTTCCGCGTTACATAACTTA | |
| | CGGTAAATGGCCCGCCTGGCTGACCGCCCAACGACCCCCGCCCAT | |
| | TGACGTCAATAATGACGTATGTTCCCATAGTAACGCCAATAGGGA | |
| | CTTTCCATTGACGTCAATGGGTGGACTATTTACGGTAAACTGCCC | |
| | ACTTGGCAGTACATCAAGTGTATCATATGCCAAGTACGCCCCCTA | |
| | TTGACGTCAATGACGGTAAATGGCCCGCCTGGCATTATGCCCAGT | |
| | ACATGACCTTATGGGACTTTCCTACTTGGCAGTACATCTACGTAT | |
| | TAGTCATCGCTATTACCATGG | |
| CBA gene | GTCGAGGTGAGCCCCACGTTCTGCTTCACTCTCCCCATCTCCCCC | 99 |
| sequence | CCCTCCCCACCCCCAATTTTGTATTTATTTATTTTTTAATTATTT | |
| | TGTGCAGCGATGGGGGGGGGGGGGGGGGGGGGGGGCGCGCCAGGC | |
| | GGGGGGGGGCGGGGCGAGGGGCGGGGCGGGGCGAGGCGGAGAGGT | |
| | GCGGCGGCAGCCAATCAGAGCGGCGCGCTCCGAAAGTTTCCTTTT | |
| | ATGGCGAGGCGGCGGCGGCGGCGGCCCTATAAAAAGCGAAGCGCG | |
| | CGGCGGGCG | |
| Chimeric | GGAGTCGCTGCGTTGCCTTCGCCCCGTGCCCCGCTCCGCGCCGCC | 100 |
| intron | TCGCGCCGCCCGCCCCGGCTCTGACTGACCGCGTTACTCCCACAG | |
| | GTGAGCGGGCGGGACGGCCCTTCTCCTCCGGGCTGTAATTAGCGC | |
| | TTGGTTTAATGACGGCTCGTTTCTTTTCTGTGGCTGCGTGAAAGC | |
| | CTTAAAGGGCTCCGGGAGGGCCCTTTGTGCGGGGGGGAGCGGCTC | |
| | GGGGGGTGCGTGCGTGTGTGTGTGCGTGGGGAGCGCCGCGTGCGG | |
| | CCCGCGCTGCCCGGCGGCTGTGAGCGCTGCGGGCGCGGCGCGGGG | |
| | CTTTGTGCGCTCCGCGTGTGCGCGAGGGGAGCGCGGCCGGGGGCG | |
| | GTGCCCCGCGGTGCGGGGGGGCTGCGAGGGGAACAAAGGCTGCGT | |
| | GCGGGGTGTGTGCGTGGGGGGGTGAGCAGGGGGTGTGGGCGCGGC | |
| | GGTCGGGCTGTAACCCCCCCCTGCACCCCCCTCCCCGAGTTGCTG | |
| | AGCACGGCCCGGCTTCGGGTGCGGGGCTCCGTGCGGGGCGTGGCG | |
| | CGGGGCTCGCCGTGCCGGGGGGGGGGTGGCGGCAGGTGGGGGTGC | |
| | CGGGGGGGGGGGGGCCGCCTCGGGCCGGGGAGGGCTCGGGGGAGG | |
| | GGCGCGGCGGCCCCCGGAGCGCCGGCGGCTGTCGAGGCGCGGCGA | |
| | GCCGCAGCCATTGCCTTTTATGGTAATCGTGCGAGAGGGCGCAGG | |
| | GACTTCCTTTGTCCCAAATCTGTGCGGAGCCGAAATCTGGGAGGC | |
| | GCCGCCGCACCCCCTCTAGCGGGCGCGGGGCGAAGCGGTGCGGCG | |
| | CCGGCAGGAAGGAAATGGGCGGGGAGGGCCTTCGTGCGTCGCCGC | |
| | GCCGCCGTCCCCTTCTCCCTCTCCAGCCTCGGGGCTGTCCGCGGG | |
| | GGGACGGCTGCCTTCGGGGGGGACGGGGCAGGGGGGGGTTCGGCT | |
| | TCTGGCGTGTGACCGGCGGCTCTAGAGCCTCTGCTAACCATGTTC | |
| | ATGCCTTCTTCTTTTTCCTACAG | |
| 5′OTOF | ATGGCCTTGCTCATCCACCTCAAGACAGTCTCGGAGCTGCGGGGC | 101 |
| | AGGGGCGACCGGATCGCCAAAGTGACTTTCCGAGGGCAATCCTTC | |
| | TACTCTCGGGTCCTGGAGAACTGTGAGGATGTGGCTGACTTTGAT | |
| | GAGACATTTCGGTGGCCGGTGGCCAGCAGCATCGACAGAAATGAG | |
| | ATGCTGGAGATTCAGGTTTTCAACTACAGCAAAGTCTTCAGCAAC | |
| | AAGCTCATCGGGACCTTCCGCATGGTGCTGCAGAAGGTGGTAGAG | |
| | GAGAGCCATGTGGAGGTGACTGACACGCTGATTGATGACAACAAT | |
| | GCTATCATCAAGACCAGCCTGTGCGTGGAGGTCCGGTATCAGGCC | |
| | ACTGACGGCACAGTGGGCTCCTGGGACGATGGGGACTTCCTGGGA | |
| | GATGAGTCTCTTCAAGAGGAAGAGAAGGACAGCCAAGAGACGGAT | |
| | GGACTGCTCCCAGGCTCCCGGCCCAGCTCCCGGCCCCCAGGAGAG | |
| | AAGAGCTTCCGGAGAGCCGGGAGGAGCGTGTTCTCCGCCATGAAG | |
| | CTCGGCAAAAACAGGTCTCACAAGGAGGAGCCCCAAAGACCAGAT | |
| | GAACCGGCGGTGCTGGAGATGGAAGACCTTGACCATCTGGCCATT | |
| | CGGCTAGGAGATGGACTGGATCCCGACTCGGTGTCTCTAGCCTCA | |
| | GTCACAGCTCTCACCACTAATGTCTCCAACAAGCGATCTAAGCCA | |
| | GACATTAAGATGGAGCCAAGTGCTGGGCGGCCCATGGATTACCAG | |
| | GTCAGCATCACGGTGATCGAGGCCCGGCAGCTGGTGGGCTTGAAC | |
| | ATGGACCCTGTGGTGTGCGTGGAGGTGGGTGACGACAAGAAGTAC | |
| | ACATCCATGAAGGAGTCCACTAACTGCCCCTATTACAACGAGTAC | |
| | TTCGTCTTCGACTTCCATGTCTCTCCGGATGTCATGTTTGACAAG | |
| | ATCATCAAGATTTCGGTGATTCACTCCAAGAACCTGCTGCGCAGT | |
| | GGCACCCTGGTGGGCTCCTTCAAAATGGACGTGGGAACCGTGTAC | |
| | TCGCAGCCAGAGCACCAGTTCCATCACAAGTGGGCCATCCTGTCT | |
| | GACCCCGATGACATCTCCTCGGGGCTGAAGGGCTACGTGAAGTGT | |
| | GACGTTGCCGTGGTGGGCAAAGGGGACAACATCAAGACGCCCCAC | |
| | AAGGCCAATGAGACCGACGAAGATGACATTGAGGGGAACTTGCTG | |
| | CTCCCCGAGGGGGTGCCCCCCGAACGCCAGTGGGCCCGGTTCTAT | |
| | GTGAAAATTTACCGAGCAGAGGGGCTGCCCCGTATGAACACAAGC | |
| | CTCATGGCCAATGTAAAGAAGGCTTTCATCGGTGAAAACAAGGAC | |
| | CTCGTGGACCCCTACGTGCAAGTCTTCTTTGCTGGCCAGAAGGGC | |
| | AAGACTTCAGTGCAGAAGAGCAGCTATGAGCCCCTGTGGAATGAG | |
| | CAGGTCGTCTTTACAGACCTCTTCCCCCCACTCTGCAAACGCATG | |
| | AAGGTGCAGATCCGAGACTCGGACAAGGTCAACGACGTGGCCATC | |
| | GGCACCCACTTCATTGACCTGCGCAAGATTTCTAATGACGGAGAC | |
| | AAAGGCTTCCTGCCCACACTGGGCCCAGCCTGGGTGAACATGTAC | |
| | GGCTCCACACGTAACTACACGCTGCTGGATGAGCATCAGGACCTG | |
| | AACGAGGGCCTGGGGGAGGGTGTGTCCTTCCGGGCCCGGCTCCTG | |
| | CTGGGCCTGGCTGTGGAGATCGTAGACACCTCCAACCCTGAGCTC | |
| | ACCAGCTCCACAGAGGTGCAGGTGGAGCAGGCCACGCCCATCTCG | |
| | GAGAGCTGTGCAGGTAAAATGGAAGAATTCTTTCTCTTTGGAGCC | |
| | TTCCTGGAGGCCTCAATGATCGACCGGAGAAACGGAGACAAGCCC | |
| | ATCACCTTTGAGGTCACCATAGGCAACTATGGGAACGAAGTTGAT | |
| | GGCCTGTCCCGGCCCCAGCGGCCTCGGCCCCGGAAGGAGCCGGGG | |
| | GATGAGGAAGAAGTAGACCTGATTCAGAACGCAAGTGATGACGAG | |
| | GCCGGTGATGCCGGGGACCTGGCCTCAGTCTCCTCCACTCCACCA | |
| | ATGCGGCCCCAGGTCACCGACAGGAACTACTTCCATCTGCCCTAC | |
| | CTGGAGCGAAAGCCCTGCATCTACATCAAGAGCTGGTGGCCGGAC | |
| | CAGCGCCGCCGCCTCTACAATGCCAACATCATGGACCACATTGCC | |
| | GACAAGCTGGAAGAAGGCCTGAACGACATACAGGAGATGATCAAA | |
| | ACGGAGAAGTCCTACCCTGAGCGTCGCCTGCGGGGCGTCCTGGAG | |
| | GAGCTGAGCTGTGGCTGCTGCCGCTTCCTCTCCCTCGCTGACAAG | |
| | GACCAGGGCCACTCATCCCGCACCAGGCTTGACCGGGAGCGCCTC | |
| | AAGTCCTGCATGAGGGAGCTGGAAAACATGGGGCAGCAGGCCAGG | |
| | ATGCTGCGGGCCCAGGTGAAGCGGCACACGGTGCGGGACAAGCTG | |
| | AGGCTGTGCCAGAACTTCCTGCAGAAGCTGCGCTTCCTGGCGGAC | |
| | GAG | |
| SD intron | GTAAGTATCAAGGTTACAAGACAGGTTTAAGGAGACCAATAGAAA | 102 |
| | CTGGGCTTGTCGAGACAGAGAAGACTCTTGCGTTTCT | |
| AK | GGGATTTTGCCGATTTCGGCCTATTGGTTAAAAAATGAGCTGATT | 103 |
| | TAACAAAAATTTAACGCGAATTTTAACAAAAT | |
| 3′ITR | AGGAACCCCTAGTGATGGAGTTGGCCACTCCCTCTCTGCGCGCTC | 104 |
| | GCTCGCTCACTGAGGCCGGGCGACCAAAGGTCGCCCGACGCCCGG | |
| | GCTTTGCCCGGGCGGCCTCAGTGAGCGAGCGAGCGCGCAGAGAGG | |
| | GAGTGGCCAA | |

AAVAnc80-3′% OTOF The AAVAnc80-3′hOTOF construct comprises two ITRs (SEQ ID NOS: 97 and 104), an AK recombinogenic sequence (SEQ ID NO: 103), an SA intron sequence (SEQ ID NO:106), a 3′OTOF coding region (SEQ ID NO: 107), and a bgH polyA sequence (SEQ ID NO: 108). The full-length AAVAnc80-3′hOTOF is represented by SEQ ID NO: 105.

TABLE 2
AAVAnc80-3′hOTOF

| | | SEQ ID |
|---|---|---|
| Name | SEQUENCE | NO |
| AAVAnc80- | TTGGCCACTCCCTCTCTGCGCGCTCGCTCGCTCACTGAGGCCGCC | 105 |
| 3′hOTOF | CGGGCAAAGCCCGGGCGTCGGGCGACCTTTGGTCGCCCGGCCTCA | |
| | GTGAGCGAGCGAGCGCGCAGAGAGGGAGTGGCCAACTCCATCACT | |
| | AGGGGTTCCTTTGTCGACGCGGCCGCACGCGTGGGATTTTGCCGA | |
| | TTTCGGCCTATTGGTTAAAAAATGAGCTGATTTAACAAAAATTTA | |
| | ACGCGAATTTTAACAAAATGATAGGCACCTATTGGTCTTACTGAC | |
| | ATCCACTTTGCCTTTCTCTCCACAGCCCCAGCACAGCATTCCCGA | |
| | CATCTTCATCTGGATGATGAGCAACAACAAGCGTGTCGCCTATGC | |
| | CCGTGTGCCCTCCAAGGACCTGCTCTTCTCCATCGTGGAGGAGGA | |
| | GACTGGCAAGGACTGCGCCAAGGTCAAGACGCTCTTCCTTAAGCT | |
| | GCCAGGGAAGCGGGGCTTCGGCTCGGCAGGCTGGACAGTGCAGGC | |
| | CAAGGTGGAGCTGTACCTGTGGCTGGGCCTCAGCAAACAGCGCAA | |
| | GGAGTTCCTGTGCGGCCTGCCCTGTGGCTTCCAGGAGGTCAAGGC | |
| | AGCCCAGGGCCTGGGCCTGCATGCCTTCCCACCCGTCAGCCTGGT | |
| | CTACACCAAGAAGCAGGCGTTCCAGCTCCGAGCGCACATGTACCA | |
| | GGCCCGCAGCCTCTTTGCCGCCGACAGCAGCGGACTCTCAGACCC | |
| | CTTTGCCCGCGTCTTCTTCATCAATCAGAGTCAGTGCACAGAGGT | |
| | GCTGAATGAGACCCTGTGTCCCACCTGGGACCAGATGCTGGTGTT | |
| | CGACAACCTGGAGCTCTATGGTGAAGCTCATGAGCTGAGGGACGA | |
| | TCCGCCCATCATTGTCATTGAAATCTATGACCAGGATTCCATGGG | |
| | CAAAGCTGACTTCATGGGCCGGACCTTCGCCAAACCCCTGGTGAA | |
| | GATGGCAGACGAGGCGTACTGCCCACCCCGCTTCCCACCTCAGCT | |
| | CGAGTACTACCAGATCTACCGTGGCAACGCCACAGCTGGAGACCT | |
| | GCTGGCGGCCTTCGAGCTGCTGCAGATTGGACCAGCAGGGAAGGC | |
| | TGACCTGCCCCCCATCAATGGCCCGGTGGACGTGGACCGAGGTCC | |
| | CATCATGCCCGTGCCCATGGGCATCCGGCCCGTGCTCAGCAAGTA | |
| | CCGAGTGGAGGTGCTGTTCTGGGGCCTACGGGACCTAAAGCGGGT | |
| | GAACCTGGCCCAGGTGGACCGGCCACGGGTGGACATCGAGTGTGC | |
| | AGGGAAGGGGGTGCAGTCGTCCCTGATCCACAATTATAAGAAGAA | |
| | CCCCAACTTCAACACCCTCGTCAAGTGGTTTGAAGTGGACCTCCC | |
| | AGAGAACGAGCTGCTGCACCCGCCCTTGAACATCCGTGTGGTGGA | |
| | CTGCCGGGCCTTCGGTCGCTACACACTGGTGGGCTCCCATGCCGT | |
| | CAGCTCCCTGCGACGCTTCATCTACCGGCCCCCAGACCGCTCGGC | |
| | CCCCAGCTGGAACACCACGGTCAGGCTTCTCCGGCGCTGCCGTGT | |
| | GCTGTGCAATGGGGGCTCCTCCTCTCACTCCACAGGGGAGGTTGT | |
| | GGTGACTATGGAGCCAGAGGTACCCATCAAGAAACTGGAGACCAT | |
| | GGTGAAGCTGGACGCGACTTCTGAAGCTGTTGTCAAGGTGGATGT | |
| | GGCTGAGGAGGAGAAGGAGAAGAAGAAGAAGAAGAAGGGCACTGC | |
| | GGAGGAGCCAGAGGAGGAGGAGCCAGACGAGAGCATGCTGGACTG | |
| | GTGGTCCAAGTACTTTGCCTCCATTGACACCATGAAGGAGCAACT | |
| | TCGACAACAAGAGCCCTCTGGAATTGACTTGGAGGAGAAGGAGGA | |
| | AGTGGACAATACCGAGGGCCTGAAGGGGTCAATGAAGGGCAAGGA | |
| | GAAGGCAAGGGCTGCCAAAGAGGAGAAGAAGAAGAAAACTCAGAG | |
| | CTCTGGCTCTGGCCAGGGGTCCGAGGCCCCCGAGAAGAAGAAACC | |
| | CAAGATTGATGAGCTTAAGGTATACCCCAAAGAGCTGGAGTCCGA | |
| | GTTTGATAACTTTGAGGACTGGCTGCACACTTTCAACTTGCTTCG | |
| | GGGCAAGACCGGGGATGATGAGGATGGCTCCACCGAGGAGGAGCG | |
| | CATTGTGGGACGCTTCAAGGGCTCCCTCTGCGTGTACAAAGTGCC | |
| | ACTCCCAGAGGACGTGTCCCGGGAAGCCGGCTACGACTCCACCTA | |
| | CGGCATGTTCCAGGGCATCCCGAGCAATGACCCCATCAATGTGCT | |
| | GGTCCGAGTCTATGTGGTCCGGGCCACGGACCTGCACCCTGCTGA | |
| | CATCAACGGCAAAGCTGACCCCTACATCGCCATCCGGCTAGGCAA | |
| | GACTGACATCCGCGACAAGGAGAACTACATCTCCAAGCAGCTCAA | |
| | CCCTGTCTTTGGGAAGTCCTTTGACATCGAGGCCTCCTTCCCCAT | |
| | GGAATCCATGCTGACGGTGGCTGTGTATGACTGGGACCTGGTGGG | |
| | CACTGATGACCTCATTGGGGAAACCAAGATCGACCTGGAGAACCG | |
| | CTTCTACAGCAAGCACCGCGCCACCTGCGGCATCGCCCAGACCTA | |
| | CTCCACACATGGCTACAATATCTGGCGGGACCCCATGAAGCCCAG | |
| | CCAGATCCTGACCCGCCTCTGCAAAGACGGCAAAGTGGACGGCCC | |
| | CCACTTTGGGCCCCCTGGGAGAGTGAAGGTGGCCAACCGCGTCTT | |
| | CACTGGGCCCTCTGAGATTGAGGACGAGAACGGTCAGAGGAAGCC | |
| | CACAGACGAGCATGTGGCGCTGTTGGCCCTGAGGCACTGGGAGGA | |
| | CATCCCCCGCGCAGGCTGCCGCCTGGTGCCAGAGCATGTGGAGAC | |
| | GAGGCCGCTGCTCAACCCCGACAAGCCGGGCATCGAGCAGGGCCG | |
| | CCTGGAGCTGTGGGTGGACATGTTCCCCATGGACATGCCAGCCCC | |
| | TGGGACGCCTCTGGACATCTCACCTCGGAAGCCCAAGAAGTACGA | |
| | GCTGCGGGTCATCATCTGGAACACAGATGAGGTGGTCTTGGAGGA | |
| | CGACGACTTCTTCACAGGGGAGAAGTCCAGTGACATCTTCGTGAG | |
| | GGGGTGGCTGAAGGGCCAGCAGGAGGACAAGCAGGACACAGACGT | |
| | CCACTACCACTCCCTCACTGGCGAGGGCAACTTCAACTGGCGCTA | |
| | CCTGTTCCCCTTCGACTACCTGGCGGCGGAGGAGAAGATCGTCAT | |
| | CTCCAAGAAGGAGTCCATGTTCTCCTGGGACGAGACCGAGTACAA | |
| | GATCCCCGCGCGGCTCACCCTGCAGATCTGGGATGCGGACCACTT | |
| | CTCCGCTGACGACTTCCTGGGGGCCATCGAGCTGGACCTGAACCG | |
| | GTTCCCGCGGGGCGCAAAGACAGCCAAGCAGTGCACCATGGAGAT | |
| | GGCCACCGGGGAGGTGGACGTGCCCCTCGTGTCCATCTTCAAGCA | |
| | AAAGCGCGTCAAAGGCTGGTGGCCCCTCCTGGCCCGCAATGAGAA | |
| | CGATGAGTTTGAGCTCACGGGCAAGGTGGAGGCTGAGCTGCATTT | |
| | ACTGACAGCAGAGGAGGCAGAGAAGAACCCAGTGGGCCTGGCCCG | |
| | CAATGAACCTGACCCCCTAGAGAAACCCAACCGGCCCGACACGGC | |
| | CTTCGTCTGGTTCCTCAACCCTCTCAAGTCCATCAAGTACCTCAT | |
| | CTGCACCCGGTACAAGTGGCTCATCATCAAGATCGTGCTGGCGCT | |
| | GTTGGGGCTGCTCATGTTGGGGCTCTTCCTCTACAGCCTCCCTGG | |
| | CTACATGGTCAAAAAGCTCCTTGGGGCATGAACTAGTGCTGATCA | |
| | GCCTCGACTGTGCCTTCTAGTTGCCAGCCATCTGTTGTTTGCCCC | |
| | TCCCCCGTGCCTTCCTTGACCCTGGAAGGTGCCACTCCCACTGTC | |
| | CTTTCCTAATAAAATGAGGAAATTGCATCGCATTGTCTGAGTAGG | |
| | TGTCATTCTATTCTGGGGGGTGGGGTGGGGCAGGACAGCAAGGGG | |
| | GAGGATTGGGAAGACAATAGCAGGCATGCTGGGGATGCGGTGGGC | |
| | TCTATGGAAGCTTGAATTCAGCTGACGTGCCTCGGACCGCCTAGG | |
| | AGGAACCCCTAGTGATGGAGTTGGCCACTCCCTCTCTGCGCGCTC | |
| | GCTCGCTCACTGAGGCCGGGCGACCAAAGGTCGCCCGACGCCCGG | |
| | GCTTTGCCCGGGCGGCCTCAGTGAGCGAGCGAGCGCGCAGAGAGG | |
| | GAGTGGCCAA | |
| 5′ITR | TTGGCCACTCCCTCTCTGCGCGCTCGCTCGCTCACTGAGGCCGCC | 97 |
| | CGGGCAAAGCCCGGGCGTCGGGCGACCTTTGGTCGCCCGGCCTCA | |
| | GTGAGCGAGCGAGCGCGCAGAGAGGGAGTGGCCAACTCCATCACT | |
| | AGGGGTTCCT | |
| AK | GGGATTTTGCCGATTTCGGCCTATTGGTTAAAAAATGAGCTGATT | 103 |
| | TAACAAAAATTTAACGCGAATTTTAACAAAAT | |
| SA intron | GATAGGCACCTATTGGTCTTACTGACATCCACTTTGCCTTTCTCT | 106 |
| | CCACAG | |
| 3′OTOF | CCCCAGCACAGCATTCCCGACATCTTCATCTGGATGATGAGCAAC | 107 |
| | AACAAGCGTGTCGCCTATGCCCGTGTGCCCTCCAAGGACCTGCTC | |
| | TTCTCCATCGTGGAGGAGGAGACTGGCAAGGACTGCGCCAAGGTC | |
| | AAGACGCTCTTCCTTAAGCTGCCAGGGAAGCGGGGCTTCGGCTCG | |
| | GCAGGCTGGACAGTGCAGGCCAAGGTGGAGCTGTACCTGTGGCTG | |
| | GGCCTCAGCAAACAGCGCAAGGAGTTCCTGTGCGGCCTGCCCTGT | |
| | GGCTTCCAGGAGGTCAAGGCAGCCCAGGGCCTGGGCCTGCATGCC | |
| | TTCCCACCCGTCAGCCTGGTCTACACCAAGAAGCAGGCGTTCCAG | |
| | CTCCGAGCGCACATGTACCAGGCCCGCAGCCTCTTTGCCGCCGAC | |
| | AGCAGCGGACTCTCAGACCCCTTTGCCCGCGTCTTCTTCATCAAT | |
| | CAGAGTCAGTGCACAGAGGTGCTGAATGAGACCCTGTGTCCCACC | |
| | TGGGACCAGATGCTGGTGTTCGACAACCTGGAGCTCTATGGTGAA | |
| | GCTCATGAGCTGAGGGACGATCCGCCCATCATTGTCATTGAAATC | |
| | TATGACCAGGATTCCATGGGCAAAGCTGACTTCATGGGCCGGACC | |
| | TTCGCCAAACCCCTGGTGAAGATGGCAGACGAGGCGTACTGCCCA | |
| | CCCCGCTTCCCACCTCAGCTCGAGTACTACCAGATCTACCGTGGC | |
| | AACGCCACAGCTGGAGACCTGCTGGCGGCCTTCGAGCTGCTGCAG | |
| | ATTGGACCAGCAGGGAAGGCTGACCTGCCCCCCATCAATGGCCCG | |
| | GTGGACGTGGACCGAGGTCCCATCATGCCCGTGCCCATGGGCATC | |
| | CGGCCCGTGCTCAGCAAGTACCGAGTGGAGGTGCTGTTCTGGGGC | |
| | CTACGGGACCTAAAGCGGGTGAACCTGGCCCAGGTGGACCGGCCA | |
| | CGGGTGGACATCGAGTGTGCAGGGAAGGGGGTGCAGTCGTCCCTG | |
| | ATCCACAATTATAAGAAGAACCCCAACTTCAACACCCTCGTCAAG | |
| | TGGTTTGAAGTGGACCTCCCAGAGAACGAGCTGCTGCACCCGCCC | |
| | TTGAACATCCGTGTGGTGGACTGCCGGGCCTTCGGTCGCTACACA | |
| | CTGGTGGGCTCCCATGCCGTCAGCTCCCTGCGACGCTTCATCTAC | |
| | CGGCCCCCAGACCGCTCGGCCCCCAGCTGGAACACCACGGTCAGG | |
| | CTTCTCCGGCGCTGCCGTGTGCTGTGCAATGGGGGCTCCTCCTCT | |
| | CACTCCACAGGGGAGGTTGTGGTGACTATGGAGCCAGAGGTACCC | |
| | ATCAAGAAACTGGAGACCATGGTGAAGCTGGACGCGACTTCTGAA | |
| | GCTGTTGTCAAGGTGGATGTGGCTGAGGAGGAGAAGGAGAAGAAG | |
| | AAGAAGAAGAAGGGCACTGCGGAGGAGCCAGAGGAGGAGGAGCCA | |
| | GACGAGAGCATGCTGGACTGGTGGTCCAAGTACTTTGCCTCCATT | |
| | GACACCATGAAGGAGCAACTTCGACAACAAGAGCCCTCTGGAATT | |
| | GACTTGGAGGAGAAGGAGGAAGTGGACAATACCGAGGGCCTGAAG | |
| | GGGTCAATGAAGGGCAAGGAGAAGGCAAGGGCTGCCAAAGAGGAG | |
| | AAGAAGAAGAAAACTCAGAGCTCTGGCTCTGGCCAGGGGTCCGAG | |
| | GCCCCCGAGAAGAAGAAACCCAAGATTGATGAGCTTAAGGTATAC | |
| | CCCAAAGAGCTGGAGTCCGAGTTTGATAACTTTGAGGACTGGCTG | |
| | CACACTTTCAACTTGCTTCGGGGCAAGACCGGGGATGATGAGGAT | |
| | GGCTCCACCGAGGAGGAGCGCATTGTGGGACGCTTCAAGGGCTCC | |
| | CTCTGCGTGTACAAAGTGCCACTCCCAGAGGACGTGTCCCGGGAA | |
| | GCCGGCTACGACTCCACCTACGGCATGTTCCAGGGCATCCCGAGC | |
| | AATGACCCCATCAATGTGCTGGTCCGAGTCTATGTGGTCCGGGCC | |
| | ACGGACCTGCACCCTGCTGACATCAACGGCAAAGCTGACCCCTAC | |
| | ATCGCCATCCGGCTAGGCAAGACTGACATCCGCGACAAGGAGAAC | |
| | TACATCTCCAAGCAGCTCAACCCTGTCTTTGGGAAGTCCTTTGAC | |
| | ATCGAGGCCTCCTTCCCCATGGAATCCATGCTGACGGTGGCTGTG | |
| | TATGACTGGGACCTGGTGGGCACTGATGACCTCATTGGGGAAACC | |
| | AAGATCGACCTGGAGAACCGCTTCTACAGCAAGCACCGCGCCACC | |
| | TGCGGCATCGCCCAGACCTACTCCACACATGGCTACAATATCTGG | |
| | CGGGACCCCATGAAGCCCAGCCAGATCCTGACCCGCCTCTGCAAA | |
| | GACGGCAAAGTGGACGGCCCCCACTTTGGGCCCCCTGGGAGAGTG | |
| | AAGGTGGCCAACCGCGTCTTCACTGGGCCCTCTGAGATTGAGGAC | |
| | GAGAACGGTCAGAGGAAGCCCACAGACGAGCATGTGGCGCTGTTG | |
| | GCCCTGAGGCACTGGGAGGACATCCCCCGCGCAGGCTGCCGCCTG | |
| | GTGCCAGAGCATGTGGAGACGAGGCCGCTGCTCAACCCCGACAAG | |
| | CCGGGCATCGAGCAGGGCCGCCTGGAGCTGTGGGTGGACATGTTC | |
| | CCCATGGACATGCCAGCCCCTGGGACGCCTCTGGACATCTCACCT | |
| | CGGAAGCCCAAGAAGTACGAGCTGCGGGTCATCATCTGGAACACA | |
| | GATGAGGTGGTCTTGGAGGACGACGACTTCTTCACAGGGGAGAAG | |
| | TCCAGTGACATCTTCGTGAGGGGGTGGCTGAAGGGCCAGCAGGAG | |
| | GACAAGCAGGACACAGACGTCCACTACCACTCCCTCACTGGCGAG | |
| | GGCAACTTCAACTGGCGCTACCTGTTCCCCTTCGACTACCTGGCG | |
| | GCGGAGGAGAAGATCGTCATCTCCAAGAAGGAGTCCATGTTCTCC | |
| | TGGGACGAGACCGAGTACAAGATCCCCGCGCGGCTCACCCTGCAG | |
| | ATCTGGGATGCGGACCACTTCTCCGCTGACGACTTCCTGGGGGCC | |
| | ATCGAGCTGGACCTGAACCGGTTCCCGCGGGGCGCAAAGACAGCC | |
| | AAGCAGTGCACCATGGAGATGGCCACCGGGGAGGTGGACGTGCCC | |
| | CTCGTGTCCATCTTCAAGCAAAAGCGCGTCAAAGGCTGGTGGCCC | |
| | CTCCTGGCCCGCAATGAGAACGATGAGTTTGAGCTCACGGGCAAG | |
| | GTGGAGGCTGAGCTGCATTTACTGACAGCAGAGGAGGCAGAGAAG | |
| | AACCCAGTGGGCCTGGCCCGCAATGAACCTGACCCCCTAGAGAAA | |
| | CCCAACCGGCCCGACACGGCCTTCGTCTGGTTCCTCAACCCTCTC | |
| | AAGTCCATCAAGTACCTCATCTGCACCCGGTACAAGTGGCTCATC | |
| | ATCAAGATCGTGCTGGCGCTGTTGGGGCTGCTCATGTTGGGGCTC | |
| | TTCCTCTACAGCCTCCCTGGCTACATGGTCAAAAAGCTCCTTGGG | |
| | GCA | |
| bGHpA | CTGTGCCTTCTAGTTGCCAGCCATCTGTTGTTTGCCCCTCCCCCG | 108 |
| | TGCCTTCCTTGACCCTGGAAGGTGCCACTCCCACTGTCCTTTCCT | |
| | AATAAAATGAGGAAATTGCATCGCATTGTCTGAGTAGGTGTCATT | |
| | CTATTCTGGGGGGTGGGGTGGGGCAGGACAGCAAGGGGGAGGATT | |
| | GGGAAGACAATAGCAGGCATGCTGGGGATGCGGTGGGCTCTATGG | |
| 3′ITR | AGGAACCCCTAGTGATGGAGTTGGCCACTCCCTCTCTGCGCGCTC | 104 |
| | GCTCGCTCACTGAGGCCGGGCGACCAAAGGTCGCCCGACGCCCGG | |
| | GCTTTGCCCGGGCGGCCTCAGTGAGCGAGCGAGCGCGCAGAGAGG | |
| | GAGTGGCCAA | |

TABLE 3
Anc80L65

| | | SEQ |
|---|---|---|
| | | ID |
| Name | SEQUENCE | NO |
| Anc80L65 | MAADGYLPDWLEDNLSEGIREWWDLKPGAPKPKANQQKODDGRGL | 109 |
| | VLPGYKYLGPFNGLDKGEPVNAADAAALEHDKAYDQQLKAGDNPY | |
| | LRYNHADAEFQERLQEDTSFGGNLGRAVFQAKKRVLEPLGLVEEG | |
| | AKTAPGKKRPVEQSPQEPDSSSGIGKKGQQPARKRLNFGQTGDSE | |
| | SVPDPQPLGEPPAAPSGVGSNTMAAGGGAPMADNNEGADGVGNAS | |
| | GNWHCDSTWLGDRVITTSTRTWALPTYNNHLYKQISSQSGGSTND | |
| | NTYFGYSTPWGYFDFNRFHCHFSPRDWORLINNNWGFRPKKLNFK | |
| | LFNIQVKEVTTNDGTTTIANNLTSTVQVFTDSEYQLPYVLGSAHQ | |
| | GCLPPFPADVFMIPQYGYLTLNNGSQAVGRSSFYCLEYFPSQMLR | |
| | TGNNFQFSYTFEDVPFHSSYAHSQSLDRLMNPLIDQYLYYLSRTQ | |
| | TTSGTAGNRTLQFSQAGPSSMANQAKNWLPGPCYRQQRVSKTTNQ | |
| | NNNSNFAWTGATKYHLNGRDSLVNPGPAMATHKDDEDKFFPMSGV | |
| | LIFGKQGAGNSNVDLDNVMITNEEEIKTTNPVATEEYGTVATNLQ | |
| | SANTAPATGTVNSQGALPGMVWQDRDVYLQGPIWAKIPHTDGHFH | |
| | PSPLMGGFGLKHPPPQILIKNTPVPANPPTTFSPAKFASFITQYS | |
| | TGQVSVEIEWELQKENSKRWNPEIQYTSNYNKSTNVDFAVDTNGV | |
| | YSEPRPIGTRYLTRNL* | |

### Example 5: In Vivo Expression of Full-Length Human Otoferlin Using Two Vectors

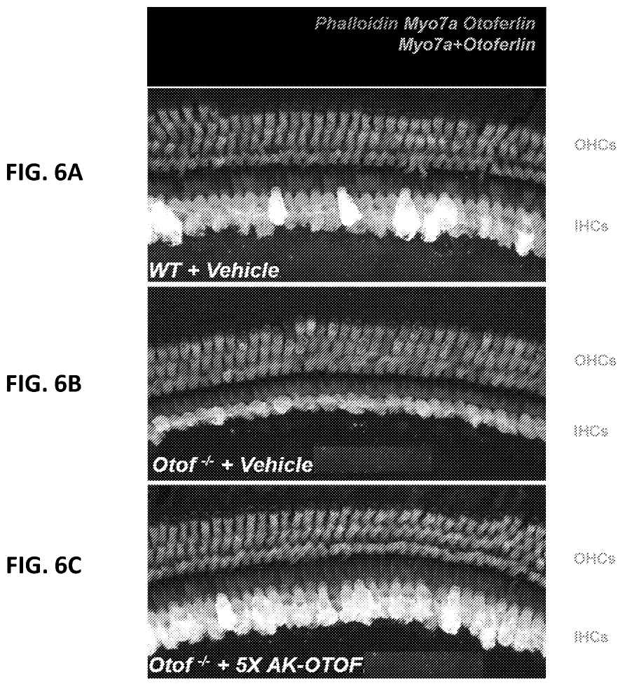

Wildtype or Otof^{-/-} mice (p23±2 days) were administered either vehicle or the AAVAnc80-hOTOF dual hybrid vectors described in Example 4 via intra-cochlear injection. Otoferlin expression in the inner hair cells was examined 1 month post-administration (FIGS. 6A-6C). Robust expression of full-length human otoferlin was observed only in the inner hair cells and not other cochlear cells in Otof^{-/-} mice administered AAVAnc80-hOTOF dual hybrid vectors (FIG. 6C).

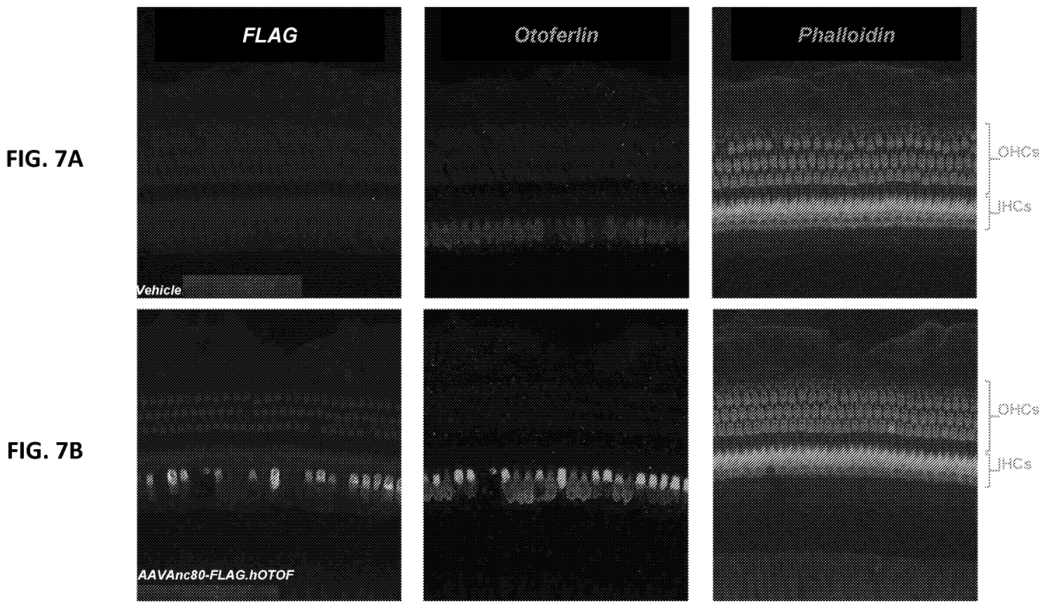

Non-human primates (NHP) were administered either vehicle or the flag-tagged AAVAnc80-hOTOF dual hybrid vectors described in Example 4 via intra-cochlear injection. Otoferlin expression in the inner hair cells was examined 1 month post-administration (FIGS. 7A-7B). Otoferlin-Flag was detected only in the inner hair cells and not in other cochlear neural regions or supporting cell regions (FIG. 7B).

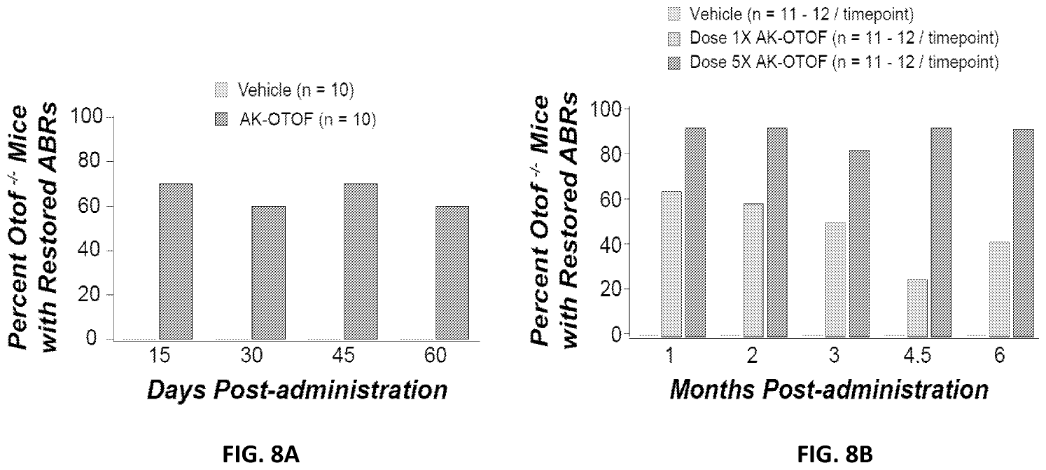

Next, the auditory function of Otof^{-/-} mice administered either vehicle or the AAVAnc80-hOTOF dual hybrid vectors described in Example 4 was assessed. The auditory brainstem response (ABR) was assessed 15, 30, 45, and 60 days (FIG. 8A) or 1, 2, 3, 4.5, or 6 months (FIG. 8B) post-administration. Otof/mice administered vehicle had no measurable auditory brainstem response (FIGS. 8A-8B). Approximately 70% of Otof^{-/-} mice administered the AAVAnc80-hOTOF dual hybrid vectors showed restoration of the auditory brainstem response by day 15 (FIG. 8A). At least 80% of Otof−/− administered AAVAnc80-Otoferlin dual hybrid vectors at dose 5× had restored auditory brainstem response through at least 6 months (FIG. 8B). The extent of auditory function restoration was dependent on the dose administered.

### Example 6: In Vivo Biodistribution of Full-Length Human Otoferlin Using Two Vectors

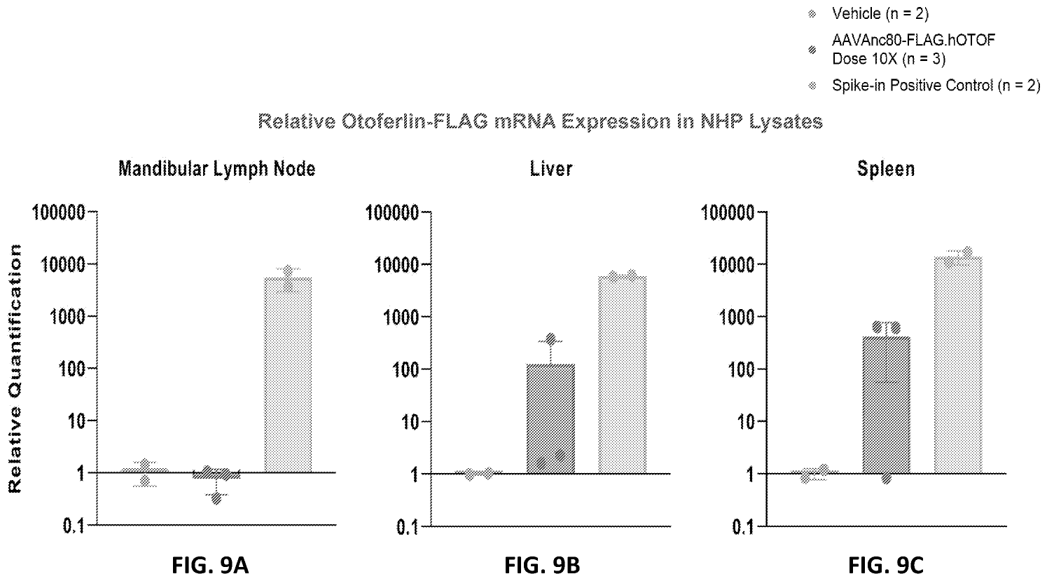

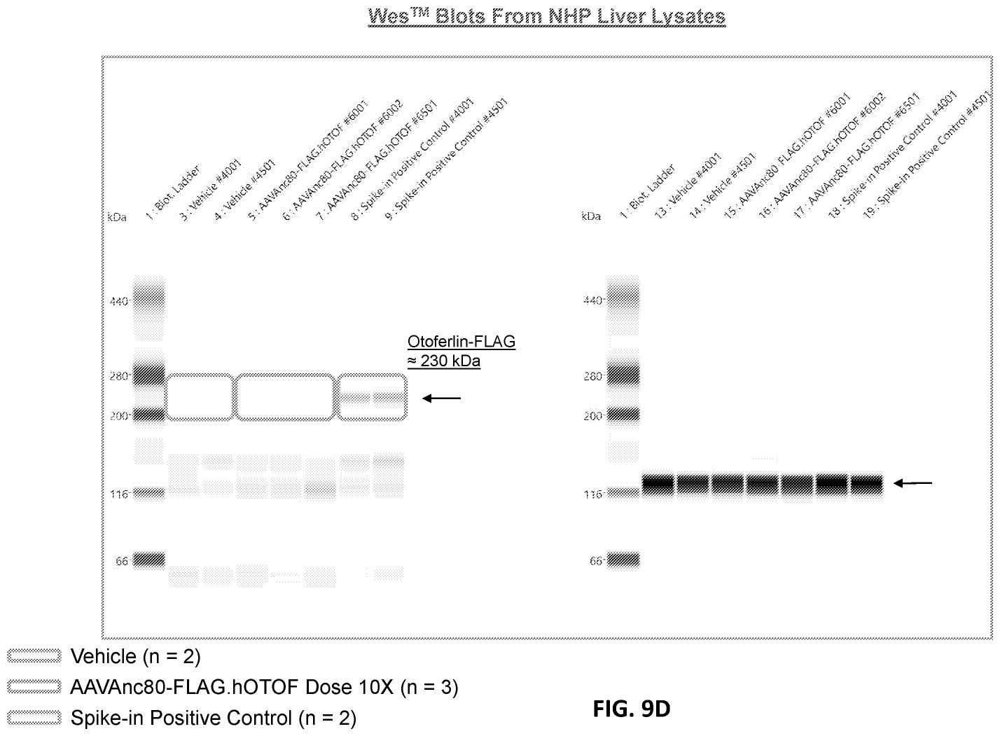

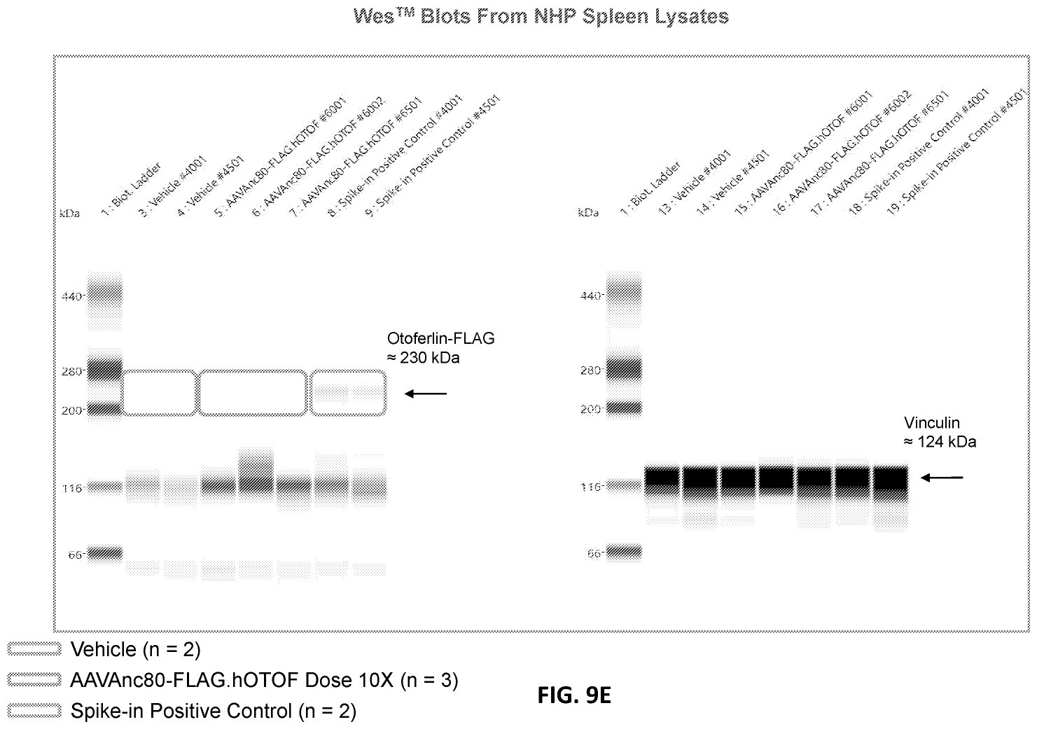

Non-human primates received bilateral intracochlear administration of either vehicle, the flag-tagged AAVAnc80-hOTOF dual hybrid vectors described in Example 4, or spike-in positive control (OTOF transduced HEK293FT cell lysate added to NHP tissue lysate) and biodistribution of Otoferlin in the mandibular lymph node, liver, and spleen were assessed 1 month post-administration (FIGS. 9A-9C). Only the liver and spleen were positive for human Otoferlin-Flag mRNA expression by RT-qPCR and only in a proportion of animals (FIGS. 9A-9C). No human Otoferlin-Flag protein was detected in the liver or spleen by Western blotting (FIGS. 9D-9E).

### Example 7: In Vivo Cochlear Hair Cell Survival and Function after Administration of Full-Length Human Otoferlin Using Two Vectors

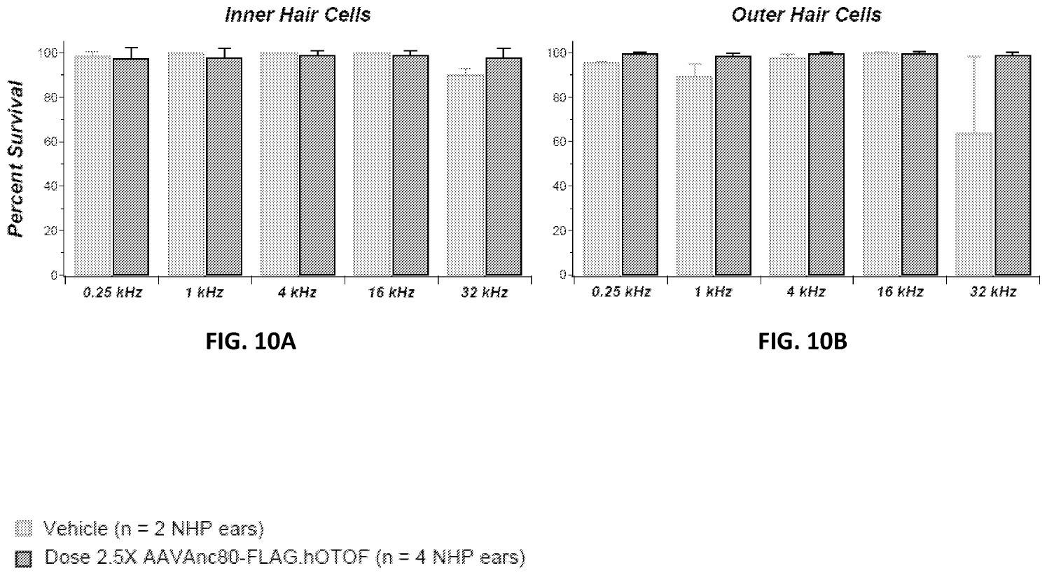

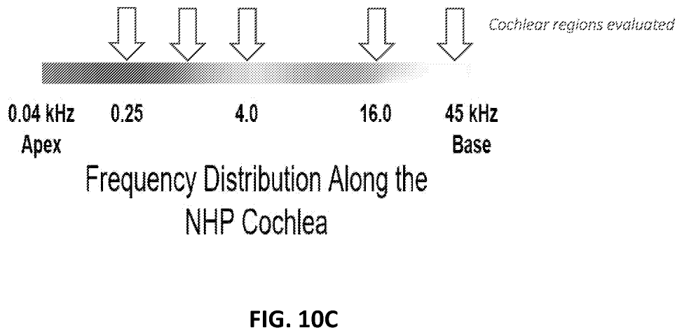

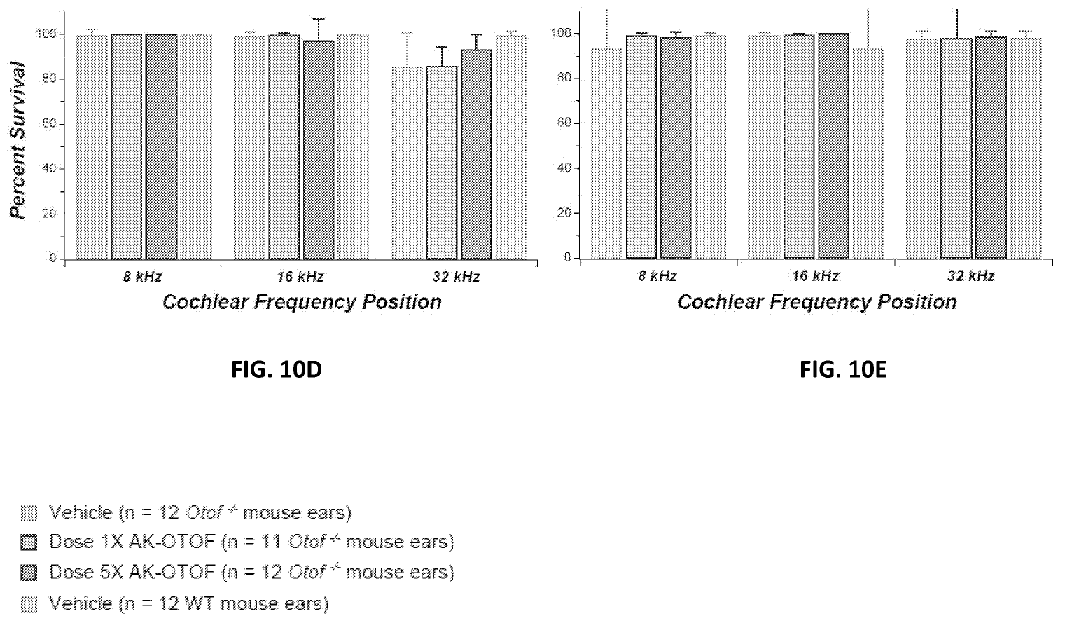

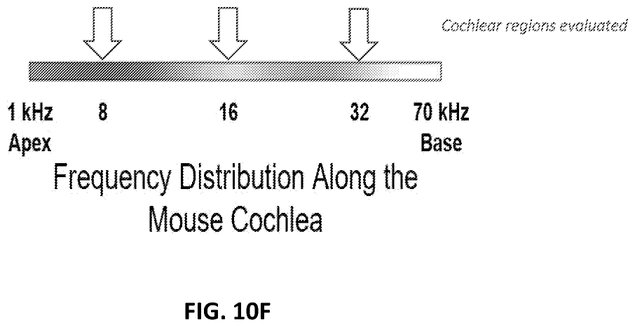

Cochlear hair cell survival was quantified to assess local tolerability of intracochlear administration of either vehicle or the flag-tagged AAVAnc80-hOTOF dual hybrid vectors described in Example 4 to non-human primates (NHP) (FIGS. 10A-10C) or Otof mice (FIGS. 10D-10F). AAVAnc80-Otof was systemically and locally well tolerated and no adverse effects were observed in clinical pathology, otic pathlogy, systemtic histopathology, and/or auditory functions (FIGS. 10A-10F).

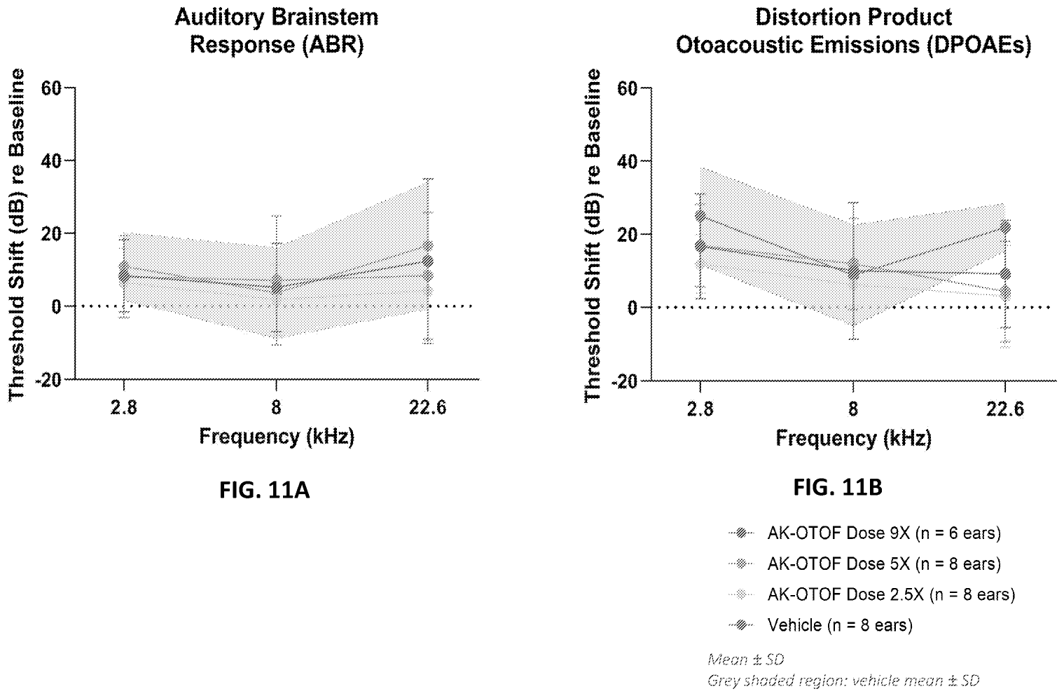

Auditory and cochlear function were evaluated pre and 6 months post-intracochlear administration of either vehicle or the flag-tagged AAVAnc80-hOTOF dual hybrid vectors described in Example 4 to non-human primates (NHP) (FIGS. 11A-11B). The auditory brainstem response (ABR) was used to measure auditory function and distortion product optoacoustic emissions (DPOAEs) was used to measure cochlear function. No impact of Otoferlin expression or dose was observed within-ear shifts (pre-vs. post-administration) in ABR or DPOAE thresholds.

### Other Aspects

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described aspects, it is not intended that it should be limited to any such particulars or aspects or any particular aspect, but it is to be construed with references to the appended claims so as to provide the broadest possible interpretation of such claims in view of the prior art and, therefore, to effectively encompass the intended scope of the disclosure.

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A composition comprising:
a) a first recombinant adeno-associated viral (rAAV) vector genome (vg) comprising a first expression cassette comprising a promoter operably linked to nucleic acid sequence comprising a 5′ portion of an otoferlin gene, wherein the expression cassette is flanked by inverted terminal repeats (ITRs); and
b) a second rAAV vector genome comprising a second expression cassette comprising a nucleic acid sequence comprising a 3′ portion of an otoferlin gene, wherein the expression cassette is flanked by ITRs;
wherein the composition comprises about 4.1E10-4.1E12 total vg or about 8.1E10-8.1E12 total vg.

2.-199. (canceled)

200. The composition of claim 1, wherein the composition comprises about 4.5E11-4.5E13 total vg/mL or about 9E11-9E13 total vg/mL.

201. The composition of claim 1, wherein the composition comprises about 4.5E12 total vg/mL or about 9E12 total vg/mL.

202. The composition of claim 1, wherein the composition comprises the first rAAV vector genome and the second rAAV vector genome in about a 1:1 ratio.

203. The composition of claim 1, wherein the nucleic acid sequence comprising the 5′ portion of the otoferlin gene encodes the portion of an otoferlin protein that is encoded by SEQ ID NO: 101; and wherein the nucleic acid sequence comprising the 3′ portion of the otoferlin gene encodes the portion of an otoferlin protein that is encoded by SEQ ID NO: 107.

204. The composition of claim 1, wherein the promoter is a CAG promoter.

205. A composition comprising:
a) a first rAAV vector genome comprising a first expression cassette comprising a promoter, a first coding sequence that encodes an N-terminal portion of an otoferlin protein positioned 3′ of the promoter, and a splicing donor signal sequence positioned at the 3′ end of the first coding sequence; and
b) a second rAAV vector genome comprising a second expression cassette comprising a splicing acceptor signal sequence, a second coding sequence that encodes a C-terminal portion of an otoferlin protein positioned at the 3′ end of the splicing acceptor signal sequence, and a polyadenylation sequence at 3′ end of the second coding sequence;
wherein the composition is formulated for intra-cochlear administration and comprises monopotassium phosphate, sodium phosphate dibasic, potassium chloride, sodium chloride, and Poloxamer 188.

206. The composition of claim 205, wherein the formulation comprises:
a) about 1.35-1.65 mM monopotassium phosphate;
b) about 7.29-8.91 mM sodium phosphate dibasic;
c) about 2.43-2.97 mM potassium chloride;
d) about 154.8-189.2 mM sodium chloride; and
e) about 0.0001%-0.01% Poloxamer 188.

207. The composition of claim 206, wherein the formulation comprises:
a) about 1.5 mM monopotassium phosphate;
b) about 8.1 mM sodium phosphate dibasic;
c) about 2.7 mM potassium chloride;
d) about 172 mM sodium chloride; and
e) about 0.001% Poloxamer 188.

208. The composition of claim 205, wherein the composition comprises about 4.5E11-4.5E13 total vg/mL or about 9E11-9E13 total vg/mL.

209. The composition of claim 208, wherein the composition comprises about 4.5E12 total vg/mL or about 9E12 total vg/mL.

210. The composition of claim 205, wherein the first rAAV vector and second rAAV vector each comprises a recombinogenic sequence.

211. The composition of claim 205, wherein the polyadenylation sequence is selected from a bovine growth hormone, human growth hormone, mouse-β-globin, mouse-α-globin, polyomavirus, SV40, or synthetic polyadenylation sequence.

212. The composition of claim 205, wherein each of the first and second expression cassettes is flanked by inverted terminal repeats (ITRs), and wherein the ITRs are selected from any one of AAV1, AAV2, AAV3, AAV4, AAV5, AAV6, AAV7, AAV8, AAV9, AAVrh8, AAVrh10, AAVrh39, AAVrh43, o Anc80 ITRs.

213. The composition of claim 212, wherein the first and second rAAV vector genomes are each encapsulated by an AAV capsid.

214. The composition of claim 213, wherein the AAV capsid is a serotype selected from any one of AAV1, AAV2, AAV3, AAV4, AAV5, AAV6, AAV7, AAV8, AAV9, AAVrh8, AAVrh10, AAVrh39, AAVrh43, or Anc80.

215. The composition of claim 214, wherein the AAV capsid comprises the polypeptide sequence of SEQ ID NO: 109.

216. A method of expressing a recombinant full-length otoferlin protein in a mammalian cell, the method comprising administering the composition of claim 205 to the mammalian cell, wherein the first and second rAAV vector genomes are capable of constituting a messenger RNA encoding a full length human otoferlin protein in the mammalian cell.

217. The method of claim 216, wherein the mammalian cell is an inner ear hair cell.

218. A method of treating hearing loss in a subject having a defective otoferlin gene, the method comprising administering the composition claim 1 to the cochlea of the subject, wherein the first and second rAAV vector genomes are capable of constituting a messenger RNA encoding a full length human otoferlin protein in the subject.

219. The method of claim 218, wherein the subject is a human.

220. The method of claim 218, wherein the composition is administered in a single dose.

221. The method of claim 220, wherein the single dose comprises about 0.01-0.2 mL.

222. The method of claim 220, wherein the single dose comprises about 0.09 mL.

223. The method of claim 218, wherein about 4.1E10-4.1E12 total vg, or about 8.1E10-8.1E12 total vg, is administered to the cochlea of the subject.

224. The method of claim 218, wherein about 4.1E11 total vg or about 8.1E11 total vg is administered per cochlea.

225. A kit comprising the composition of claim 1 pre-loaded into a device or a vial.

226. The kit of claim 225, wherein the device comprises a microcatheter.

227. The kit of claim 226, wherein the microcatheter is shaped such that it can enter the middle ear cavity and contact the round window membrane (RWM).

228. The kit of claim 225, further comprising a second vial comprising a diluent.
